(19) United States
(12) Patent Application Publication
(10) Pub. No.: US 2013/0029920 A1
(43) Pub. Date: Jan. 31, 2013

(54) Metal Nanoparticles

(71) Applicants: Margaret A. Brimble, Auckland (NZ); Stefanie Papst, Auckland (NZ); Raoul Peltier, Auckland (NZ); Richard D. Tilley, Wellington (NZ); David E. Williams, Auckland (NZ)

(72) Inventors: Margaret A. Brimble, Auckland (NZ); Stefanie Papst, Auckland (NZ); Raoul Peltier, Auckland (NZ); Richard D. Tilley, Wellington (NZ); David E. Williams, Auckland (NZ)

(21) Appl. No.: 13/561,527

(22) Filed: Jul. 30, 2012

(30) Foreign Application Priority Data
Jul. 29, 2011 (NZ) 594348
Mar. 21, 2012 (NZ) 598940

(51) Int. Cl. A61K 38/10; B01J 31/26; A61K 38/08; B82Y 40/00; B82Y 30/00

(52) U.S. Cl. 514/21.4; 502/167; 514/21.5; 514/21.6; 514/21.7; 977/773; 977/892

(57) ABSTRACT

A metal nanoparticle-phosphopeptide complex comprising a metal nanoparticle and a phosphopeptide is provided. The phosphopeptide comprises two or more contiguous peptide motifs and two or more phosphorus-containing groups capable of interacting with the surface of the metal nanoparticle. The amino acids at the equivalent position in each peptide motif have similar structural and/or electronic properties. Each phosphorus-containing group is bound to an amino acid in the two or more contiguous peptide motifs. Methods for preparing the metal nanoparticle-phosphopeptide complex are also provided.

### TECHNICAL FIELD

The present invention generally relates to metal nanoparticles. In particular, the present invention relates to metal nanoparticle-phosphopeptide complexes, methods for preparing metal nanoparticles and metal nanoparticle-phosphopeptide complexes, and to metal nanoparticles and metal nanoparticle-phosphopeptide complexes prepared according to those methods. The present invention also relates to phosphopeptides and to compositions comprising metal nanoparticles and those phosphopeptides, and to compositions comprising metal nanoparticle-phosphopeptide complexes, and kits. The present invention also relates to uses of the metal nanoparticles and metal nanoparticle-phosphopeptide complexes in the manufacture of medicaments, in methods of treatment and imaging, and as catalysts.

### BACKGROUND OF THE INVENTION

Metallic nanoparticles exhibit unusual optical, thermal, chemical and physical properties, due to the large proportion of high-energy surface atoms compared to bulk solid and to the nanometer-scale mean free path of electrons in the metal (˜10-100 nm for many metals at room temperature.

Some metal nanoparticles have significant potential as catalysts that, due to the ability to lower the activation energy of certain reactions, facilitate the synthesis of important chemicals. Many transition metals in their bulk state already possess catalytic properties. Nanoparticles of such metals can have significantly greater catalytic activities, due to the large specific surface areas of the nanoparticles, which may open further fields of application.

Metal nanoparticles with increased the catalytic activity, relative to bulk metal, allow the amount of the metal used to be reduced while preserving the same level of catalyst performance. This can provide significant cost benefits.

Metal nanoparticles may also have useful magnetic properties. Magnetic nanoparticles are currently used as magnetic media storage, but are also of great interest for their potential applications in medicine. Potential medicinal applications include cancer treatment by hyperthermia, contrast enhancement in medical imaging, new drug delivery methods, etc.

Iron nanoparticles are of particular interest. Iron nanoparticles exhibit strong ferromagnetic or ferrimagnetic behaviour, and super-paramagnetic properties when the particle size is less than about 10 nm. As a result, iron is the most widely used metal for the preparation of magnetic nanoparticles and their applications.

Many methods are available for the synthesis of metal nanoparticles: thermal or sonochemical decomposition, hydrothermal synthesis, vapor phase synthesis, laser pyrolysis, etc. However, these methods typically require the use of complex equipment, high temperatures, high pressures, and/or harsh organic solvents.

Wet chemical techniques involving, for example, the reduction of metal salts have proven to be particularly efficient and can be performed in water without any special equipment (see, for example, K. J. Carroll et al., J. Appl. Phys., 2010, 107 and R. Lu et al., Cryst. Growth Des., 2007, 7, 459-464). Additives that template the growth of the nanoparticles and prevent their aggregation are commonly used in these techniques.

The most commonly used additives are surfactants. The surfactants form reverse-micelles or microemulsions inside which the metal nanoparticles grow. The size of the nanoparticles is limited by the size of the reverse-micelles (see, for example, A. Martino et al., Applied Catalysis a-General, 1997, 161, 235-248).

Additives that adsorb on the growing face of nanoparticles and modify its normal growth have been reported. For example, Guo et al. have used trioctylphosphine oxide (TOPO) capping reagents to prepare highly monodisperse iron nanoparticles (L. Guo et al., Phys. Chem. Chem. Phys., 2001, 3, 1661-1665).

The additives remain attached to the surface of the nanoparticles after synthesis and may adversely affect the biocompatibility and toxicity of the nanoparticles for medical applications.

Examples of more “green” methods for preparing metal nanoparticles using plant extracts in aqueous solution have recently been reported (E. C. Njagi et al., Langmuir, 2011, 27, 264-271 and M. N. Nadagouda et al., Green Chemistry, 2010, 12, 114-122). However, these methods generally led to particles having poor polydispersity.

There is a need for new methods for preparing metal nanoparticles.

It is an object of the present invention to go some way to meeting this need; and/or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides a metal nanoparticle-phosphopeptide complex comprising:
a metal nanoparticle; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs.

The metal nanoparticle comprises at least one metal. In one embodiment, the metal nanoparticle comprises a single metal. In another embodiment, the metal nanoparticle comprises a mixture of two or more metals.

In one embodiment, the metal is selected from the metals in groups 3 to 12 of the periodic table. In another embodiment, the metal is selected from the metals in groups 3 to 12, 4 to 12, 5 to 12, 6 to 12, 7 to 12, or 8 to 12 of the periodic table. In another embodiment, the metal is selected from the metals in groups 3 to 11, 4 to 11, 5 to 11, 6 to 11, 7 to 11, or 8 to 11 of the periodic table. In one exemplary embodiment, the metal is selected from the metals in groups 8 to 11 of the periodic table. In one embodiment, the metal is selected from the metals in periods 4 to 6 of the periodic table. In one exemplary embodiment, the metal is selected from the metals in periods 4 to 6 and groups 8 to 11 of the periodic table.

In one specifically contemplated embodiment, the metal is selected from the group consisting of iron, cobalt, nickel, copper, ruthenium, rhodium, palladium, silver, iridium, platinum, and gold. In one specifically contemplated embodiment, the metal is selected from the group consisting of iron, cobalt, ruthenium, rhodium, palladium, silver, platinum, and gold. In another specifically contemplated embodiment, the metal is selected from the group consisting of iron, cobalt, ruthenium, rhodium, palladium, silver, and gold. In another specifically contemplated embodiment, the metal is selected from the group consisting of iron, ruthenium, rhodium, palladium, silver, and gold. In another specifically contemplated embodiment, the metal is selected from the group consisting of iron, ruthenium, palladium, and gold. In another specifically contemplated embodiment, the metal is iron.

In one embodiment, the metal nanoparticle is an iron nanoparticle. In one embodiment, the iron nanoparticle is an iron oxide nanoparticle. In one embodiment, the size of the iron oxide nanoparticle is from about 5 nm to about 8 nm.

In another embodiment, the iron nanoparticle is an iron-iron oxide core-shell nanoparticle. In one embodiment, the size of the iron-iron oxide core-shell nanoparticle is from about 8 nm to about 25 nm. In another embodiment, the size of the iron-iron oxide core-shell nanoparticle is from about 15 nm to about 25 nm.

In one embodiment, the metal nanoparticle is a cobalt nanoparticle.

In one embodiment, the metal nanoparticle is a nickel nanoparticle.

In one embodiment, the metal nanoparticle is a copper nanoparticle.

In one embodiment, the metal nanoparticle is a ruthenium nanoparticle. In one embodiment, the size of the ruthenium nanoparticle is from about 20 nm to 100 nm.

In one embodiment, the metal nanoparticle is a rhodium nanoparticle.

In one embodiment, the metal nanoparticle is a palladium nanoparticle. In one embodiment, the size of the palladium nanoparticle is from about 3 nm to about 7 nm. In another embodiment, the size of the palladium nanoparticle is about 5 nm.

In one embodiment, the metal nanoparticle is a silver nanoparticle.

In one embodiment, the metal nanoparticle is an iridium nanoparticle.

In one embodiment, the metal nanoparticle is a platinum nanoparticle.

In one embodiment, the metal nanoparticle is a gold nanoparticle. In one embodiment, the size of the gold nanoparticle is from about 3 nm to about 5 nm. In another embodiment, the size of the gold nanoparticle is about 4 nm.

In one embodiment, the metal nanoparticle exhibits ferromagnetic behaviour at room temperature. In another embodiment, the metal nanoparticle exhibits ferrimagnetic behaviour at room temperature. In another embodiment, the metal nanoparticle exhibits super-paramagnetic behaviour at room temperature. In one embodiment, the metal nanoparticle comprises iron, cobalt, nickel, or a mixture of any two or more thereof. In another embodiment, the metal nanoparticle comprises iron or a mixture of iron and cobalt, iron and nickel, or iron, cobalt, and nickel. In another embodiment, the metal nanoparticle comprises iron. In another embodiment, the metal nanoparticle is an iron nanoparticle.

In one embodiment, the molar ratio of metal to phosphopeptide is more than about 1:1, 2:1, 3:1, 4:1, 5:1, 6:1, 7:1, 8:1, 9:1, 10:1, 15:1, or 20:1. In one embodiment, the molar ratio is from about 1:1 to 25:1, 1:1 to 20:1, 1:1 to 15:1, 1:1 to 10:1, 1:1 to 5:1, 5:1 to 25:1, 5:1 to 20:1, 5:1 to 15:1, 5:1 to 10:1, 10:1 to 25:1, 10:1 to 20:1, 10:1 to 15:1, 15:1 to 25:1, 15:1 to 20:1, or 20:1 to 25:1.

In one embodiment, the phosphorus-containing group comprises a phosphate or phosphonate group.

In one embodiment, the phosphopeptide favours a helical structure in solution.

In one embodiment, the amino acid sequence of the two or more contiguous peptide motifs is such that the contiguous peptide motifs favour an amphipathic helical structure in solution.

In one embodiment, the amino acid sequence of the two or more contiguous peptide motifs is such that the phosphopeptide favours an amphipathic helical structure in solution.

In one embodiment, each peptide motif is 3 or more amino acids in length. In one embodiment, each peptide motif is 3 to 7 amino acids in length. In another embodiment, each peptide motif is 3, 4, or 5 amino acids in length. In another embodiment, each peptide motif is 3 amino acids in length.

In one embodiment, the two or more phosphorus-containing groups are bound to amino acids at the equivalent position in each peptide motif.

In one embodiment, each amino acid is selected from one of the following categories: polar amino acids, non polar amino acids, hydrophobic amino acids, and non hydrophobic amino acids; and the amino acids at the equivalent position in each peptide motif are selected from the same category of amino acids.

In one embodiment, each amino acid is independently an amino acid residue of the formula (II):

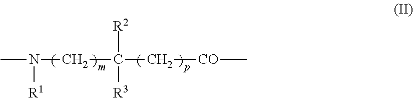

wherein:
R¹ is selected from the group consisting of hydrogen, C₁₋₆alkyl, C₂₋₆alkenyl, and C₂₋₆alkynyl, each of which is optionally substituted with one or more substituents independently selected from the group consisting of hydroxyl, C₁₋₆alkyoxy, thiol, C₁₋₆alkylthio, halo, C₁₋₆haloalkoxy, cyano, nitro, amino, and carboxyl;
R² is selected from the group consisting of hydrogen, C₁₋₆alkyl, C₂₋₆alkenyl, C₂₋₆ alkynyl, C₃₋₁₀cycloalkyl, C₁₋₆alkylC₃₋₁₀cycloalkyl, C₂₋₆alkenylC₃₋₁₀cycloalkyl, C₂₋₆ alkynylC₃₋₁₀cycloalkyl, C₃₋₁₀cycloalkenyl, C₁₋₆alkylC₃₋₁₀cycloalkenyl, C₂₋₆alkenylC₃₋₁₀cycloalkenyl, C₂₋₆alkynylC₃₋₁₀cycloalkenyl, aryl, C₁₋₆alkylaryl, C₂₋₆alkenylaryl, C₂₋₆alkynylaryl, heteroaryl, C₁₋₆alkylheteroaryl, C₂₋₆alkenylheteroaryl, C₂₋₆alkynylheteroaryl, heterocyclyl, C₁₋₆alkylheterocyclyl, C₂₋₆alkenylheterocyclyl, and C₂₋₆alkynylheterocyclyl, each of which is optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, acyl, amino, amido, acylamino, carboxyl, acyloxy, guanidino, urea, carbonate, thiourea, cyano, nitro, nitroso, azide, cyanate, thiocyanate, and isocyanate;
R³ is selected from the group consisting of hydrogen, C₁₋₆alkyl, C₂₋₆alkenyl, and C₂₋₆alkynyl, each of which is optionally substituted with one or more substituents independently selected from the group consisting of hydroxyl, C₁₋₆alkyoxy, thiol, C₁₋₆alkylthio, halo, C₁₋₆haloalkoxy, cyano, nitro, amino, and carboxyl;
or R¹ and R² together with nitrogen atom and carbon atom to which they are attached form a 5- or 6-membered heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, cyano, and nitro;
or R² and R³ together with the carbon atom to which they are attached form a 5- or 6-membered cycloalkyl, cycloalkenyl, or heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, acyl, amino, amido, acylamino, carboxyl, acyloxy, guanidino, urea, carbonate, thiourea, cyano, nitro, nitroso, azide, cyanate, thiocyanate, and isocyanate;
m is an integer from 0 to 2 and p is 0, or m is 0 and p is an integer from 0 to 2.

In one embodiment, each phosphorus-containing group is bound to an amino acid residue of the formula (II).

In one embodiment, each phosphorus-containing group is bound to R², the optionally substituted ring formed when R¹ and R² are taken together with nitrogen atom and carbon atom to which they are attached, or the optionally substituted ring formed when R² and R³ are taken together with the carbon atom to which they are attached.

In one embodiment, the phosphorus-containing group is selected from the group consisting of phosphate, C₁₋₆alkylphosphate, C₂₋₆alkenylphosphate, C₂₋₆alkynylphosphate, arylphosphate, C₁₋₆alkylarylphosphate, C₂₋₆alkenylarylphosphate, C₂₋₆alkynylarylphosphate, phosphonate, C₁₋₆alkylphosphonate, C₂₋₆alkenylphosphonate, C₂₋₆alkynylphosphonate, arylphosphonate, C₁₋₆alkylarylphosphonate, C₂₋₆alkenylarylphosphonate, C₂₋₆alkynylarylphosphonate. In one embodiment, the phosphorus-containing group is selected from the group consisting of phosphate, phosphonate, C₁₋₆alkylphosphate, and C₁₋₆alkylphosphonate.

In one embodiment, each amino acid is independently an amino acid residue of the formula (II) wherein:
R¹ is selected from the group consisting of hydrogen, C₁₋₆alkyl, C₂₋₆alkenyl, and C₂₋₆alkynyl, each of which is optionally substituted with one or more halo;
R² is selected from the group consisting of C₁₋₆alkyl, C₂₋₆alkenyl, C₂₋₆alkynyl, C₁₋₆alkylaryl, and C₁₋₆alkylheteroaryl, wherein each C₁₋₆alkyl, C₂₋₆alkenyl, and C₂₋₆alkynyl is substituted with hydroxyl, thiol, amino, amido, carboxyl, or guanidino, and optionally substituted with one or more substituents independently selected from the group consisting of hydroxyl, C₁₋₆alkyoxy, thiol, C₁₋₆alkylthio, halo, C₁₋₆haloalkyl, C₁₋₆haloalkoxy, cyano, and nitro, each C₁₋₆alkylaryl is substituted with hydroxyl, thiol, or amino, and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro, and each C₁₋₆alkylheteroaryl is optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro;
R³ is selected from the group consisting of hydrogen, C₁₋₆alkyl, C₂₋₆alkenyl, and C₂₋₆alkynyl, each of which is optionally substituted with one or more halo;
or R¹ and R² together with nitrogen atom and carbon atom to which they are attached form a 5- or 6-membered heterocyclyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, cyano, and nitro;
or R² and R³ together with the carbon atom to which they are attached form a 5- or 6-membered cycloalkyl or cycloalkenyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, or a 5- or 6-membered heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy; and
m is 0 or 1 and p is 0, or m is 0 and p is 0 or 1.

In one embodiment, each phosphorus-containing group is bound to R², the optionally substituted ring formed when R¹ and R² are taken together with nitrogen atom and carbon atom to which they are attached, or the optionally substituted ring formed when R² and R³ are taken together with the carbon atom to which they are attached.

In one embodiment, the phosphorus-containing group is selected from the group consisting of phosphate, C₁₋₆alkylphosphate, C₂₋₆alkenylphosphate, C₂₋₆alkynylphosphate, arylphosphate, C₁₋₆alkylarylphosphate, C₂₋₆alkenylarylphosphate, C₂₋₆alkynylarylphosphate, phosphonate, C₁₋₆alkylphosphonate, C₂₋₆alkenylphosphonate, C₂₋₆alkynylphosphonate, arylphosphonate, C₁₋₆alkylarylphosphonate, C₂₋₆alkenylarylphosphonate, C₂₋₆alkynylarylphosphonate. In one embodiment, the phosphorus-containing group is selected from the group consisting of phosphate, phosphonate, C₁₋₆alkylphosphate, and C₁₋₆alkylphosphonate.

In another embodiment, each amino acid is independently an amino acid residue of the formula (II) wherein:
R¹ and R³ are each hydrogen;
R² is selected from the group consisting of C₁₋₆alkyl, C₁₋₆alkylaryl, and C₁₋₆alkylheteroaryl, wherein each C₁₋₆alkyl is substituted with hydroxyl, thiol, amino, amido, carboxyl, or guanidino, and each C₁₋₆alkylaryl is substituted with hydroxyl; and
m is 0 and p is 0.

In one embodiment, each phosphorus-containing group is bound to R².

In one embodiment, the phosphorus-containing group is selected from the group consisting of phosphate, C₁₋₆alkylphosphate, C₂₋₆alkenylphosphate, C₂₋₆alkynylphosphate, arylphosphate, C₁₋₆alkylarylphosphate, C₂₋₆alkenylarylphosphate, C₂₋₆alkynylarylphosphate, phosphonate, C₁₋₆alkylphosphonate, C₂₋₆alkenylphosphonate, C₂₋₆alkynylphosphonate, arylphosphonate, C₁₋₆alkylarylphosphonate, C₂₋₆alkenylarylphosphonate, C₂₋₆alkynylarylphosphonate. In one embodiment, the phosphorus-containing group is selected from the group consisting of phosphate, phosphonate, C₁₋₆alkylphosphate, and C₁₋₆alkylphosphonate.

In one embodiment, each amino acid is independently a natural amino acid; or an unnatural amino acid residue of the formula (II) wherein:
R¹ and R³ are each hydrogen;
R² is C₁₋₆alkylheteroaryl; and
m is 0 and p is 0.

In one embodiment, each phosphorus-containing group is bound to the oxygen atom of a hydroxyl group in a serine, threonine, or tyrosine residue; the nitrogen atom of an imidazole ring in a histidine residue; or the heteroaryl group of an amino acid residue of the formula (II) wherein R² is C₁₋₆alkylheteroaryl.

In one embodiment, each phosphorus-containing group is bound to the oxygen atom of a hydroxyl group in a serine, threonine, or tyrosine residue; or the heteroaryl group of an amino acid residue of the formula (II) wherein R² is C₁₋₆alkylheteroaryl.

In one embodiment, the heteroaryl group is a triazole ring. In one embodiment, the triazole ring is a 1,2,3-triazole ring. In one embodiment, the 1,2,3-triazole ring is 1,4-substituted.

In one embodiment, each phosphorus-containing group bound to the oxygen atom of a hydroxyl group in a serine, threonine, or tyrosine residue is a —P(O)(OH)₂ group.

In one embodiment, each phosphorus-containing group bound to the heteroaryl group of an amino acid residue of the formula (II) wherein R² is C₁₋₆alkylheteroaryl is a C₁₋₆alkylphosphonate. In one embodiment, the heteroaryl group is a triazole ring. In one embodiment, the triazole ring is a 1,2,3-triazole ring.

In one embodiment, the phosphopeptide comprises:

an amino acid sequence of the formula (I):

Xaa¹-Xaa²-Xaa³ₙ (I)
wherein:
n is an integer from 2 to 50;
Xaa¹, Xaa², and Xaa³ at each instance of n are each independently an amino acid residue of the formula (II):

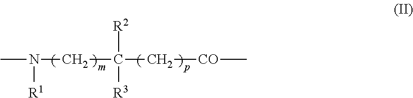

wherein:
   R¹ is selected from the group consisting of hydrogen, C₁₋₆alkyl, C₂₋₆alkenyl, and C₂₋₆alkynyl, each of which is optionally substituted with one or more substituents independently selected from the group consisting of hydroxyl, C₁₋₆alkyoxy, thiol, C₁₋₆alkylthio, halo, C₁₋₆haloalkoxy, cyano, nitro, amino, and carboxyl;
   R² is selected from the group consisting of hydrogen, C₁₋₆alkyl, C₂₋₆alkenyl, C₂₋₆alkynyl, C₃₋₁₀cycloalkyl, C₁₋₆alkylC₃₋₁₀cycloalkyl, C₂₋₆alkenylC₃₋₁₀cycloalkyl, C₂₋₆alkynylC₃₋₁₀cycloalkyl, C₃₋₁₀cycloalkenyl, C₁₋₆alkylC₃₋₁₀cycloalkenyl, C₂₋₆alkenylC₃₋₁₀cycloalkenyl, C₂₋₆alkynylC₃₋₁₀cycloalkenyl, aryl, C₁₋₆alkylaryl, C₂₋₆alkenylaryl, C₂₋₆alkynylaryl, heteroaryl, C₁₋₆alkylheteroaryl, C₂₋₆alkenylheteroaryl, C₂₋₆alkynylheteroaryl, heterocyclyl, C₁₋₆alkylheterocyclyl, C₂₋₆alkenylheterocyclyl, and C₂₋₆alkynylheterocyclyl, each of which is optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, acyl, amino, amido, acylamino, carboxyl, acyloxy, guanidino, urea, carbonate, thiourea, cyano, nitro, nitroso, azide, cyanate, thiocyanate, and isocyanate;
   R³ is selected from the group consisting of hydrogen, C₁₋₆alkyl, C₂₋₆alkenyl, and C₂₋₆alkynyl, each of which is optionally substituted with one or more substituents independently selected from the group consisting of hydroxyl, C₁₋₆alkyoxy, thiol, C₁₋₆alkylthio, halo, C₁₋₆haloalkoxy, cyano, nitro, amino, and carboxyl;
   or R¹ and R² together with nitrogen atom and carbon atom to which they are attached form a 5- or 6-membered heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, cyano, and nitro;
   or R² and R³ together with the carbon atom to which they are attached form a 5- or 6-membered cycloalkyl, cycloalkenyl, or heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, acyl, amino, amido, acylamino, carboxyl, acyloxy, guanidino, urea, carbonate, thiourea, cyano, nitro, nitroso, azide, cyanate, thiocyanate, and isocyanate;
   m is an integer from 0 to 2 and p is 0, or m is 0 and p is an integer from 0 to 2; and
provided that Xaa¹, Xaa², and Xaa³ respectively, at each instance of n, have similar structural and/or electronic properties;
   two or more phosphorus-containing groups capable of interacting with the surface of the metal nanoparticle,
wherein each phosphorus-containing group is bound to R², the optionally substituted ring formed when R¹ and R² are taken together with nitrogen atom and carbon atom to which they are attached, or the optionally substituted ring formed when R² and R³ are taken together with the carbon atom to which they are attached of an amino acid in the amino acid sequence of formula (I); and
   optionally a group that mitigates aggregation of the metal nanoparticle-phosphopeptide complex with metal nanoparticles or other metal nanoparticle-phosphopeptide complexes;
wherein each group that mitigates aggregation is bound to R², the optionally substituted ring formed when R¹ and R² are taken together with nitrogen atom and carbon atom to which they are attached, or the optionally substituted ring formed when R² and R³ are taken together with the carbon atom to which they are attached of an amino acid in the amino acid sequence of formula (I).

In one embodiment, and Xaa¹, Xaa², or Xaa³ that is bound to a phosphorus-containing group is not adjacent to another Xaa¹, Xaa², or Xaa³ that is bound to a phosphorus-containing group.

In one embodiment, Xaa¹, Xaa², and Xaa³, respectively, at each instance of n have similar structural and electronic properties.

In one embodiment, the phosphorus-containing group is selected from the group consisting of phosphate, phosphonate, C₁₋₆alkylphosphate, and C₁₋₆alkylphosphonate. In one embodiment, the phosphorus-containing group is phosphate or phosphonate.

In one embodiment, the group that mitigates aggregation is selected from the group consisting of sulfate, C₁₋₆alkylsulfate, sulfonate, C₁₋₆alkylsulfonate, poly(ethylene oxide), poly(betaine), poly(saccharide), and a charged peptide. In another embodiment, the group that mitigates aggregation is selected from the group consisting of sulfate, C₁₋₆alkylsulfate, sulfonate, and C₁₋₆alkylsulfonate.

In one embodiment, one or more of the amino acids of each peptide motif are natural amino acids. In another embodiment, two or more of the amino acids of each peptide motif are natural amino acids. In one embodiment, the amino acid is N- or O-bound to a phospho group.

In another embodiment, one or more of the amino acids of each peptide motif are natural amino acids. In another embodiment, two or more of the amino acids of each peptide motif are natural amino acids. In another embodiment, all of the amino acids of each peptide motif are natural amino acids; or all of the amino acids of each peptide motif are natural amino acids, except any amino acid bound to a phosphorus containing group. In another embodiment, all of the amino acids of each peptide motif are natural amino acids.

In a further aspect, the present invention provides a phosphopeptide as defined in any of the embodiments described herein.

In a further aspect, the present invention provides a composition comprising a plurality of metal nanoparticles and a phosphopeptide of the present invention. In one embodiment, the metal is as defined in any of the preceding embodiments.

In a further aspect, the present invention provides a composition comprising a plurality of metal nanoparticle-phosphopeptide complexes of the present invention. In one embodiment, the metal is as defined in any of the preceding embodiments.

In one embodiment, the composition further comprises a solvent in which the metal nanoparticle-phosphopeptide complexes are suspended. In one embodiment, the suspension is stable for at least one day. In one embodiment, the solvent is water or an alcohol. In one embodiment, the alcohol is ethanol. In one embodiment, the solvent is water.

In another embodiment, the composition further comprises a pharmaceutically acceptable carrier, excipient, or diluent.

In a further aspect, the present invention provides a method for preparing metal nanoparticles, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs;
      in a liquid reaction medium under conditions that form metal nanoparticles.

In a further aspect, the present invention provides a method for preparing a metal nanoparticle-phosphopeptide complex, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs;
      in a liquid reaction medium under conditions that form a metal nanoparticle-phosphopeptide complex.

In one embodiment, the metal is as defined in any of the preceding embodiments.

In one embodiment, the metal compound comprises a metal cation.

In one embodiment, the method comprises contacting two or more metal compounds. In one embodiment, the at least two of the two or more metal compounds comprise different metals.

In one embodiment, the method comprises reducing the metal compound with a reducing agent in the presence of the phosphopeptide complex to form the metal nanoparticle-phosphopeptide complex.

In another embodiment, the method comprises precipitating metal nanoparticles from the metal compound in the presence of the phosphopeptide to form the metal nanoparticle-phosphopeptide complex.

In a further aspect, the present invention provides a method for preparing metal nanoparticles, the method comprising contacting
a metal compound;
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
a reducing agent
   in a liquid reaction medium to form metal nanoparticles.

In one embodiment, the metal is as defined in any of the preceding embodiments.

In a further aspect, the present invention provides a method for preparing a metal nanoparticle-phosphopeptide complex, the method comprising contacting
a metal compound;
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
a reducing agent
   in a liquid reaction medium to form a metal nanoparticle-phosphopeptide complex.

In one embodiment, the metal is as defined in any of the preceding embodiments.

In one embodiment, the metal compound is a metal salt.

In one embodiment the metal salt is an iron (II), iron (III), platinum (II), palladium (II), ruthenium (II), ruthenium (III), silver (I), iridium (III), rhodium (III), gold (III), copper (II), cobalt (III), or nickel (II) salt. In one embodiment the metal compound is FeSO₄, Pt(NH₃)₄(NO₃)₂, PdCl₂, RuCl₃, Ag(CF₃COO), IrCl₃, RhCl₃, AuCl₃, Cu(OAc)₂, CoCl₃, Ni(OAc)₂.

The phosphopeptide is as defined in any of the embodiments described herein. In one embodiment, the molar concentration of phosphopeptide relative to metal is low.

In one embodiment, the molar ratio of metal to phosphopeptide is more than about 1:1, 2:1, 3:1, 4:1, 5:1, 6:1, 7:1, 8:1, 9:1, 10:1, 15:1, or 20:1. In one embodiment, the molar ratio is from about 1:1 to 25:1, 1:1 to 20:1, 1:1 to 15:1, 1:1 to 10:1, 1:1 to 5:1, 5:1 to 25:1, 5:1 to 20:1, 5:1 to 15:1, 5:1 to 10:1, 10:1 to 25:1, 10:1 to 20:1, 10:1 to 15:1, 15:1 to 25:1, 15:1 to 20:1, or 20:1 to 25:1.

In one embodiment, the reducing agent is a metal hydride. In one embodiment, the metal hydride is a metal borohydride. In one embodiment, the metal borohydride is sodium borohydride.

In one embodiment, the methods comprise mixing the metal compound, phosphopeptide, and reducing agent in the liquid reaction medium.

In one embodiment, the methods further comprise recovering the product metal nanoparticles or metal nanoparticle-phosphopeptide complex.

In one embodiment, the nanoparticle is an iron nanoparticle. In one embodiment, the iron nanoparticle is an iron-iron oxide core-shell nanoparticle. In one embodiment, the size of the iron-iron oxide core-shell nanoparticle is from about 8 nm to about 25 nm. In another embodiment, the size of the iron-iron oxide core-shell nanoparticle is from about 15 nm to about 25 nm.

In another embodiment, the iron nanoparticle is an iron oxide nanoparticle. In one embodiment, the size of the iron oxide nanoparticle is about 8 nm.

In one embodiment, the metal nanoparticle is a cobalt nanoparticle.

In one embodiment, the metal nanoparticle is a nickel nanoparticle.

In one embodiment, the metal nanoparticle is a copper nanoparticle.

In one embodiment, the metal nanoparticle is a ruthenium nanoparticle. In one embodiment, the size of the ruthenium nanoparticle is from about 20 nm to 100 nm.

In one embodiment, the metal nanoparticle is a rhodium nanoparticle.

In one embodiment, the metal nanoparticle is a palladium nanoparticle. In one embodiment, the size of the palladium nanoparticle is from about 3 nm to about 7 nm. In another embodiment, the size of the palladium nanoparticle is about 5 nm.

In one embodiment, the metal nanoparticle is a silver nanoparticle.

In one embodiment, the metal nanoparticle is an iridium nanoparticle.

In one embodiment, the metal nanoparticle is a platinum nanoparticle.

In one embodiment, the metal nanoparticle is a gold nanoparticle. In one embodiment, the size of the gold nanoparticle is from about 3 nm to about 5 nm. In another embodiment, the size of the gold nanoparticle is about 4 nm.

In one embodiment, the metal nanoparticles are substantially monodisperse.

In one embodiment, the liquid reaction medium is water.

In one embodiment, the methods are carried out at ambient temperature.

In one embodiment, the reaction is carried out for a period of time from 2 minutes to 12 hours, 2 minutes to 3 hours, 2 minutes to 1 hour, 5 minutes to 12 hours, 5 minutes to 3 hours, 5 minutes to 1 hour, 10 minutes to 12 hours, 10 minutes to 3 hours, 10 minutes to 1 hour.

In a further aspect, the present invention provides a method for preparing metal nanoparticles, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
      in a liquid reaction medium under conditions that precipitate metal nanoparticles.

In a further aspect, the present invention provides a method for preparing a metal nanoparticle-phosphopeptide complex, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
      in a liquid reaction medium under conditions that precipitate a metal nanoparticle-phosphopeptide complex.

In one embodiment, the metal is as defined in any of the preceding embodiments.

In one embodiment, the method comprises contacting two or more metal compounds. In one embodiment, at least two of the two or more metal compounds comprise different metals.

In one embodiment, the method comprises co-precipitating two or more metal compounds in the presence of the phosphopeptide to form the metal nanoparticle phosphopeptide complex.

In one embodiment, at least one of the metal compounds comprises iron.

In one embodiment, the metal compound is a metal salt. In one embodiment, the metal salt is as defined in any of the preceding embodiments.

The phosphopeptide is as defined in any of the embodiments described herein. In one embodiment, the molar concentration of phosphopeptide relative to metal is low.

In one embodiment, the molar ratio of metal to phosphopeptide is more than about 1:1, 2:1, 3:1, 4:1, 5:1, 6:1, 7:1, 8:1, 9:1, 10:1, 15:1, or 20:1. In one embodiment, the molar ratio is from about 1:1 to 25:1, 1:1 to 20:1, 1:1 to 15:1, 1:1 to 10:1, 1:1 to 5:1, 5:1 to 25:1, 5:1 to 20:1, 5:1 to 15:1, 5:1 to 10:1, 10:1 to 25:1, 10:1 to 20:1, 10:1 to 15:1, 15:1 to 25:1, 15:1 to 20:1, or 20:1 to 25:1.

In one embodiment, the methods comprise mixing the metal compound and phosphopeptide in the liquid reaction medium.

In one embodiment, the methods further comprise recovering the product metal nanoparticles or metal nanoparticle-phosphopeptide complex.

In one embodiment, the metal nanoparticle is a metal oxide, metal hydroxide, or metal chalcogenide nanoparticle.

In one embodiment, the method comprises contacting one or more metal compounds, a phosphopeptide, and hydroxide or chalcogen anions in the liquid reaction medium. In one embodiment, the chalcogen is sulfur.

In one embodiment, the liquid reaction medium comprises water. In one embodiment, the liquid reaction medium is water.

In one embodiment, the liquid reaction medium comprises base.

In one embodiment, the metal nanoparticles are substantially monodisperse.

In one embodiment, the methods are carried out at ambient temperature.

In a further aspect, the present invention provides a method for preparing iron nanoparticles, the method comprising contacting
iron (II);
iron (III);
a phosphopeptide comprising two or more contiguous peptide motifs and two or more phosphorus-containing groups capable of interacting with the surface of the iron nanoparticle,
   wherein the amino acids at the equivalent position in each peptide motif have similar structural and/or electronic properties, and
   wherein each phosphorus-containing group is bound to an amino acid in the two or more contiguous peptide motifs; and
a base
   in a liquid reaction medium to form iron nanoparticles.

In a further aspect, the present invention provides a method for preparing an iron nanoparticle-phosphopeptide complex, the method comprising contacting
iron (II);
iron (III);
a phosphopeptide comprising two or more contiguous peptide motifs and two or more phosphorus-containing groups capable of interacting with the surface of the iron nanoparticle,
   wherein the amino acids at the equivalent position in each peptide motif have similar structural and/or electronic properties, and
   wherein each phosphorus-containing group is bound to an amino acid in the two or more contiguous peptide motifs; and
a base
   in a liquid reaction medium to provide an iron nanoparticle-phosphopeptide complex.

In one embodiment, iron (II) is provided to the liquid reaction mixture in the form of an iron (II) compound. In one embodiment, iron (III) is provided to the liquid reaction mixture in the form of an iron (III) compound. In one embodiment iron (II) and iron (III) are provided to the liquid reaction mixture in the form of an iron (II) compound and an iron (III) compound, respectively.

In one embodiment, the iron (II) compound is an iron (II) salt. In one embodiment, the iron (III) compound is an iron (III) salt. In one embodiment, the iron (II) compound is an iron (II) salt and the iron (III) compound is an iron (III) salt.

In one embodiment, the iron (II) compound is iron (II) sulfate and the iron (III) compound is iron (III) chloride.

In one embodiment, the base is ammonia.

The phosphopeptide is as defined in any of the embodiments described herein. In one embodiment, the molar ratio of phosphorus-containing groups to iron is less than 1:1. In another embodiment, the molar ratio of phosphorus-containing groups to iron (III) is less than 1:1.

In one embodiment, the methods comprise mixing the iron (II), iron (III), phosphopeptide, and base in the liquid reaction medium.

In one embodiment, the methods further comprise recovering the product iron nanoparticles or iron nanoparticle-phosphopeptide complex.

In one embodiment, the iron nanoparticle is an iron oxide nanoparticle. In one embodiment, the iron oxide nanoparticle is about 5 nm.

In one embodiment, the iron nanoparticles are substantially monodisperse.

In one embodiment, the liquid reaction medium comprises water. In one embodiment, the liquid reaction medium is water.

In one embodiment, the methods are carried out at ambient temperature.

In one embodiment, the reaction is carried out for a period of time from 2 minutes to 12 hours, 2 minutes to 3 hours, 2 minutes to 1 hour, 5 minutes to 12 hours, 5 minutes to 3 hours, 5 minutes to 1 hour, 10 minutes to 12 hours, 10 minutes to 3 hours, 10 minutes to 1 hour.

In a further aspect, the present invention provides metal nanoparticles prepared by a method of the present invention.

In a further aspect, the present invention provides a metal nanoparticle-phosphopeptide complex prepared by a method of the present invention.

In a further aspect, the present invention provides a use of a metal nanoparticle-phosphopeptide complex of the present invention in the manufacture of a medicament for treating cancer. In a further aspect, the present invention provides a use of a metal nanoparticle-phosphopeptide complex of the present invention in the manufacture of a contrast agent for contrast enhancement in medical imaging.

In a further aspect, the present invention provides a metal nanoparticle-phosphopeptide complex of the present invention for use in treating cancer. In a further aspect, the present invention provides a metal nanoparticle-phosphopeptide complex of the present invention for use in contrast enhancement in medical imaging.

In a further aspect, the present invention provides a method of treating cancer comprising administering an effective amount of a metal nanoparticle-phosphopeptide complex of the present invention to a patient in need thereof, and applying an alternating magnetic field to heat the nanoparticles. In a further aspect, the present invention provides a method of imaging comprising administering an effective amount of a metal nanoparticle-phosphopeptide complex of the present invention to a patient in need thereof, and imaging the patient.

In a further aspect, the present invention provides a use of the metal nanoparticles of the present invention in the manufacture of a medicament for treating cancer. In a further aspect, the present invention provides a use of the metal nanoparticles of the present invention in the manufacture of a contrast agent for contrast enhancement in medical imaging.

In a further aspect, the present invention provides metal nanoparticles of the present invention for use in treating cancer. In a further aspect, the present invention provides metal nanoparticles for use in contrast enhancement in medical imaging.

In a further aspect, the present invention provides a method of treating cancer comprising administering an effective amount of metal nanoparticles of the present invention to a patient in need thereof, and applying an alternating magnetic field to heat the nanoparticles. In a further aspect, the present invention provides a method of imaging comprising administering an effective amount of metal nanoparticles of the present invention to a patient in need thereof, and imaging the patient.

In a further aspect, the present invention provides a kit for preparing a therapeutic or diagnostic agent comprising:
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs.

In one embodiment, the kit further comprises instructions for preparing a metal nanoparticle-phosphopeptide complex by a method of the present invention.

In another embodiment, the kit further comprises a reducing agent. In one embodiment, the reducing agent is sodium borohydride.

In one embodiment, the kit comprises a liquid medium in which the metal nanoparticle-phosphopeptide complex is prepared.

In a further aspect, the present invention provides a kit for preparing a therapeutic or diagnostic agent comprising:
a metal nanoparticle-phosphopeptide complex of the present invention.

In one embodiment, the kit comprises a liquid medium in which the metal nanoparticle-phosphopeptide complex is suspended.

In one embodiment, the therapeutic agent is for use in treating cancer. In one embodiment, the diagnostic agent is for use as a contrast agent in medical imaging.

In another embodiment, the kits further comprise a compound that minimises non-specific interactions and/or inflammatory reactions in vivo. In one embodiment, the kit further comprises instructions for coupling the compound to the metal nanoparticle-phosphopeptide complex.

In one embodiment, the kits comprise a targeting group that has a specific interaction with a target in vivo. In one embodiment, the targeting group comprises an antibody that has a specific interaction with a target antigen in vivo. In one embodiment, the target antigen is a cell-surface receptor.

In one embodiment, the kits further comprise an activating agent to facilitate coupling of the compound and/or targeting group to the metal nanoparticle-phosphopeptide complex. In one embodiment, the activating agent is an activating agent for peptide coupling. In one embodiment, the kit further comprises instructions for coupling the compound and/or targeting group to the metal nanoparticle-phosphopeptide complex. In one embodiment, the kits comprise a liquid medium in which the coupling reaction(s) are carried out.

In one embodiment, the metal nanoparticle comprises iron, cobalt, nickel, or a mixture of any two or more thereof. In another embodiment, the metal nanoparticle comprises iron or a mixture of iron and cobalt, iron and nickel, or iron, cobalt, and nickel. In another embodiment, the metal nanoparticle comprises iron. In another embodiment, the metal nanoparticle is an iron nanoparticle.

In a further aspect, the present invention provides a metal nanoparticle-phosphopeptide complex of the present invention for use as a catalyst.

In a further aspect, the present invention provides a catalyst comprising a metal nanoparticle-phosphopeptide complex of the present invention.

In a further aspect, the present invention provides use of a metal nanoparticle-phosphopeptide complex of the present invention as a catalyst.

As used herein the term “and/or” means “and”, or “or”, or both.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

As used herein the “nanoparticle” refers to any particle less than 1000 nanometres in size. In one embodiment, the nanoparticle is less than 1000 nm, 750 nm, 500 nm, 450 nm, 400 nm, 350 nm, 300 nm, 250 nm, 200 nm, 175 nm, 150 nm, 125 nm, or 100 nm. In one embodiment, the nanoparticle is less than 100 nm. Those persons skilled in the art will appreciate that references to nanoparticles in this specification it also include nanocrystals, even though a nanocrystal may have a higher degree of crystallinity than a nanoparticle.

The term “metal nanoparticle” as used herein refers to a nanoparticle that comprises metal. In one embodiment, the metal nanoparticle comprises metal, metal oxide, metal hydroxide, metal chalcogenide, or a mixture of metal and metal oxide, metal and metal hydroxide, metal and metal chalcogenide, or metal, metal oxide, and metal hydroxide. In another embodiment, the metal nanoparticle is substantially composed of metal, metal oxide, metal hydroxide, metal chalcogenide, or a mixture of metal and metal oxide, metal and metal hydroxide, metal and metal chalcogenide, or metal, metal oxide, and metal hydroxide. In one embodiment, the chalcogen is sulfur, selenium, or tellurium. In one embodiment, the chalcogen is sulfur. In one embodiment, the metal nanoparticle is substantially composed of metal, metal oxide, metal hydroxide, or a mixture of metal and metal oxide, metal and metal hydroxide, or metal, metal oxide, and metal hydroxide. In another embodiment, the metal nanoparticle is substantially composed of metal, metal oxide, or a mixture of metal and metal oxide.

A metal nanoparticle may have a “core” comprising metal surrounded by a “shell” comprising metal oxide. The term “core” refers to the central region of the nanoparticle. A core can substantially include a single homogeneous material. A core may be crystalline or amorphous. Whilst a core may be referred to as crystalline, it is understood that the surface of the core may be amorphous or polycrystalline and that this non-crystalline surface layer may extend a finite depth into the core.

The term “metal nanoparticle-phosphopeptide complex” as used herein refers to a metal nanoparticle having one or more phosphopeptides on its surface. The phosphopeptide may be adsorbed on the surface of the metal nanoparticle. The phosphopeptide may also be partially incorporated into the surface of the metal nanoparticle.

As used herein the “size” of a nanoparticle refers to the diameter of the nanoparticle.

The term “peptide” as used herein alone or in combination with other terms means a chain of two or more natural or unnatural amino acids joined by a peptide bond.

The term “phosphopeptide” as used herein alone or in combination with other terms means a peptide that comprises one or more phosphorus-containing groups. In one embodiment, the phosphorus containing group is capable of interacting with the surface of a metal nanoparticle. In one embodiment, the phosphopeptide comprises from 4 to 500 amino acids. In another embodiment, the phosphopeptide comprises from 4 to 300 amino acids. In one embodiment, the phosphopeptide comprises from 6 to 300 amino acids. In another embodiment, the phosphopeptide comprises from 6 to 150 amino acids. In another embodiment, the phosphopeptide comprises from 6 to 100 amino acids. In another embodiment, the phosphopeptide comprises from 6 to 75 amino acids. In another embodiment, the phosphopeptide comprises from 6 to 50 amino acids. In another embodiment, the phosphopeptide comprises from 6 to 25 amino acids. In another embodiment, the phosphopeptide comprises from 6 to 75, from 6 to 70, from 6 to 65, from 6 to 60, from 6 to 55, from 6 to 50, from 6 to 45, from 6 to 40, from 6 to 35, from 6 to 30, from 6 to 25, from 6 to 20, or from 6 to 18 amino acids.

The term “phosphate” employed alone or in combination with other terms means, unless otherwise stated, a —OP(O)(OR¹)(OR²) group, wherein R¹ and R² are each independently selected from the group consisting of hydrogen and a metal cation.

The term “phosphonate” employed alone or in combination with other terms means, unless otherwise stated, a —P(O)(OR¹)(OR²) group, wherein Wand R² are each independently selected from the group consisting of hydrogen and a metal cation.

The term “pyrophosphate” employed alone or in combination with other terms means, unless otherwise stated, a —OP(O)(OR¹)OP(O)(OR²)(OR³) group, wherein R¹, R², and R³ are each independently selected from the group consisting of hydrogen and a metal cation.

The term “sulfate” employed alone or in combination with other terms means, unless otherwise stated, a —OS(O)₂OR group, wherein R is selected from the group consisting of hydrogen and a metal cation.

The term “sulfonate” employed alone or in combination with other terms means, unless otherwise stated, a —S(O)₂OR group, wherein R is selected from the group consisting of hydrogen and a metal cation.

The term “alkyl” employed alone or in combination with other terms means, unless otherwise stated, a monovalent straight chain or branched chain saturated hydrocarbon group. In one embodiment, alkyl groups comprise 1 to 6 carbon atoms. Examples of alkyl groups include methyl, ethyl, n-propyl, iso-propyl, n-butyl, tert-butyl, sec-butyl, iso-butyl, n-pentyl, neopentyl, iso-pentyl, tert-pentyl, sec-pentyl, n-hexyl, neohexyl, iso-hexyl, tert-hexyl, sec-hexyl, and the like.

The term “alkenyl” employed alone or in combination with other terms means, unless otherwise stated, a monovalent straight chain or branched chain hydrocarbon group including one or more carbon-carbon double bonds. In one embodiment, alkenyl groups comprise 2 to 6 carbon atoms. Examples of alkenyl groups include vinyl, prop-2-enyl, crotyl, isopent-2-enyl, 2-butadienyl, penta-2,4-dienyl, penta-1,4-dienyl, and the like.

The term “alkynyl” employed alone or in combination with other terms means, unless otherwise stated, a monovalent straight chain or branched chain hydrocarbon group including one or more carbon-carbon triple bonds. In one embodiment, alkynyl groups comprise 2 to 6 carbon atoms. Examples of alkynyl groups include ethynyl, prop-3-ynyl, but-3-ynyl, and the like.

The term “cycloalkyl”, employed alone or in combination with other terms means, unless otherwise stated, a monovalent saturated cyclic hydrocarbon group. In one embodiment, cycloalkyl groups contain from 3 to 10 ring carbon atoms. In another embodiment, cycloalkyl groups comprise from 3 to 8 ring carbon atoms. Examples of cycloalkyl groups include cyclopropyl, cyclobutyl, cyclopentyl, cyclohexyl, cycloheptyl, cyclooctyl, and the like.

The term “cycloalkenyl”, employed alone or in combination with other terms means, unless otherwise stated, a non-aromatic monovalent cyclic hydrocarbon group containing one or more carbon-carbon double bonds. In one embodiment, cycloalkenyl groups contain from 3 to 10 ring carbon atoms. In another embodiment, cycloalkenyl groups comprise 3 to 8 ring carbon atoms. Examples of cycloalkenyl groups include, but are not limited to, cyclopropenyl, cyclobutenyl, cyclopentenyl, cyclohexenyl, cycloheptentyl, cyclooctenyl, and the like.

The term “aryl” employed alone or in combination with other terms means, unless otherwise stated, a phenyl ring or a monovalent bicyclic or tricyclic aromatic ring system comprising only carbon and hydrogen atoms. Monovalent bicyclic aromatic ring systems include naphthyl groups and phenyl rings fused to cycloalkyl rings. Examples of monovalent bicyclic aromatic ring systems include dihydroindenyl, indenyl, naphthyl, dihydronaphthalenyl, tetrahydronaphthalenyl, and the like. Monovalent tricyclic aromatic ring systems include anthracenyl groups, phenanthrenyl groups, and monovalent bicyclic aromatic rings system fused to cycloalkyl or phenyl rings. Examples of monovalent tricyclic aromatic ring systems include azulenyl, dihydroanthracenyl, fluorenyl, tetrahydrophenanthrenyl, and the like.

The term “heteroaryl” employed alone or in combination with other terms means, unless otherwise stated, a monocyclic heteroaryl group or bicyclic heteroaryl group. Monocyclic heteroaryl groups include monovalent 5- or 6-membered aromatic rings containing at least one ring heteroatom independently selected from the group consisting of nitrogen, oxygen, and sulfur. Examples of 5- and 6-membered heteroaryl rings include furyl, imidazolyl, isoxazolyl, isothiazolyl, oxazolyl, pyrazinyl, pyrazolyl, pyridazinyl, pyridinyl, pyrimidinyl, pyrrolyl, tetrazolyl, thiadiazolyl, thiadiazolonyl, thiadiazinonyl, oxadiazolyl, oxadiazolonyl, oxadiazinonyl, thiazolyl, thienyl, triazinyl, triazolyl, pyridazinonyl, pyridinyl, pyrimidinonyl, and the like. Bicyclic heteroaryl groups include monovalent 8-, 9-, 10-, 11-, or 12-membered bicyclic aromatic rings containing one or more ring heteroatoms independently selected from the group consisting of oxygen, sulfur, and nitrogen. Examples of bicyclic heteroaryl rings include indolyl, benzothienyl, benzofuranyl, indazolyl, benzimidazolyl, benzothiazolyl, benzoxazolyl, benzoisothiazolyl, benzoisoxazolyl, quinolinyl, isoquinolinyl, quinazolinyl, quinoxalinyl, phthalazinyl, pteridinyl, purinyl, naphthyridinyl, pyrrolopyrimidinyl, and the like.

The term “heterocyclyl” employed alone or in combination with other terms means, unless otherwise stated, a saturated or unsaturated non-aromatic monocyclic heterocyclyl ring or a bicyclic heterocyclyl ring. Monocyclic heterocyclyl rings include monovalent 3-, 4-, 5-, 6-, or 7-membered rings containing one or more ring heteroatoms independently selected from the group consisting of oxygen, nitrogen, and sulfur. Examples of monocyclic heterocyclyl groups include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiopyranyl, trithianyl, and the like. Bicyclic heterocyclyl rings include monovalent monocyclic heterocyclyl rings fused to phenyl rings, cycloalkyl rings, or other monocyclic heterocyclyl rings. Examples of bicyclic heterocyclyl groups include, but are not limited to, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, 1,2,3,4-tetrahydroquinolinyl, and the like.

The term “aliphatic” employed alone or in combination with other terms means, unless otherwise stated, straight chain or branched chain saturated or unsaturated hydrocarbon group. Those skilled in the art will appreciate that aliphatic groups include alkyl, alkenyl, and alkynyl groups.

The term “heteroaliphatic” employed alone or in combination with other terms means, unless otherwise stated, means an aliphatic group wherein one or more of the carbon atoms in the main hydrocarbon chain are replaced with heteroatoms independently selected from the group consisting of oxygen, nitrogen, and sulfur. Examples of heteroaliphatic groups include alkoxyalkyl and alkylthioalkyl groups, and the like.

The term “alicyclic” employed alone or in combination with other terms means, unless otherwise stated, means a non-aromatic cyclic aliphatic group. Those skilled in the art will appreciate that alicyclic groups include cycloalkyl and cycloalkenyl groups.

The term “heteroalicyclic” employed alone or in combination with other terms means, unless otherwise stated, means an alicyclic group wherein one or more of the carbon atoms in the ring are replaced with heteroatoms independently selected from the group consisting of oxygen, nitrogen, and sulfur. Those skilled in the art will appreciate that heteroalicyclic groups include heterocyclyl groups. Examples of heteroalicyclic groups include oxazolidinyl, piperidinyl, pyrrolidinyl and tetrahydrofuranyl groups, and the like.

The term “amino” employed alone or in combination with other terms means, unless stated otherwise, a —NR¹R² group, wherein R¹ and R² are each independently selected from the group consisting of hydrogen, C₁₋₆ alkyl, and aryl.

The term “amido” employed alone or in combination with other terms means, unless stated otherwise, an amino-C(O)— group, wherein amino is as defined herein.

The term “acylamino” employed alone or in combination with other terms means, unless stated otherwise, a R¹C(O)NR²— group, wherein R¹ and R² are each independently selected from the group consisting of hydrogen, alkyl, and aryl.

The term “carboxyl” employed alone or in combination with other terms means, unless stated otherwise, a R¹C(O)O— group, wherein R¹ is selected from the group consisting of hydrogen, alkyl, aryl, and a metal cation.

The term “acyloxy” employed alone or in combination with other terms means, unless stated otherwise, a R¹C(O)O— group, wherein R¹ is selected from the group consisting of hydrogen, alkyl, and aryl.

The term “guanidino” employed alone or in combination with other terms means, unless stated otherwise, an amino-C(NR¹)NR²— group, wherein amino is as defined herein and R¹ and R² are each independently selected from the group consisting of hydrogen, alkyl, and aryl.

The term “urea” employed alone or in combination with other terms means, unless stated otherwise, an amino-C(O)—NR¹— group, wherein amino is as defined herein and R¹ is selected from the group consisting of hydrogen, alkyl, and aryl.

The term “carbonate” employed alone or in combination with other terms means, unless stated otherwise, a carboxyl-O— group, wherein carboxyl is as defined herein.

The term “thiourea” employed alone or in combination with other terms means, unless stated otherwise, an amino-C(S)—NR¹— group, wherein amino is as defined herein and R¹ is selected from the group consisting of hydrogen, alkyl, and aryl.

As used herein, the term “substituted” is intended to mean that one or more hydrogen atoms in the group indicated is replaced with one or more independently selected suitable substituents, provided that the normal valency of each atom to which the substituent(s) are attached is not exceeded, and that the substitution results in a stable compound.

The other general chemical terms used in the formulae herein have their usual meanings.

Asymmetric centers exist in the phosphopeptide. The asymmetric centers may be designated by the symbols R or S, depending on the configuration of substituents in three dimensional space at the chiral atom. All stereochemical isomeric forms of the compounds, including diastereomeric, enantiomeric, and epimeric forms, as well as D-isomers and L-isomers, erythro and threo isomers, syn and anti isomers, and mixtures thereof are contemplated herein. In one embodiment, the phosphopeptide comprises L-amino acids.

Individual enantiomers may be prepared synthetically from commercially available enantiopure starting materials or by preparing an enantiomeric mixture and resolving the mixture into individual enantiomers. Resolution methods include conversion of the enantiomeric mixture into a mixture of diastereomers and separation of the diastereomers by, for example, recrystallization or chromatography; direct separation of the enantiomers on chiral chromatographic columns; or any other appropriate method known in the art. Starting materials of defined stereochemistry may be commercially available or synthesised by techniques known in the art. In one embodiment, the starting materials include L-amino acids. In another embodiment, the starting materials include natural L-amino acids.

Geometric isomers of the phosphopeptide may also exist. All cis, trans, syn, anti, entgegen (E), and zusammen (Z) isomers, and mixtures thereof are contemplated herein.

Tautomeric isomers of the phosphopeptide, for example, keto/enol and imine/enamine tautomers, may also exist. All tautomeric isomers are contemplated herein.

Salts of the phosphopeptide, including, for example, acid addition salts, base addition salts, and quaternary salts of basic nitrogen-containing groups, are also contemplated herein.

Acid addition salts can be prepared by reacting a phosphopeptide comprising a free base with inorganic or organic acids. Examples of acid addition salts include: sulfates; methanesulfonates; acetates; hydrochlorides; hydrobromides; phosphates; toluenesulfonates; citrates; maleates; succinates; tartrates; lactates; and fumarates.

Base addition salts can be prepared by reacting a phosphopeptide comprising a free acid with inorganic or organic bases. Examples of base addition salts include: ammonium salts; alkali metal salts, for example sodium salts and potassium salts; and alkaline earth metal salts, for example calcium salts and magnesium salts. Other salts will be apparent to those skilled in the art.

Quaternary salts of basic nitrogen-containing groups can be prepared by reacting a phosphopeptide comprising a basic nitrogen-containing group with, for example, alkyl halides, for example methyl, ethyl, propyl, and butyl chlorides, bromides, and iodides; dialkyl sulfates for example dimethyl, diethyl, dibutyl, and diamyl sulfates; arylalkyl halides for example benzyl and phenylethyl bromides; and the like. Other reagents suitable for preparing quaternary salts of basic nitrogen-containing groups will be apparent to those skilled in the art.

The phosphopeptide may form or exist as solvates with various solvents. If the solvent is water, the solvate may be referred to as a hydrate, for example, a mono-hydrate, a di-hydrate, or a tri-hydrate. All solvated forms and unsolvated forms are contemplated herein.

Isotopologues and isotopomers of the phosphopeptide, wherein one or more atoms in the phosphopeptide are replaced with different isotopes, are also contemplated herein. Suitable isotopes include, for example, ¹H, ²H (D), ³H (T), ¹²C, ¹³C, ¹⁴C, ¹⁶O, and ¹⁸O.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described with reference to the Figures in which:

FIG. 1 is a transmission electron micrograph of iron nanoparticles prepared by reducing iron (II) sulfate with sodium borohydride in the absence of an additive;

FIG. 2 is a transmission electron micrograph of iron nanoparticles prepared by reducing iron (II) sulfate with sodium borohydride in the presence of sodium citrate;

FIGS. 3 and 4 are transmission electron micrographs of iron nanoparticles prepared by reducing iron (II) sulfate with sodium borohydride in the presence of 3-O-(phospho)-serine;

FIGS. 5 and 6 are transmission electron micrographs of iron nanoparticles prepared by reducing iron (II) sulfate with sodium borohydride in the presence of peptide 77;

FIGS. 7 and 8 are transmission electron micrographs of iron nanoparticles prepared by reducing iron (II) sulfate with sodium borohydride in the presence of phosphopeptide 107;

FIGS. 9 and 10 are transmission electron micrographs of iron nanoparticles prepared by reducing iron (II) sulfate with sodium borohydride in the presence of phosphopeptide 108;

FIGS. 11 and 12 are transmission electron micrographs of iron nanoparticles prepared by reducing iron (II) sulfate with sodium borohydride in the presence of phosphopeptide 109;

FIGS. 13 and 14 are transmission electron micrographs of iron nanoparticles prepared by reducing iron (II) sulfate with sodium borohydride in the presence of phosphopeptide 110;

FIGS. 15 and 16 are transmission electron micrographs of iron nanoparticles prepared by reducing iron (II) sulfate with sodium borohydride in the presence of phosphopeptide 111;

FIGS. 17 and 18 are transmission electron micrographs of iron nanoparticles prepared by coprecipitating iron (II) sulfate and iron (III) chloride with ammonia;

FIGS. 19 and 20 are transmission electron micrographs of iron nanoparticles prepared by coprecipitating iron (II) sulfate and iron (III) chloride with ammonia in the presence of phosphopeptide 107;

FIG. 21 is a magnetic hysteresis loop diagram obtained using iron nanoparticles prepared by reducing iron (II) sulfate with sodium borohydride in the presence of sodium citrate; and

FIG. 22 is a magnetic hysteresis loop diagram obtained using iron nanoparticles prepared by reducing iron (II) sulfate with sodium borohydride in the presence of phosphopeptide 107;

FIGS. 23 and 24 are transmission electron micrographs of iron nanoparticles prepared by reducing iron (II) sulfate with sodium borohydride in the presence of phosphopeptide 207;

FIGS. 25 and 26 are transmission electron micrographs of iron nanoparticles prepared by reducing iron (II) sulfate with sodium borohydride in the presence of phosphopeptide 209;

FIGS. 27 and 28 are transmission electron micrographs of iron nanoparticles prepared by reducing iron (II) sulfate with sodium borohydride in the presence of phosphopeptide 210;

FIG. 29 is a transmission electron micrograph of iron nanoparticles prepared by reducing iron (II) sulfate with sodium borohydride in the presence of phosphopeptide 303;

FIG. 30 is a transmission electron micrograph of iron nanoparticles prepared by reducing iron (II) sulfate with sodium borohydride in the presence of phosphopeptide 304;

FIG. 31 is a transmission electron micrograph of iron nanoparticles prepared by reducing iron (II) sulfate with sodium borohydride in the presence of phosphopeptide 305;

FIG. 32 is a transmission electron micrograph of iron nanoparticles prepared by reducing iron (II) sulfate with sodium borohydride in the presence of phosphopeptide 306;

FIG. 33 is histogram showing the size distribution of iron nanoparticles prepared in the presence of phosphopeptide 209 determined by dynamic light scattering;

FIG. 34 is a magnetic hysteresis loop diagram obtained using iron nanoparticles prepared in the presence of phosphopeptide 209;

FIG. 35 shows a scanning transmission electron micrograph (STEM) bright field image (a) of iron nanoparticles prepared in the presence of phosphopeptide 209, and corresponding X-ray energy dispersive spectroscopy (EDS) maps for iron (b), oxygen (c), sodium (d), nitrogen (e), and phosphorus (f);

FIGS. 36 and 37 are transmission electron micrographs of platinum nanoparticles prepared in the presence of phosphopeptide 209;

FIG. 38 is an electron diffraction pattern of platinum nanoparticles prepared in the presence of phosphopeptide 209;

FIGS. 39 and 40 are transmission electron micrographs of palladium nanoparticles prepared in the presence of phosphopeptide 209;

FIG. 41 is an electron diffraction pattern of palladium nanoparticles prepared in the presence of phosphopeptide 209;

FIGS. 42, 43, and 44 are transmission electron micrographs of ruthenium nanoparticles prepared in the presence of phosphopeptide 209;

FIG. 45 is an electron diffraction pattern of ruthenium nanoparticles prepared in the presence of phosphopeptide 209;

FIG. 46 shows a scanning transmission electron micrograph (STEM) bright field image (a) of ruthenium nanoparticles prepared in the presence of phosphopeptide 209, and corresponding X-ray energy dispersive spectroscopy (EDS) maps for ruthenium (b), oxygen (c), carbon (d), phosphorus (e), and sodium (f);

FIGS. 47, 48, and 49 are transmission electron micrographs of gold nanoparticles prepared in the presence of phosphopeptide 209; and

FIG. 50 is an electron diffraction pattern of gold nanoparticles prepared in the presence of phosphopeptide 209.

### DETAILED DESCRIPTION OF THE INVENTION

The present invention provides a metal nanoparticle-phosphopeptide complex comprising:
a metal nanoparticle; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs.

In one embodiment, the metal nanoparticle is as defined in any of the preceding embodiments.

In one embodiment, the size of the nanoparticle is from 3 to 250, 3 to 200, 3 to 150, 3 to 125, 3 to 100, 3 to 75, 3 to 50, 3 to 40, 3 to 30, 3 to 20, 3 to 10, 5 to 250, 5 to 200, 5 to 150, 5 to 125, 5 to 100, 5 to 75, 5 to 50, 5 to 40, 5 to 30, 5 to 20, 5 to 10, 7 to 250, 7 to 200, 7 to 150, 7 to 125, 7 to 100, 7 to 75, 7 to 50, 7 to 40, 7 to 30, 7 to 20, 7 to 10, 10 to 250, 10 to 200, 10 to 150, 10 to 125, 10 to 100, 10 to 75, 10 to 50, 10 to 40, 10 to 30, or 10 to 20 nm. In one embodiment, the size of the nanoparticle is less than 250, 200, 150, 125, 100, 75, 50, 40, 30, 20, 10, 7, 5, or 3 nm.

In one embodiment, the metal nanoparticle is an iron nanoparticle.

In one embodiment, the iron nanoparticle is an iron oxide nanoparticle. In one embodiment, the size of the iron oxide nanoparticle is less than about 10 nm. In another embodiment, the iron oxide nanoparticle is less than about 8 nm. In another embodiment, the size of the iron oxide nanoparticle is about 5 nm. In one embodiment, the size of the iron oxide nanoparticle is from about 5 nm to about 8 nm.

In another embodiment, the iron nanoparticle is an iron-iron oxide core-shell nanoparticle. In one embodiment, the size of the iron-iron oxide core-shell nanoparticle is less than about 50 nm. In another embodiment, the size of the iron-iron oxide core-shell nanoparticle is less than about 30 nm. In another embodiment, the size of the iron-iron oxide core-shell nanoparticle is about 20 nm. In one embodiment, the size of the iron-iron oxide core-shell nanoparticle is from about 8 nm to about 50 nm. In another embodiment, the size of the iron-iron oxide core-shell nanoparticle is from about 10 nm to about 50 nm. In another embodiment, the size of the iron-iron oxide core-shell nanoparticle is from about 8 nm to about 25 nm. In another embodiment, the size of the iron-iron oxide core-shell nanoparticle is from about 15 nm to about 25 nm.

In one embodiment, the iron nanoparticles exhibit ferromagnetic and/or ferrimagnetic behaviour at room temperature.

In one embodiment, the metal nanoparticle is a cobalt nanoparticle.

In one embodiment, the metal nanoparticle is a nickel nanoparticle.

In one embodiment, the metal nanoparticle is a copper nanoparticle.

In one embodiment, the metal nanoparticle is a ruthenium nanoparticle. In one embodiment, the size of the ruthenium nanoparticle is from about 20 nm to 100 nm.

In one embodiment, the metal nanoparticle is a rhodium nanoparticle.

In one embodiment, the metal nanoparticle is a palladium nanoparticle. In one embodiment, the size of the palladium nanoparticle is from about 3 nm to about 7 nm. In another embodiment, the size of the palladium nanoparticle is about 5 nm.

In one embodiment, the metal nanoparticle is a silver nanoparticle.

In one embodiment, the metal nanoparticle is an iridium nanoparticle.

In one embodiment, the metal nanoparticle is a platinum nanoparticle.

In one embodiment, the metal nanoparticle is a gold nanoparticle. In one embodiment, the size of the gold nanoparticle is from about 3 nm to about 5 nm. In another embodiment, the size of the gold nanoparticle is about 4 nm.

In one embodiment, the metal nanoparticle exhibits super-paramagnetic behaviour at room temperature. In another embodiment, the metal nanoparticle exhibits ferrimagnetic behaviour at room temperature. In another embodiment, the metal nanoparticle exhibits ferromagnetic behaviour at room temperature.

A metal nanoparticle may comprise metals other than those indicated, provided that the metallic composition of the nanoparticle is not substantially altered. For example, in some embodiments an iron nanoparticle may comprise, in addition to iron, one or more additional metals, for example chromium, manganese, cobalt, nickel, copper, or zinc, as minor components. In one embodiment, the metal indicated comprises more than 70 mol % of the metal present in the metal nanoparticle. In another embodiment, the metal indicated comprises more than 75 mol % of the metal. In another embodiment, the metal indicated comprises more than 80 mol % of the metal. In another embodiment, the metal indicated comprises more than 85 mol % of the metal. In another embodiment, the metal indicated comprises more than 90 mol % of the metal. In another embodiment, the metal indicated comprises more than 95 mol % of the metal. In another embodiment, the metal indicated comprises more than 99 mol % of the metal.

The phosphopeptide of the metal nanoparticle-phosphopeptide complex is on the surface of the metal nanoparticle.

The phosphopeptide complex comprises two or more phosphorus-containing groups capable of interacting with the surface of the metal nanoparticle. In one embodiment, the phosphorus-containing groups interact with the surface of the metal nanoparticle. In one embodiment, the phosphorus-containing groups interact strongly with the surface of the metal nanoparticle. In one embodiment, the interaction is of sufficient strength to prevent dissociation of the phosphopeptide from the surface of the metal nanoparticle. In one embodiment, the phosphopeptide is adsorbed on the surface of the iron nanoparticle.

Without wishing to be bound by theory, the applicant believes that in some embodiments the phosphopeptide is adsorbed on the surface of the metal nanoparticle by the interaction between the phosphorus-containing groups and the surface of the metal nanoparticle.

The phosphopeptide comprises two or more contiguous peptide motifs. Each peptide motif is bound directly to another peptide motif via a peptide bond—i.e. the N-terminus of one peptide motif is bound to the C-terminus of another peptide motif. For example, when the phosphopeptide comprises three peptide motifs, the last amino acid of the first peptide motif is bound directly via a peptide bond to the first amino acid of the second peptide motif and the last amino acid of the second peptide motif is bound directly via a peptide bond to the first amino acid of the third peptide motif. The peptide backbone of each peptide motif in the phosphopeptide is therefore bound so as to form a contiguous sequence of amino acids.

Each phosphorus-containing group is bound to an amino acid in the two or more contiguous peptide motifs.

In one embodiment, the phosphorus-containing group comprises a phosphate, phosphonate, or pyrophosphate group. In one embodiment, the phosphorus-containing group comprises a phosphate or phosphonate group. In one embodiment, the phosphorus-containing group comprises a linker via which a phosphate or phosphonate group is bound to the amino acid.

In one embodiment, a phosphate or phosphonate group is disposed at the distal (with respect to the peptide backbone) end of the phosphorus-containing group. In one embodiment, the phosphorus-containing group is a phosphate or phosphonate group.

In one embodiment, the phosphopeptide comprises three or more phosphorus-containing groups. In another embodiment, the phosphopeptide comprises four or more phosphorus-containing groups.

In one embodiment, the phosphorus-containing groups are disposed on the two or more contiguous peptide motifs at regular intervals. In one embodiment, two or more phosphorus-containing groups are bound to amino acids at the equivalent position in each peptide motif. For example, if the first phosphorus-containing groups are bound to the first amino acid of the first peptide motif bearing a phosphorus-containing group, then the second and subsequent phosphorus groups will also be bound to the first amino acid of any peptide motifs bearing phosphorus-containing groups.

In one embodiment, each peptide motif contains one or more, two or more, or three or more phosphorus-containing groups. In one embodiment, each peptide motif contains one phosphorus-containing group. In one embodiment, each peptide motif contains two phosphorus-containing groups. In one embodiment, each peptide motif contains three phosphorus-containing groups.

In one embodiment, each peptide motif contains one phosphorus-containing group and the two or more phosphorus-containing groups are bound to amino acids at the equivalent position in each peptide motif.

Each peptide motif comprises a peptide backbone with the same number of amino acids. In one embodiment, each peptide motif is 3 or more amino acids in length. In another embodiment, each peptide motif is from 3 to 10 amino acids in length. In another embodiment, each peptide motif is from 3 to 7 amino acids in length. In another embodiment, each peptide motif is from 3 to 6 amino acids in length. In another embodiment, each peptide motif is 3, 4, or 5 amino acids in length. In another embodiment, each peptide motif is 3 amino acids in length.

In one embodiment, the phosphopeptide comprises 2 to 100 contiguous peptide motifs. In another embodiment, the phosphopeptide comprises 2 to 50 contiguous peptide motifs. In another embodiment, the phosphopeptide comprises 2 to 20 contiguous peptide motifs. In another embodiment, the phosphopeptide comprises 2 to 10 contiguous peptide motifs. In one embodiment, the phosphopeptide comprises from 2 to 4 contiguous peptide motifs.

The amino acids of each peptide motif may be natural or unnatural amino acids. As used herein an unnatural amino acid refers to any amino acid, modified amino acid, or amino acid analogue other than the following twenty genetically encoded α-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Unnatural amino acids include, but are not limited to, N-substituted α-amino acids; α,α-disubstituted amino acids, including cyclic quaternary amino acids; D-amino acids; β-amino acids, including β²- and β³-amino acids and β^{2,2}, β^{2,3}-, β³′3-disubstituted amino acids, including cyclic variants; 3- to 9-membered ring proline analogues; γ-amino acids; and homo amino acids. Examples of unnatural amino acids include D-glutamate, D-alanine, D-methyl-β-tyrosine, aminobutyric acid, γ-amino butyric acid, O-methyl-L-tyrosine, L-3-(2-naphthyl)alanine, o-methyl-phenylalanine, O-4-allyl-L-tyrosine, isopropyl-L-phenylalanine, p-iodo-phenylalanine, p-amino-L-phenylalanine, O-allyl-serine, allyl-L-glycine, α-methylalanine, N-propyl-glycine, N-propargyl-glycine, 3,3-dimethyl-proline, pipecolic acid, [2-(triazol-4-yl)methyl]glycine, [2-(triazol-1-yl)methyl]glycine, and the like. Further examples include 2-aminoadipic acid (Aad), 3-aminoadipic acid (bAad), beta-alanine (bAla), beta-aminopropionic acid (bAla), 2-aminobutyric acid (Abu), 4-aminobutyric acid (4Abu), piperidinic acid (4Abu), 6-aminocaproic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), desmosine (Des), 2,2′-diaminopimelic acid (Dpm), 2,3-diaminopropionic acid (Dpr), N-ethylglycine (EtGly), N-ethylasparagine (EtAsn), hydroxylysine (Hyl), allo-hydroxylysine (aHyl), 3-hydroxyproline (3Hyp), 4-hydroxyproline (4Hyp), isodesmosine (Ide), allo-isoleucine (aIle), N-methylglycine (MeGly), sarcosine (MeGly), N-methylisoleucine (MeIle), 6-N-methyllysine (MeLys), N-methylvaline (MeVal), norvaline (Nva), norleucine (Nle), ornithine (Orn), and the like.

Many unnatural amino acids are commercially available. Those that are not may be synthesised using standard methods known to those skilled in the art.

In one embodiment, each chiral amino acid of the peptide motifs is an L-amino acid. In one embodiment, the amino acids of the peptide motifs comprise D-amino acids. In another embodiment, each chiral amino acid of the peptide motif is a D-amino acid.

The amino acids at the equivalent position in each peptide motif have similar structural and/or electronic properties. The phosphopeptide therefore comprises a repeated sequence of amino acids having similar structural and/or electronic properties.

A person skilled in the art will appreciate that when a phosphorus-containing group is bound to an amino acid, the phosphorus-containing group is to be excluded from the consideration of whether the amino acid has structural and/or electronic properties similar to amino acids at the equivalent position in other peptide motifs. For example, a serine residue and an O-phospho-serine residue (i.e. a serine residue substituted with a phosphorus-containing group—the phospho group) meet the requirement of having similar structural and/or electronic properties.

In one embodiment, the amino acids at the equivalent position in each peptide motif have similar structural and electronic properties.

In one embodiment, the amino acids at the equivalent position in each peptide motif have similar hydrophobicity/hydrophilicity. In another embodiment, the amino acids at the equivalent position in each peptide motif have similar polarity.

Natural amino acids can be classified, for example, by hydrophobicity, hydrophilicity, and polarity. The same principles used to classify natural amino acids can be used to classify unnatural amino acids.

In one embodiment, the amino acids of each peptide motif are selected from one of the following categories: polar amino acids, non polar amino acids, hydrophobic amino acids, and non hydrophobic amino acids; and the amino acids at the equivalent position in each peptide motif are selected from the same category of amino acids.

Polar amino acids include, for example, aspartic acid, glutamic acid, arginine, histidine, lysine, serine, threonine, asparagine, glutamine, tyrosine, cysteine, and [2-(triazolyl)methyl]glycine. Non polar amino acids include, for example, glycine, proline, phenylalanine, tryptophan, valine, isoleucine, alanine, leucine, and methionine. Hydrophobic amino acids include, for example, phenylalanine, tryptophan, tyrosine, valine, isoleucine, alanine, leucine, and methionine. Non hydrophobic amino acids include, for example, glycine, proline, aspartic acid, glutamic acid, arginine, histidine, lysine, serine, threonine, asparagine, glutamine, and cysteine.

In one embodiment, the polar amino acid is a charged amino acid. Charged amino acids include, for example, arginine, histidine, lysine, aspartic acid, and glutamic acid.

In one embodiment, the charged amino acid is an acidic amino acid. Acidic amino acids include, for example, aspartic acid and glutamic acid. In one embodiment, the acidic amino acid is an aliphatic amino acid. Acidic aliphatic amino acids include, for example, aspartic acid and glutamic acid. In one embodiment, the acidic amino acid is a heteroaliphatic amino acid. In another embodiment, the acidic amino acid is an alicyclic amino acid. In another embodiment, the acidic amino acid is a heteroalicyclic amino acid. In another embodiment, the acidic amino acid is an aromatic amino acid. In another embodiment, the acidic amino acid is a heteroaromatic amino acid.

In another embodiment, the charged amino acid is a basic amino acid. Basic amino acids include, for example, arginine, histidine, and lysine. In one embodiment, the basic amino acid is an aliphatic amino acid. Basic aliphatic amino acids include, for example, arginine and lysine. In one embodiment, the basic amino acid is a heteroaliphatic amino acid. In another embodiment, the basic amino acid is an alicyclic amino acid. In another embodiment, the basic amino acid is a heteroalicyclic amino acid. In another embodiment, the basic amino acid is an aromatic amino acid. In another embodiment, the basic amino acid is a heteroaromatic amino acid. Basic heteroaromatic amino acids include, for example, histidine.

In another embodiment, the polar amino acid is a neutral polar amino acid. Neutral polar amino acids include, for example, serine, threonine, asparagine, glutamine, cysteine, and tyrosine. In one embodiment, the neutral polar amino acid is an aliphatic amino acid. Neutral polar aliphatic amino acids include, for example, serine, threonine, asparagine, glutamine, and cysteine. In one embodiment, the neutral polar amino acid is a heteroaliphatic amino acid. In another embodiment, the neutral polar amino acid is an alicyclic amino acid. In another embodiment, the neutral polar amino acid is a heteroalicyclic amino acid. In another embodiment, the neutral polar amino acid is an aromatic amino acid. Neutral polar amino acids include, for example, tyrosine. In another embodiment, the neutral polar amino acid is a heteroaromatic amino acid. Neutral polar heteroaromatic acids include, for example, [2-(triazolyl)methyl]glycine.

In another embodiment, the non polar amino acid is an aliphatic amino acid. Non polar aliphatic amino acids include, for example, glycine, valine, isoleucine, alanine, and leucine. In another embodiment, the non polar amino acid is an alicyclic amino acid. In another embodiment, the non polar amino acid is aromatic amino acid. Non polar aromatic amino acids include, for example, phenylalanine and tryptophan. In another embodiment, the non polar amino acid is a heteroaromatic amino acid. In another embodiment, the non polar amino acid is a heteroaliphatic amino acid. Non polar heteroaliphatic amino acids include, for example, methionine. In another embodiment, the non polar amino acid is a heteroalicyclic amino acid. Non polar heteroalicyclic amino acids include, for example, proline.

In another embodiment, the hydrophobic amino acid is an aliphatic amino acid. Hydrophobic aliphatic amino acids include, for example, valine, isoleucine, alanine, and leucine. In another embodiment, the hydrophobic amino acid is an alicyclic amino acid. In another embodiment, the hydrophobic amino acid is aromatic amino acid. Hydrophobic aromatic amino acids include, for example, phenylalanine, tryptamine, and tyrosine. In another embodiment, the hydrophobic amino acid is a heteroaromatic amino acid. In another embodiment, the hydrophobic amino acid is a heteroaliphatic amino acid. Hydrophobic heteroaliphatic amino acids include, for example, methionine. In another embodiment, the hydrophobic amino acid is a heteroalicyclic amino acid.

In another embodiment, the non-hydrophobic amino acid is a charged amino acid as described herein.

In another embodiment, the non hydrophobic amino acid is a neutral non hydrophobic amino acid. Neutral non hydrophobic aliphatic amino acids include, for example, serine, threonine, asparagine, glutamine, and cysteine. In another embodiment, the neutral non hydrophobic amino acid is an alicyclic amino acid. In another embodiment, the neutral non hydrophobic amino acid is aromatic amino acid. In another embodiment, the neutral non hydrophobic amino acid is a heteroaromatic amino acid. Neutral non hydrophobic heteroaromatic amino acids include, for example, [2-(triazolyl)methyl]glycine. In another embodiment, the neutral non hydrophobic amino acid is a heteroaliphatic amino acid. In another embodiment, the neutral non hydrophobic amino acid is a heteroalicyclic amino acid. Neutral non hydrophobic heteroalicyclic amino acids include, for example, proline.

In one embodiment, the amino acids at the equivalent position in each peptide motif are substantially identical. In one embodiment, the amino acids at the equivalent position in each peptide motif are identical.

Without wishing to be bound by theory, the applicant believes that the two or more contiguous peptide motifs, wherein the amino acids at the equivalent position in each peptide motif have similar structural and/or electronic properties may adopt a helical secondary structure in solution.

In one embodiment, the phosphopeptide favours a helical structure in solution. In one embodiment, the phosphopeptide has a helical structure in solution.

In one embodiment, the amino acid sequence of the two or more contiguous peptide motifs is such that the contiguous peptide motifs favour a helical structure in solution. In one embodiment, the amino acid sequence of the two or more contiguous peptide motifs is such that the contiguous peptide motifs favour an amphipathic helical structure in solution.

In one embodiment, the amino acid sequence of the phosphopeptide is such that the phosphopeptide favours a helical structure in solution. In one embodiment, the amino acid sequence of the phosphopeptide is such that the phosphopeptide favours an amphipathic helical structure in solution.

In one embodiment, the phosphorus-containing groups are presented on the same side of the helical structure.

In an amphipathic helix, non-polar and/or hydrophobic amino acids are predominantly on one side of the helix and polar and/or non hydrophobic amino acids are predominantly on the other, resulting in a peptide that is predominantly non-polar and/or hydrophobic on one face and polar and/or non hydrophobic on the other. Certain amino acids are known to favour the formation of a helical structure in solution, when incorporated into a peptide. Examples include alanine, valine, leucine, and phenylalanine. In one embodiment, each peptide motif comprises at least one amino acid that favours the formation of a helical structure in solution. Methods for determining the secondary structures of peptides in solution are known in the art, for example, circular dichromism spectroscopy.

In one embodiment, each peptide motif is a tripeptide. In one embodiment, a phosphorus-containing group is optionally bound to the first amino acid in each tripeptide motif. In another embodiment, a phosphorus-containing group is optionally bound to the second amino acid in each tripeptide motif. In another embodiment, a phosphorus-containing group is optionally bound to the third amino acid in each tripeptide motif.

In one embodiment, each peptide motif is a tripeptide, wherein: the first amino acid of each peptide motif at each instance is independently selected from one of the following categories: non polar amino acids and hydrophobic amino acids; the second amino acid of each peptide motif at each instance is independently selected from one of the following categories: non polar amino acids, hydrophobic amino acids, polar amino acids, and hydrophobic amino acids; the third amino acid of each peptide motif at each instance is independently selected from one of the following categories: polar amino acids and hydrophobic amino acids; a phosphorus-containing group capable of interacting with the surface of the metal nanoparticle is optionally bound to the third amino acid in each peptide motif; and the amino acids at the equivalent position in each peptide motif are selected from the same category of amino acids.

In one embodiment, each peptide motif is a tripeptide, wherein:
the first amino acid of each peptide motif at each instance is independently selected from one of the following categories: non polar aliphatic amino acids, non polar heteroalicyclic amino acids, non polar aromatic amino acids, hydrophobic aliphatic amino acids, and hydrophobic heteroalicyclic amino acids;
the second amino acid of each peptide motif at each instance is independently selected from one of the following categories: non polar aliphatic amino acids, non polar heteroalicyclic amino acids, non polar aromatic amino acids, non polar heteroaromatic amino acids, hydrophobic aliphatic amino acids, hydrophobic heteroalicyclic amino acids, hydrophobic aromatic amino acids, hydrophobic heteroaromatic amino acids, polar basic aliphatic amino acids, polar neutral aliphatic amino acids, polar basic heteroaromatic amino acids, non hydrophobic basic aliphatic amino acids, non hydrophobic neutral aliphatic amino acids, and non hydrophobic basic heteroaromatic amino acids;
third amino acid of each peptide motif at each instance is independently selected from one of the following categories: polar neutral aliphatic amino acids, polar neutral aromatic amino acids, polar neutral heteroaromatic amino acids, polar basic heteroaromatic amino acids, non hydrophobic neutral aliphatic amino acids, non hydrophobic neutral aromatic amino acids, non hydrophobic neutral heteroaromatic amino acids, and non hydrophobic basic heteroaromatic amino acids;
a phosphorus-containing group capable of interacting with the surface of the metal nanoparticle is optionally bound to the third amino acid in each peptide motif; and
the amino acids at the equivalent position in each peptide motif are selected from the same category of amino acids.

In one embodiment, each peptide motif is a tripeptide, wherein the first amino acid of each peptide motif at each instance is independently selected from one of the following categories: non polar amino acids and hydrophobic amino acids; the second amino acid of each peptide motif at each instance is independently selected from one of the following categories: non polar amino acids and hydrophobic amino acids; and the third amino acid of each peptide motif at each instance is independently selected from one of the following categories: polar amino acids and non hydrophobic amino acids; a phosphorus-containing group capable of interacting with the surface of the metal nanoparticle is optionally bound to the third amino acid in each peptide motif; and the amino acids at the equivalent position in each peptide motif are selected from the same category of amino acids.

In one embodiment, each peptide motif is a tripeptide, wherein
the first amino acid of each peptide motif at each instance is independently selected from one of the following categories: non polar aliphatic amino acids, non polar heteroalicyclic amino acids, non polar aromatic amino acids, hydrophobic aliphatic amino acids, and hydrophobic heteroalicyclic amino acids;
the second amino acid of each peptide motif at each instance is independently selected from one of the following categories: non polar aliphatic amino acids, non polar heteroalicyclic amino acids, non polar aromatic amino acids, hydrophobic aliphatic amino acids, and hydrophobic heteroalicyclic amino acids;
the third amino acid of each peptide motif at each instance is independently selected from one of the following categories: polar neutral aliphatic amino acids, polar neutral aromatic amino acids, non hydrophobic neutral aliphatic amino acids, and non hydrophobic neutral aromatic amino acids;
a phosphorus-containing group capable of interacting with the surface of the metal nanoparticle is optionally bound to the third amino acid in each peptide motif; and
the amino acids at the equivalent position in each peptide motif are selected from the same category of amino acids.

In one embodiment, each peptide motif is a tripeptide, wherein the first amino acid of each peptide motif at each instance is independently selected from the group consisting of alanine, isoleucine, leucine, valine, proline, phenylalanine, and glycine; the second amino acid of each peptide motif at each instance is independently selected from the group consisting of alanine, isoleucine, leucine, valine, proline, phenylalanine, and glycine; and the third amino acid of each peptide motif optionally bound to a phosphorus-containing group at each instance is independently selected from the group consisting of threonine, O-phospho-threonine, serine, O-phospho-serine, tyrosine, and O-phospho-tyrosine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, valine, and proline; the second amino acid of each peptide motif at each instance is independently selected from the group consisting of alanine, isoleucine, leucine, valine, and proline; and the third amino acid of each peptide motif optionally bound to a phosphorus-containing group at each instance is independently selected from the group consisting of threonine, O-phospho-threonine, serine, O-phospho-serine, tyrosine, and O-phospho-tyrosine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, and valine; the second amino acid of each peptide motif at each instance is independently selected from the group consisting of alanine, isoleucine, leucine, and valine; and the third amino acid of each peptide motif optionally bound to a phosphorus-containing group at each instance is independently selected from the group consisting of threonine, O-phospho-threonine, serine, O-phospho-serine, tyrosine, and O-phospho-tyrosine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is alanine; and the third amino acid of each peptide motif optionally bound to a phosphorus-containing group at each instance is independently selected from the group consisting of threonine or O-phospho-threonine; serine or O-phospho-serine; or tyrosine or O-phospho-tyrosine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from one of the following categories: non polar amino acids and hydrophobic amino acids; the second amino acid of each peptide motif at each instance is independently selected from one of the following categories: non polar amino acids, hydrophobic amino acids, polar amino acids, and hydrophobic amino acids; and the third amino acid of each peptide motif at each instance is independently selected from one of the following categories: polar amino acids and non hydrophobic amino acids; a phosphorus-containing group capable of interacting with the surface of the metal nanoparticle is optionally bound to the third amino acid in each peptide motif; and the amino acids at the equivalent position in each peptide motif are selected from the same category of amino acids.

In one embodiment, each peptide motif is a tripeptide, wherein
the first amino acid of each peptide motif at each instance is independently selected from one of the following categories: non polar aliphatic amino acids, non polar heteroalicyclic amino acids, non polar aromatic amino acids, hydrophobic aliphatic amino acids, and hydrophobic heteroalicyclic amino acids;
the second amino acid of each peptide motif at each instance is independently selected from one of the following categories: non polar aliphatic amino acids, non polar heteroalicyclic amino acids, non polar aromatic amino acids, non polar heteroaromatic amino acids, hydrophobic aliphatic amino acids, hydrophobic heteroalicyclic amino acids, hydrophobic aromatic amino acids, hydrophobic heteroaromatic amino acids, polar basic aliphatic amino acids, polar neutral aliphatic amino acids, polar basic heteroaromatic amino acids, non hydrophobic basic aliphatic amino acids, non hydrophobic neutral aliphatic amino acids, and non hydrophobic basic heteroaromatic amino acids;
the third amino acid of each peptide motif at each instance is independently selected from one of the following categories: polar neutral aromatic amino acids, polar neutral heteroaromatic amino acids, polar basic heteroaromatic amino acids, non hydrophobic neutral aromatic amino acids, non hydrophobic neutral heteroaromatic amino acids, and non hydrophobic basic heteroaromatic amino acids;
a phosphorus-containing group capable of interacting with the surface of the metal nanoparticle is optionally bound to the third amino acid in each peptide motif; and
the amino acids at the equivalent position in each peptide motif are selected from the same category of amino acids.

In one embodiment, each peptide motif is a tripeptide, wherein the first amino acid of each peptide motif at each instance is independently selected from the group consisting of alanine, isoleucine, leucine, valine, proline, phenylalanine, and glycine; the second amino acid of each peptide motif at each instance is independently selected from the group consisting of lysine, arginine, histidine, alanine, isoleucine, leucine, valine, proline, phenylalanine, tryptamine, and glycine; and the third amino acid of each peptide motif optionally bound to a phosphorus-containing group at each instance is independently selected from the group consisting of tyrosine, O-phospho-tyrosine, histidine, phospho-histidine, [2-(triazolyl)-C₁₋₆alkyl]glycine, and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, valine, and proline; the second amino acid of each peptide motif at each instance is independently selected from the group consisting of lysine and arginine; and the third amino acid of each peptide motif optionally bound to a phosphorus-containing group at each instance is independently selected from the group consisting of tyrosine, O-phospho-tyrosine, histidine, phospho-histidine, [2-(triazolyl)-C₁₋₆alkyl]glycine, and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, valine, and proline; the second amino acid of each peptide motif at each instance is independently selected from the group consisting of alanine, isoleucine, leucine, valine, and proline; and the third amino acid of each peptide motif optionally bound to a phosphorus-containing group at each instance is independently selected from the group consisting of tyrosine, O-phospho-tyrosine, histidine, phospho-histidine, [2-(triazolyl)-C₁₋₆alkyl]glycine, and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, and valine; the second amino acid of each peptide motif at each instance is independently selected from the group consisting of lysine and arginine; and the third amino acid of each peptide motif optionally bound to a phosphorus-containing group at each instance is independently selected from the group consisting of histidine, phospho-histidine, [2-(triazolyl)-C₁₋₆alkyl]glycine, and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif at each instance is independently selected from the group consisting of alanine, isoleucine, leucine, and valine; the second amino acid of each peptide motif at each instance is independently selected from the group consisting of alanine, isoleucine, leucine, and valine; and the third amino acid of each peptide motif optionally bound to a phosphorus-containing group at each instance is independently selected from the group consisting of histidine, phospho-histidine, [2-(triazolyl)-C₁₋₆alkyl]glycine, and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is alanine; and the third amino acid of each peptide motif optionally bound to a phosphorus-containing group at each instance is independently selected from the group consisting of threonine and O-phospho-threonine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is alanine; and the third amino acid of each peptide motif optionally bound to a phosphorus-containing group at each instance is independently selected from the group consisting of serine and O-phospho-serine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is alanine; and the third amino acid of each peptide motif optionally bound to a phosphorus-containing group at each instance is independently selected from the group consisting of tyrosine and O-phospho-tyrosine. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is lysine; and the third amino acid of each peptide motif optionally bound to a phosphorus-containing group is [2-(triazolyl)-C₁₋₆alkyl]glycine or [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, each peptide motif is a tripeptide, wherein the first amino acid of each peptide motif is alanine; the second amino acid of each peptide motif is alanine; and the third amino acid of each peptide motif optionally bound to a phosphorus-containing group is [2-(triazolyl)-C₁₋₆alkyl]glycine or and [2-(triazolyl)-C₁₋₆alkyl]glycine wherein the triazolyl ring is substituted with C₁₋₆alkylphosphonate. In one embodiment, the phosphopeptide comprises from 2 to 50 contiguous peptide motifs. In one embodiment, the phosphopeptide comprises from 2 to 20 contiguous peptide motifs.

In one embodiment, the [2-(triazolyl)-C₁₋₆alkyl]glycine is selected from the group consisting of [2-(triazolyl)-methyl]glycine, [2-(2-[triazolyl]-ethyl)]glycine, and [2-(3-[triazolyl]-propyl)]glycine. In another embodiment, the [2-(triazolyl)-C₁₋₆alkyl]glycine is [2-(triazolyl)-methyl]glycine.

In another embodiment, the [2-(triazolyl)-C₁₋₆alkyl]glycine is selected from the group consisting of [2-(triazol-4-yl)-methyl]glycine, [2-(2-[triazol-4-yl]-ethyl)]glycine, and [2-(3-[triazol-4-yl]-propyl)]glycine. In another embodiment, the [2-(triazolyl)-C₁₋₆alkyl]glycine is [2-(triazol-4-yl)-methyl]glycine.

In another embodiment, the [2-(triazolyl)-C₁₋₆alkyl]glycine is selected from the group consisting of [2-(triazol-1-yl)-methyl]glycine, [2-(2-[triazol-1-yl]-ethyl)]glycine, and [2-(3-[triazol-1-yl]-propyl)]glycine. In another embodiment, the [2-(triazol-1-yl)-C₁₋₆alkyl]glycine is [2-(triazol-1-yl)-methyl]glycine.

In one embodiment, the triazolyl in the [2-(triazolyl)-C₁₋₆alkyl]glycine is substituted with a phosphorus-containing group. In one embodiment, the [2-(triazolyl)-C₁₋₆alkyl]glycine is [2-(triazol-4-yl)-C₁₋₆alkyl]glycine, wherein the 1-position of the triazolyl ring is substituted with a phosphorus containing group. In another embodiment, the [2-(triazolyl)-C₁₋₆alkyl]glycine is [2-(triazol-1-yl)-C₁₋₆alkyl]glycine, wherein the 4-position of the triazolyl ring is substituted with a phosphorus containing group.

In one embodiment, the phosphopeptide comprises one or more groups that mitigate aggregation of the metal nanoparticle-phosphopeptide complex with metal nanoparticles or other metal nanoparticle-phosphopeptide complexes.

In one embodiment, the phosphopeptide is optionally substituted with one or more groups that mitigate aggregation of the metal nanoparticle-phosphopeptide complex with metal nanoparticles or other metal nanoparticle-phosphopeptide complexes.

A person skilled in the art will appreciate that when a group that mitigates aggregation is bound to an amino acid, the group that mitigates aggregation is to be excluded from the consideration of whether the amino acid has structural and/or electronic properties similar to amino acids at the equivalent position in other peptide motifs. For example, a serine residue and an O-sulfate-serine residue (i.e. a serine residue substituted with a group that mitigates aggregation—the sulfate group) meet the requirement of having similar structural and/or electronic properties.

In one embodiment, the group that mitigates aggregation of the metal nanoparticle-phosphopeptide complex with metal nanoparticles or other metal nanoparticle-phosphopeptide complexes mitigates aggregation by electrostatic stabilisation. In another embodiment, the group that mitigates aggregation of the metal nanoparticle-phosphopeptide complex with metal nanoparticles or other metal nanoparticle-phosphopeptide complexes mitigates aggregation by steric stabilisation. In another embodiment, the group that mitigates of the metal nanoparticle-phosphopeptide complex with metal nanoparticles or other metal nanoparticle-phosphopeptide complexes mitigates aggregation by electrostatic stabilisation and steric stabilisation.

In one embodiment, the group that mitigates aggregation is a charged group or a polymer.

In one embodiment, the group that mitigates aggregation comprises a charged group. In one embodiment, the charged group is selected from the group consisting of sulfate, C₁₋₆alkylsulfate, C₂₋₆alkenylsulfate, C₂₋₆alkynylsulfate, sulfonate, C₁₋₆alkylsulfonate, C₂₋₆alkenylsulfonate, and C₂₋₆alkynylsulfonate. In one embodiment the group that mitigates aggregation is selected from the group consisting of sulfate, C₁₋₆alkylsulfate, sulfonate, and C₁₋₆alkylsulfonate.

In another embodiment, the group that mitigates aggregation is a polymer. In one embodiment, the polymer is a short polymer. In one embodiment, the polymer is a polymer selected from the group consisting of polyethylene glycol, polyoxyethylene, polyethylene oxide, polyols, polysaccharides, and any combination thereof. In another embodiment, the polymer is a charged polymer. In one embodiment, the charged polymer is selected from the group consisting of a charged peptide, poly(styrene sulfonate), a zwitterionic polymer, and any combination thereof. In one embodiment, the charged peptide is a sulfated peptide. In one embodiment, the polymer is a synthetic polymer.

In another embodiment, the group that mitigates aggregation is a peptide comprising one or more hydrophilic and/or polar amino acids. In one embodiment, the peptide comprises from 2 to 20, from 2 to 19, from 2 to 18, from 2 to 17, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, or from 2 to 3 amino acids. In one embodiment, the peptide is a tri-, tetra-, penta-, hexa-, hepta-, octa-, nona-, deca-, undeca-, or dodeca-peptide. In one embodiment, the one or more hydrophilic and/or polar amino acids are charged amino acids. In one embodiment, the charged amino acid is aspartic acid or glutamic acid. In one embodiment, the peptide comprises or is a polyaspartic acid or polyglutamic acid sequence. In one embodiment, the peptide is a hexa or deca-aspartic acid or glutamic acid tag. In one embodiment, the peptide is attached to the two or more contiguous peptide motifs via the C-terminus or N-terminus of the two or more contiguous peptide motifs. In one embodiment, the peptide is attached via the N-terminus of the two or more contiguous peptide motifs.

In one embodiment, the phosphopeptide further comprises one or more groups that favour the formation of and/or stabilises a helical and/or amphipathic secondary structure in solution. In one embodiment, the group that favours the formation of and/or stabilises a helical and/or amphipathic secondary structure in solution comprises a hydrogen bond donor or acceptor. In one embodiment, the group that favours the formation of and/or stabilises a helical and/or amphipathic secondary structure in solution comprises an N-acetyl galactosamine residue.

In one embodiment, the phosphopeptide comprises an amino acid sequence of the formula (I):

Xaa¹-Xaa²-Xaa³ₙ (I)
wherein:
Xaa¹, Xaa², Xaa³, and n are as defined in the embodiment recited above.

Xaa¹, Xaa², and Xaa³, respectively, at each instance of n, have similar structural and/or electronic properties.

A person skilled in the art will appreciate that when a phosphorus-containing group or a group that mitigates aggregation is bound to R², the optionally substituted ring formed when R¹ and R² are taken together with nitrogen atom and carbon atom to which they are attached, or the optionally substituted ring formed when R² and R³ are taken together with the carbon atom to which they are attached in a Xaa¹, Xaa², or Xaa³, the phosphorus-containing group or group that mitigates aggregation is to be excluded from the consideration of whether Xaa¹, Xaa², and Xaa³, respectively, at each instance of n, have structural and/or electronic properties. For example, if Xaa¹ is a serine residue when n is 1 and an O-phospho-serine residue when n is 2 (i.e. a serine residue substituted with a phosphorus-containing group—the phospho group), these two residues are considered to meet the requirement of having similar structural and/or electronic properties.

In one embodiment, Xaa¹, Xaa², and Xaa³ respectively, at each instance of n have similar structural and electronic properties.

In one embodiment, n is an integer from 2 to 50. In one embodiment, n is an integer from 2 to 20. In another embodiment, n is an integer from 2 to 10. In another embodiment, n is an integer from 2 to 4.

In one embodiment, the phosphorus-containing group is selected from the group consisting of phosphate, C₁₋₆alkylphosphate, C₂₋₆alkenylphosphate, C₂₋₆alkynylphosphate, arylphosphate, C₁₋₆alkylarylphosphate, C₂₋₆alkenylarylphosphate, C₂₋₆alkynylarylphosphate, phosphonate, C₁₋₆alkylphosphonate, C₂₋₆alkenylphosphonate, C₂₋₆alkynylphosphonate, arylphosphonate, C₁₋₆alkylarylphosphonate, C₂₋₆alkenylarylphosphonate, C₂₋₆alkynylarylphosphonate. In one embodiment, the phosphorus-containing group is selected from the group consisting of phosphate, phosphonate, C₁₋₆alkylphosphate, and C₁₋₆alkylphosphonate.

The group that mitigates aggregation of the metal nanoparticle-phosphopeptide complex with metal nanoparticles or other metal nanoparticle-phosphopeptide complexes is as defined in any of the embodiments described above.

In one embodiment, Xaa¹ and Xaa² are each independently an amino acid residue of the formula (II) wherein:
R¹ is selected from the group consisting of hydrogen, C₁₋₆alkyl, C₂₋₆alkenyl, and C₂₋₆alkynyl, each of which is optionally substituted with one or more halo;
R² is selected from the group consisting of hydrogen, C₁₋₆alkyl, C₂₋₆alkenyl, C₂₋₆alkynyl, and C₁₋₆alkylaryl, each of which is optionally substituted with one or more substituents independently selected from the group consisting of C₁₋₆alkyl, C₂₋₆alkenyl, C₂₋₆alkynyl, C₁₋₆alkyoxy, C₁₋₆alkylthio, halo, C₁₋₆haloalkyl, and C₁₋₆haloalkoxy;
R³ is selected from the group consisting of hydrogen, C₁₋₆alkyl, C₂₋₆alkenyl, and C₂₋₆alkynyl, each of which is optionally substituted with one or more halo;
or R¹ and R² together with nitrogen atom and carbon atom to which they are attached form a 5- or 6-membered heterocyclyl ring optionally substituted with one or more substituents independently selected from the group consisting of C₁₋₆alkyl, C₂₋₆alkenyl, C₂₋₆alkynyl, C₁₋₆alkyoxy, C₁₋₆alkylthio, halo, C₁₋₆haloalkyl, and C₁₋₆haloalkoxy;
or R² and R³ together with the carbon atom to which they are attached form a 5- or 6-membered cycloalkyl or cycloalkenyl optionally substituted with one or more substituents independently selected from the group consisting of C₁₋₆alkyl, C₂₋₆alkenyl, C₂₋₆alkynyl, C₁₋₆alkyoxy, C₁₋₆alkylthio, halo, C₁₋₆haloalkyl, and C₁₋₆haloalkoxy; and
m is 0 or 1 and p is 0, or m is 0 and p is 0 or 1;
   or Xaa² at each instance of n is an amino acid residue of the formula (II) wherein:
R¹ is selected from the group consisting of hydrogen, C₁₋₆alkyl, C₂₋₆alkenyl, and C₂₋₆alkynyl, each of which is optionally substituted with one or more halo;
R² is selected from the group consisting of C₁₋₆alkyl, C₂₋₆alkenyl, C₂₋₆alkynyl, C₁₋₆alkylaryl, and C₁₋₆alkylheteroaryl, wherein each C₁₋₆alkyl, C₂₋₆alkenyl, and C₂₋₆alkynyl is substituted with hydroxyl, thiol, amino, amido, carboxyl, or guanidino, and optionally substituted with one or more substituents independently selected from the group consisting of hydroxyl, C₁₋₆alkyoxy, thiol, C₁₋₆alkylthio, halo, C₁₋₆haloalkyl, C₁₋₆haloalkoxy, cyano, and nitro, each C₁₋₆alkylaryl is substituted with hydroxyl, thiol, or amino, and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro, and each C₁₋₆alkylheteroaryl is optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro;
R³ is selected from the group consisting of hydrogen, C₁₋₆alkyl, C₂₋₆alkenyl, and C₂₋₆alkynyl, each of which is optionally substituted with one or more halo;
or R¹ and R² together with nitrogen atom and carbon atom to which they are attached form a 5- or 6-membered heterocyclyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, cyano, and nitro;
or R² and R³ together with the carbon atom to which they are attached form a 5- or 6-membered cycloalkyl or cycloalkenyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, or a 5- or 6-membered heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy; and
m is 0 or 1 and p is 0, or m is 0 and p is 0 or 1; and

Xaa³ at each instance of n is an amino acid residue of the formula (II) wherein:
R¹ is selected from the group consisting of hydrogen, C₁₋₆alkyl, C₂₋₆alkenyl, and C₂₋₆alkynyl, each of which is optionally substituted with one or more halo;
R² is selected from the group consisting of C₁₋₆alkyl, C₂₋₆alkenyl, C₂₋₆alkynyl, C₁₋₆alkylaryl, and C₁₋₆alkylheteroaryl, wherein each C₁₋₆alkyl, C₂₋₆alkenyl, and C₂₋₆alkynyl is substituted with hydroxyl, thiol, amino, amido, carboxyl, or guanidino, and optionally substituted with one or more substituents independently selected from the group consisting of hydroxyl, C₁₋₆alkyoxy, thiol, C₁₋₆alkylthio, halo, C₁₋₆haloalkyl, C₁₋₆haloalkoxy, cyano, and nitro, each C₁₋₆alkylaryl is substituted with hydroxyl, thiol, or amino, and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro, and each C₁₋₆alkylheteroaryl is optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, amino, cyano, and nitro;
R³ is selected from the group consisting of hydrogen, C₁₋₆alkyl, C₂₋₆alkenyl, and C₂₋₆alkynyl, each of which is optionally substituted with one or more halo;
or R¹ and R² together with nitrogen atom and carbon atom to which they are attached form a 5- or 6-membered heterocyclyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, cyano, and nitro;
or R² and R³ together with the carbon atom to which they are attached form a 5- or 6-membered cycloalkyl or cycloalkenyl ring substituted with hydroxyl or thiol and optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy, or a 5- or 6-membered heterocyclyl ring optionally substituted with one or more substituents independently selected from the group consisting of C₁₋₆alkyl, C₂₋₆alkenyl, C₂₋₆alkynyl, hydroxyl, C₁₋₆alkyoxy, thiol, C₁₋₆alkylthio, halo, C₁₋₆haloalkyl, C₁₋₆haloalkoxy; and
m is 0 or 1 and p is 0, or m is 0 and p is 0 or 1; and
   a phosphorus-containing group is optionally bound to R², the optionally substituted ring formed when R¹ and R² are taken together with nitrogen atom and carbon atom to which they are attached, or the optionally substituted ring formed when R² and R³ are taken together with the carbon atom to which they are attached in Xaa³;
wherein the phosphorus-containing group is a phosphate, phosphonate, C₁₋₆alkylphosphate, or C₁₋₆alkylphosphonate.

In another embodiment, Xaa¹ and Xaa² are each independently an amino acid residue of the formula (II) wherein:
R¹ and R³ are each hydrogen;
R² is selected from the group consisting of hydrogen, C₁₋₆alkyl and C₁₋₆alkylaryl, wherein each C₁₋₆alkyl is optionally substituted with C₁₋₆alkylthio;
or R¹ and R² together with nitrogen atom and carbon atom to which they are attached form a pyrrolidinyl ring; and
m is 0 and p is 0;
   or Xaa² at each instance of n is an amino acid residue of the formula (II) wherein:
R¹ and R³ are each hydrogen;
R² is selected from the group consisting of C₁₋₆alkyl, C₁₋₆alkylaryl, and C₁₋₆alkylheteroaryl, wherein each C₁₋₆alkyl is substituted with hydroxyl, thiol, amino, amido, carboxyl, or guanidino, and each C₁₋₆alkylaryl is substituted with hydroxyl; and
m is 0 and p is 0; and

Xaa³ at each instance of n is an amino acid residue of the formula (II) wherein:
R¹ and R³ are each hydrogen;
R² is selected from the group consisting of C₁₋₆alkyl, C₁₋₆alkylaryl, and C₁₋₆alkylheteroaryl, wherein each C₁₋₆alkyl is substituted with hydroxyl, thiol, amino, amido, carboxyl, or guanidino, and each C₁₋₆alkylaryl is substituted with hydroxyl;
R² is optionally substituted with phosphate, phosphonate, C₁₋₆alkylphosphate, or C₁₋₆alkylphosphonate; and
m is 0 and p is 0; and
   a phosphorus-containing group is optionally bound to R² in Xaa³;
wherein the phosphorus-containing group is a phosphate, phosphonate, C₁₋₆alkylphosphate, or C₁₋₆alkylphosphonate.

In one embodiment, the phosphopeptide comprises an amino acid sequence of the formula (III):

Xaa¹-Xaa²-Xaa³-Xaa⁴ₙ (III)
wherein:
Xaa¹, Xaa², Xaa³, and n are as defined in any of the embodiments recited above; and
Xaa⁴ is an amino acid residue of the formula (II) as defined in any of the embodiments described above.

In another embodiment, the phosphopeptide comprises an amino acid sequence of the formula (IV):

Xaa¹-Xaa²-Xaa³-Xaa⁴-Xaa⁵ₙ (IV)
wherein:
Xaa¹, Xaa², Xaa³, and n are as defined in any of the embodiments recited above; and
Xaa⁴ and Xaa⁵ are each independently an amino acid residue of the formula (II) as defined in any of the embodiments described above.

In another embodiment, the phosphopeptide comprises an amino acid sequence of the formula (V):

Xaa¹-Xaa²-Xaa³-Xaa⁴-Xaa⁵-Xaa⁶ₙ (V)
wherein:
Xaa¹, Xaa², Xaa³, and n are as defined in any of the embodiments recited above; and
Xaa⁴, Xaa⁵, and Xaa⁶ are each independently an amino acid residue of the formula (II) as defined in any of the embodiments described above.

The N-terminus and C-terminus of the amino acid sequences of the formulae (I), (III), (IV), and (V) may be bound to any suitable substituent, provided that the substituent does not adversely affect the ability of the phosphopeptide to interact with the metal nanoparticle.

In one embodiment, a group that mitigates aggregation of the metal nanoparticles as defined in any of the embodiments described herein is attached to the N-terminus or C-terminus of the amino acid sequence. In one embodiment, the group that mitigates aggregation is a charged peptide. In one embodiment, the charged peptide comprises a polyaspartic acid or polyglutamic acid sequence.

In one embodiment, the phosphopeptide is a compound of the formula (VI):

A¹Xaa¹-Xaa²-Xaa³ₙA² (IV)
wherein:
A¹ is selected from the group consisting of hydrogen, an amino acid, a peptide, and group that mitigates aggregation of the metal nanoparticle-phosphopeptide complex with metal nanoparticles or other metal nanoparticle-phosphopeptide complexes;
A² is selected from the group consisting of hydroxyl, an amino acid, a peptide, and group that mitigates aggregation of the metal nanoparticle-phosphopeptide complex with metal nanoparticles or other metal nanoparticle-phosphopeptide complexes; and
Xaa¹, Xaa², and Xaa³ are as defined in any of the embodiments relating to the amino acid sequence of formula (I) described herein.

A¹ is bound to the N-terminus of the amino acid sequence and A² is bound to the C-terminus of the amino acid sequence.

In one embodiment, A¹ is selected from the group consisting of hydrogen, an amino acid, and a peptide; and A² is selected from the group consisting of hydroxyl, an amino acid, and a peptide.

In one embodiment, the peptide comprises from 2 to 100 amino acid residues. In another embodiment, the peptide comprises from 2 to 75 amino acid residues. In another embodiment, the peptide comprises from 2 to 50 amino acid residues. In another embodiment the peptide comprises from 2 to 20 amino acid residues. In another embodiment, the peptide comprises from 2 to 10 amino acid residues. In another embodiment, the peptide comprises from 2 to 6 amino acid residues.

In one embodiment, the peptide mitigates aggregation of the metal nanoparticles. In one embodiment, the peptide comprises one or more hydrophilic and/or polar amino acids. In one embodiment, the peptide comprises from 2 to 20, from 2 to 19, from 2 to 18, from 2 to 17, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, or from 2 to 3 amino acid residues. In one embodiment, the peptide is a tri-, tetra-, penta-, hexa-, hepta-, octa-, nona-, deca-, undeca-, or dodeca-peptide. In one embodiment, the hydrophilic and/or polar amino acids are charged amino acids. In one embodiment, the charged amino acid is aspartic acid or glutamic acid. In one embodiment, the peptide comprises or is a polyaspartic acid or polyglutamic acid sequence. In one embodiment, the peptide is a hexa- or deca-aspartic acid or glutamic acid tag.

In one embodiment, A¹ is a fatty acid ester. In another embodiment, A¹ is dodecanoyl.

In another embodiment, A¹ is peptide. In one embodiment, the peptide mitigates aggregation of the metal nanoparticles. In one embodiment, the peptide is a charged peptide. In one embodiment, the charged peptide comprises one or more aspartic acid or glutamic acid residues. In one embodiment, the peptide comprises or is a polyaspartic acid or polyglutamic acid tag. In one embodiment, the peptide is DDDDDD-, wherein each D represents an aspartic acid residue. In another embodiment, the peptide is EEEEEE- or EEEEEEEEEE-, wherein each E represents a glutamic acid residue.

In one embodiment, the phosphopeptide is a compound of the formula (VII):

A¹Xaa¹-Xaa²-Xaa³-Xaa⁴ₙA² (VII)
wherein:
Xaa¹, Xaa², Xaa³, Xaa⁴, n, A¹, and A² are as defined in any of the embodiments described above.

In another embodiment, the phosphopeptide comprises an amino acid sequence of the formula (IV):

A¹Xaa¹-Xaa²-Xaa³-Xaa⁴-Xaa⁵ₙA² (IV)
wherein:
Xaa¹, Xaa², Xaa³, Xaa⁴, Xaa⁵, n, A¹, and A² are as defined in any of the embodiments described above.

In another embodiment, the phosphopeptide comprises an amino acid sequence of the formula (V):

A¹Xaa¹-Xaa²-Xaa³-Xaa⁴-Xaa⁵-Xaa⁶ₙA² (V)
wherein:
Xaa¹, Xaa², Xaa³, Xaa⁴, Xaa⁵, Xaa⁶, n, A¹, and A² are as defined in any of the embodiments described above.

In one embodiment, the phosphopeptide is selected from the group consisting of:

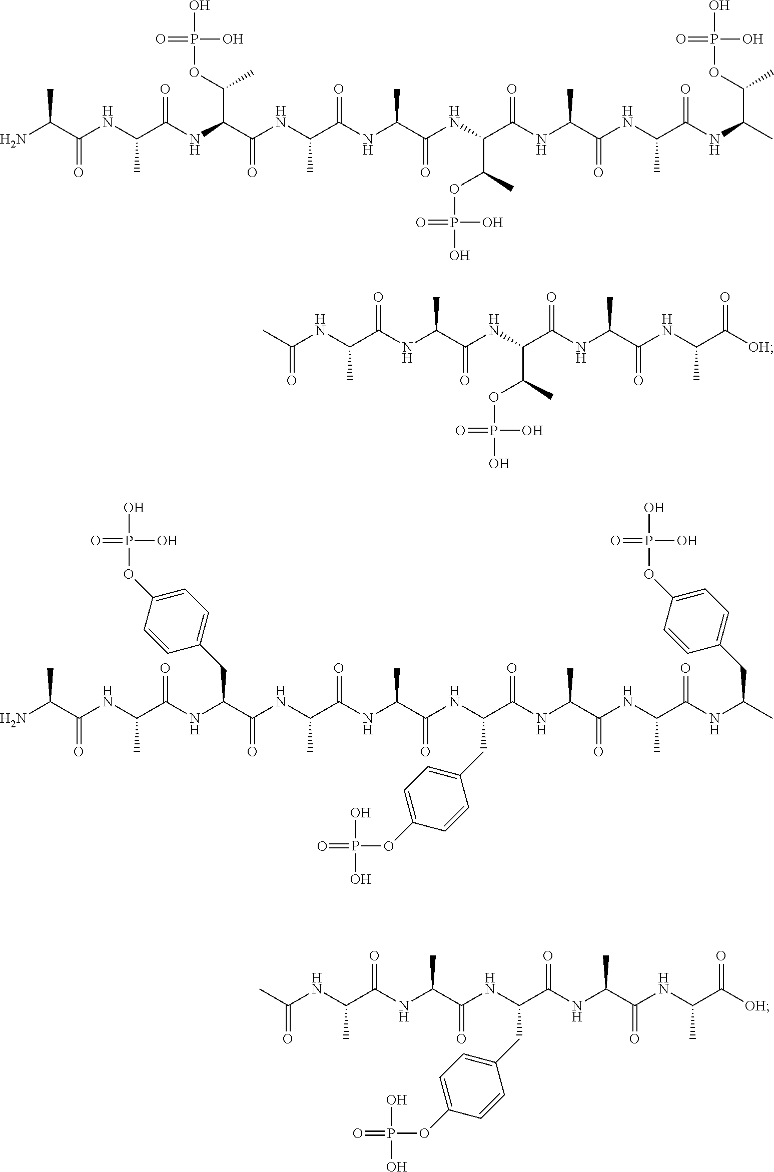

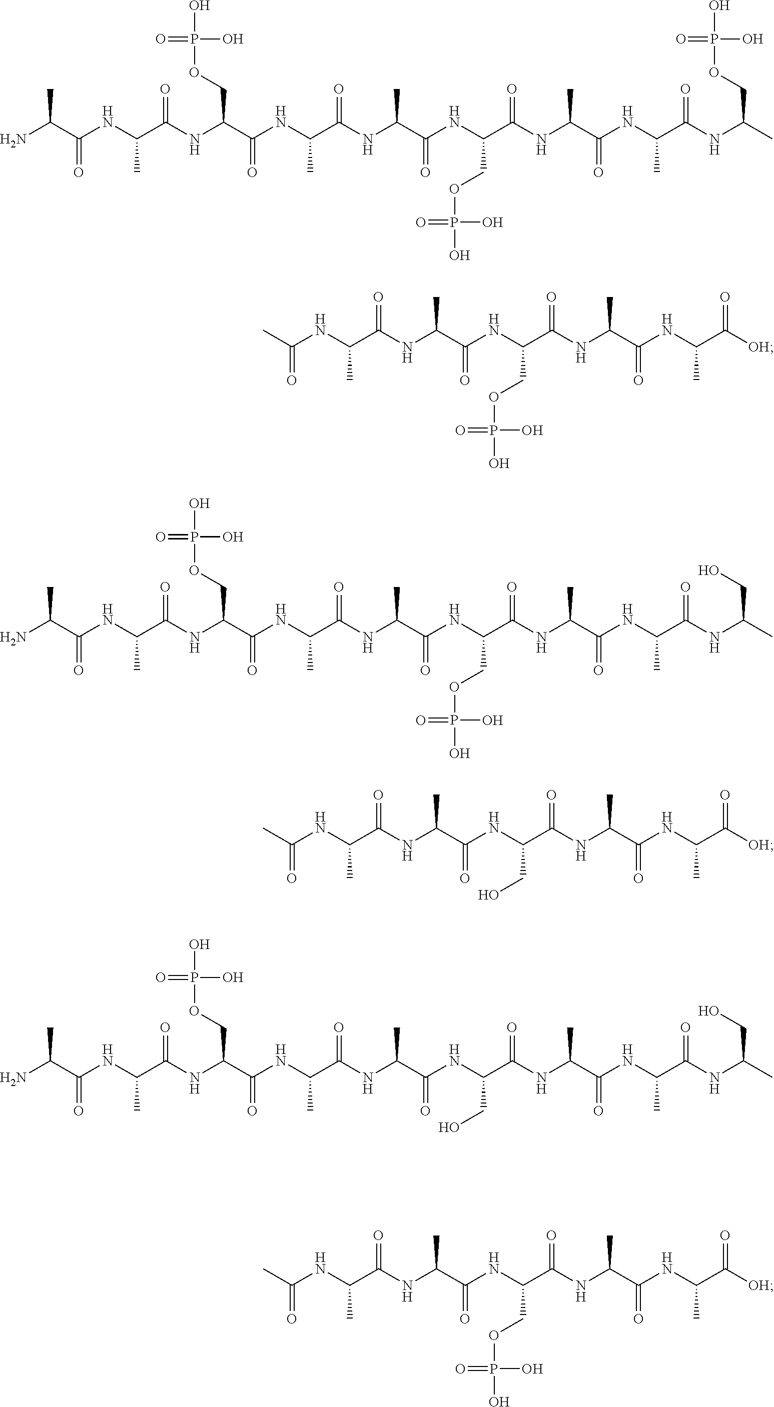

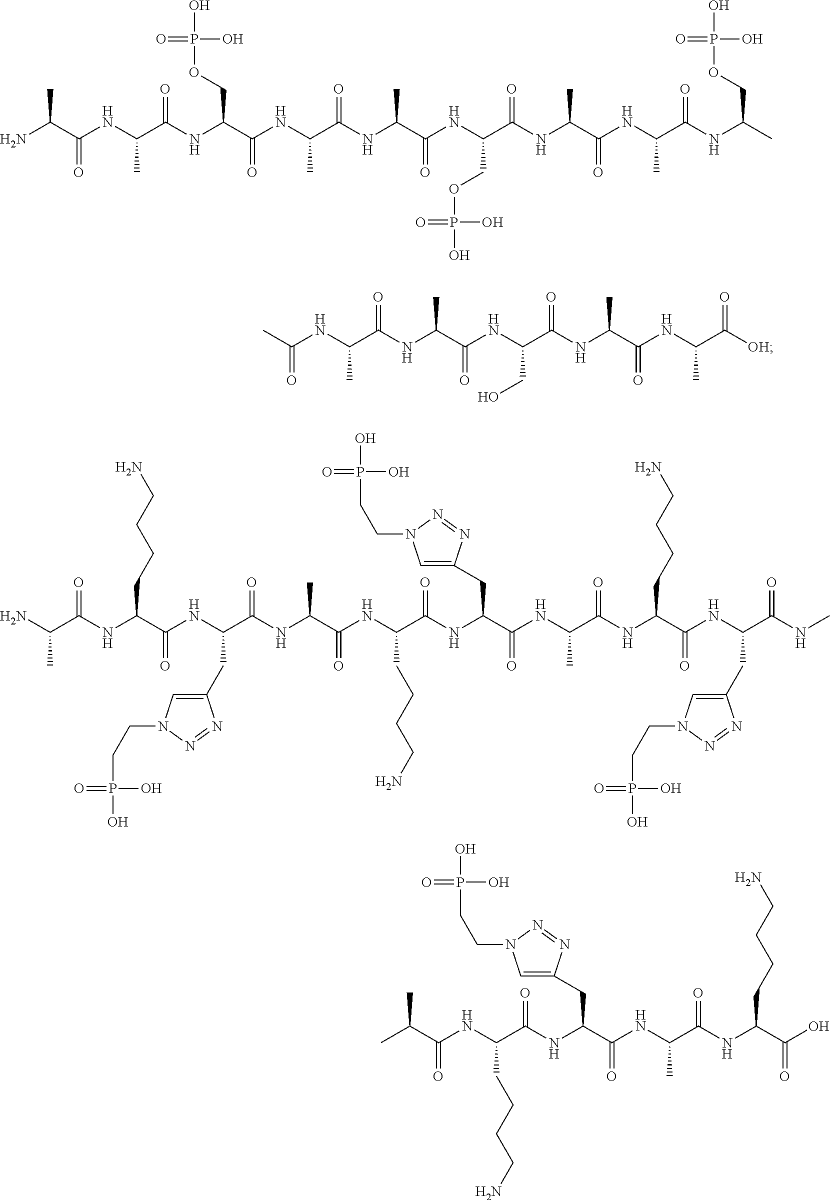

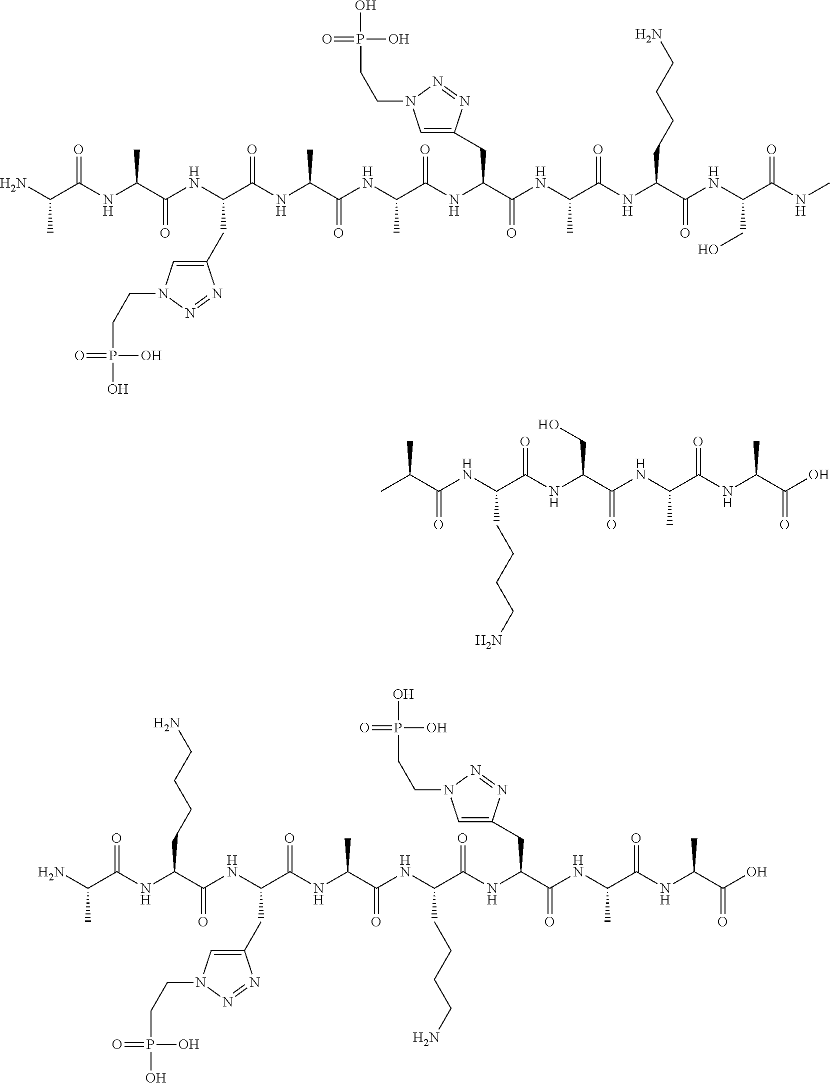

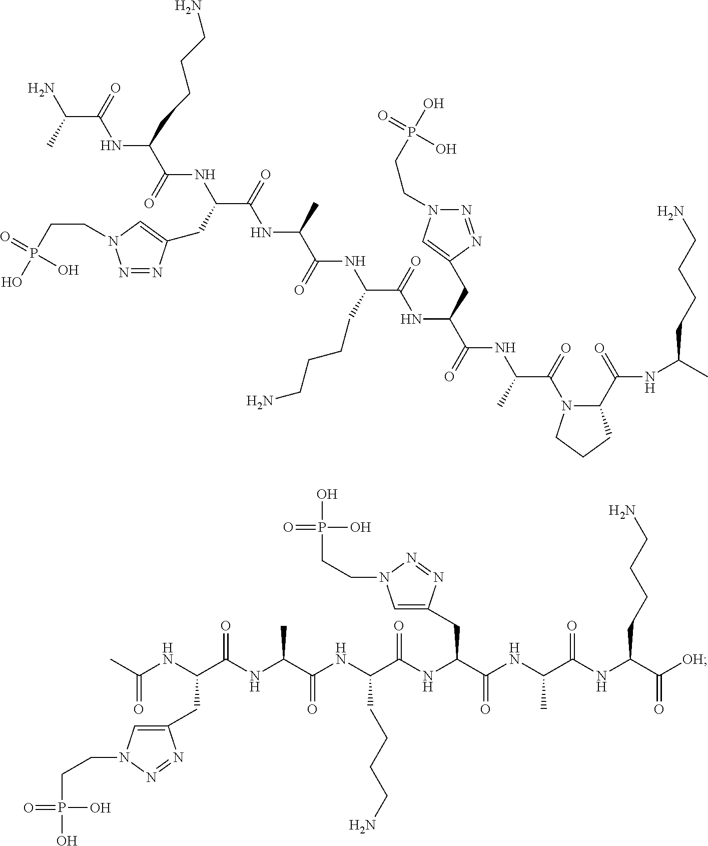 [SMILES: C[C@H](CCCCN)NC(=O)[C@@H]1CCCN1C(=O)[C@H](C)NC(=O)[C@H](Cc1cn(CCP(=O)(O)O)nn1)NC(=O)[C@H](CCCCN)NC(=O)[C@H](C)NC(=O)[C@H](Cc1cn(CCP(=O)(O)O)nn1)NC(=O)[C@H](CCCCN)NC(=O)[C@H](C)N ; CC(=O)N[C@@H](Cc1cn(CCP(=O)(O)O)nn1)C(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)O]

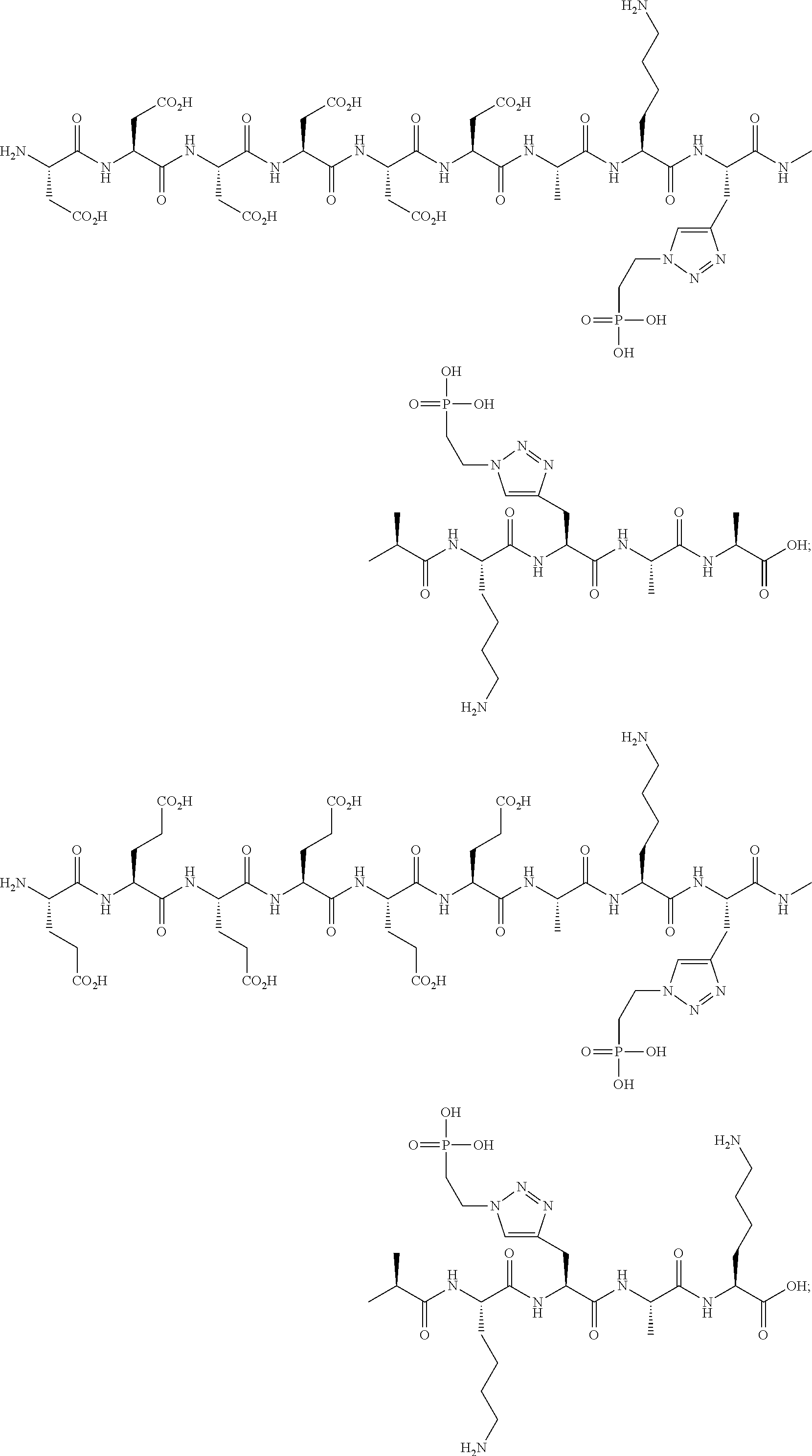

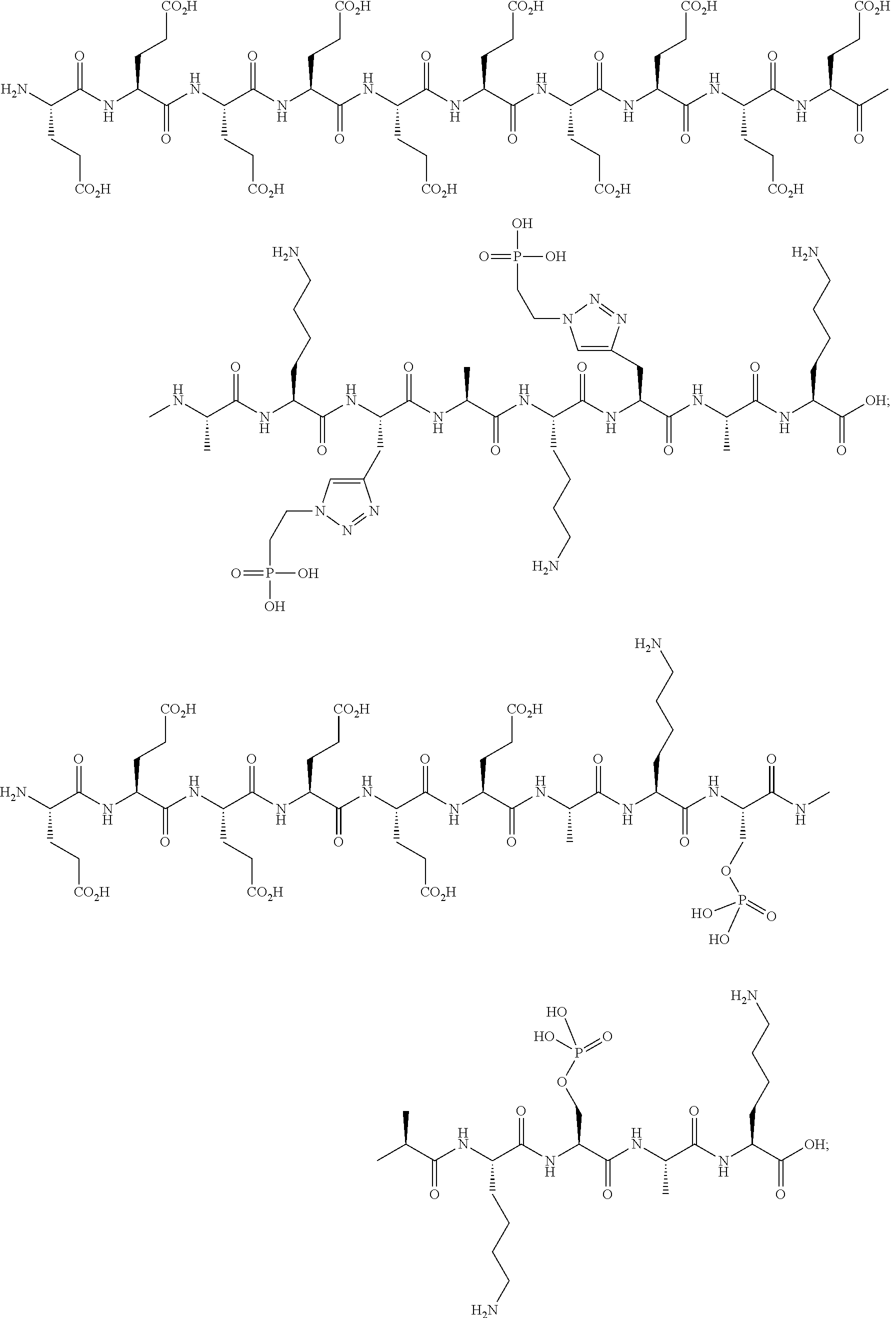

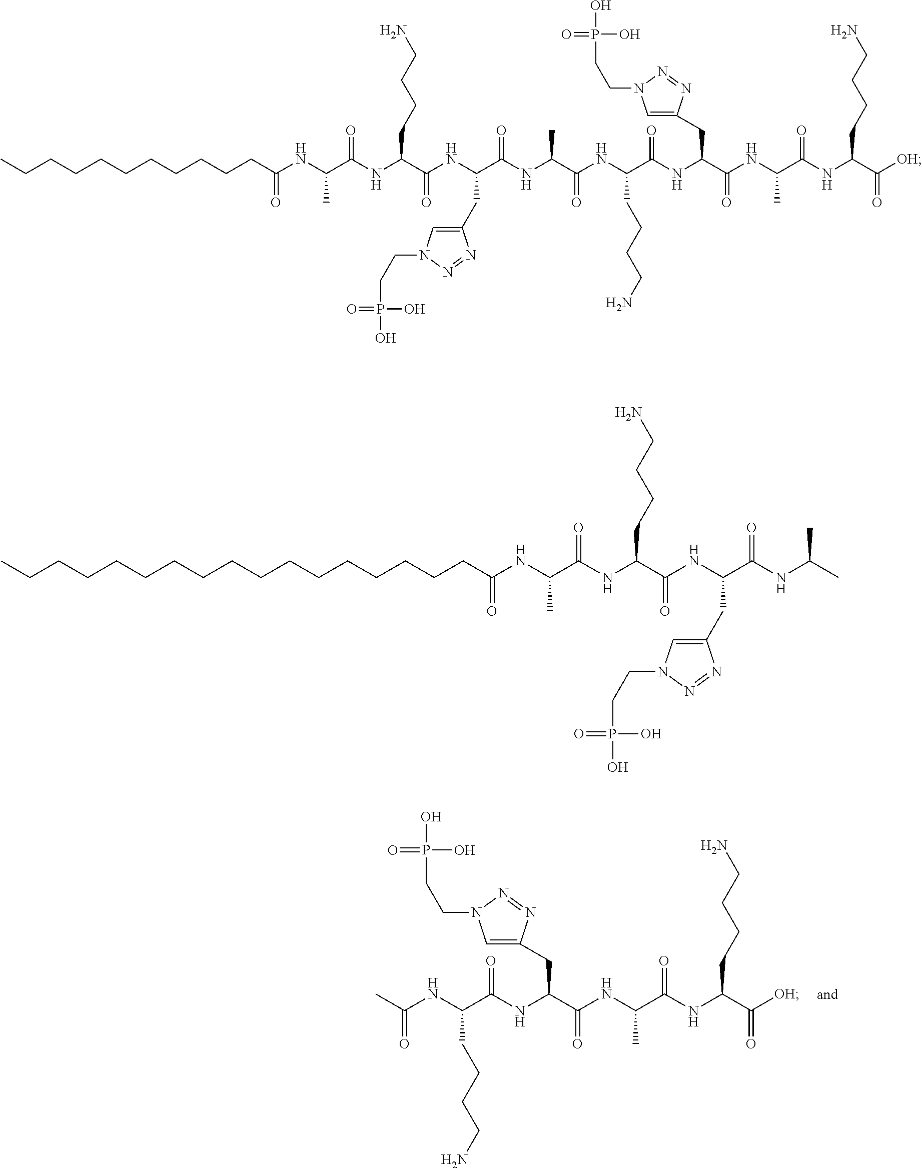

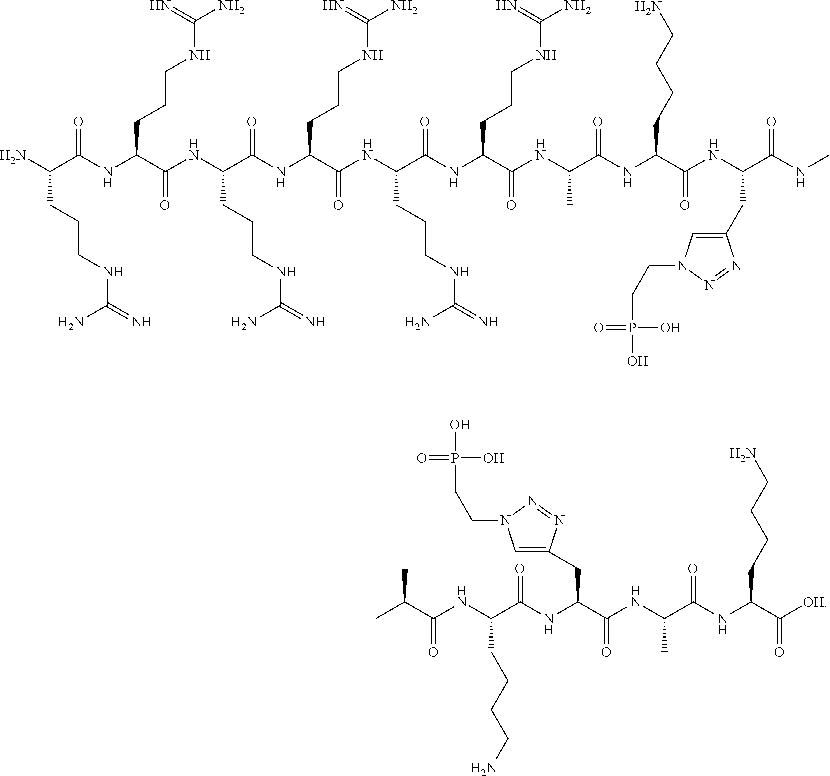

Without wishing to be bound by theory, the applicant believes that the phosphopeptides of the present invention form spherical cavities in which metal nanoparticles are located. The phosphorus containing groups capable of interacting with the surface of the metal nanoparticle are oriented towards the center of the cavity. The side chains of the other amino acid residues may be oriented away from the center of the cavity. The side chains orientated away from the centre of the cavity may mitigate aggregation, if they are of an appropriate nature (e.g. hydrophobic, hydrophilic, etc.) having regard to the liquid reaction medium (e.g. its polarity, pH, etc.).

In one embodiment, each peptide motif comprises at least one amino acid that mitigates aggregation of metal nanoparticles or other metal nanoparticle-phosphopeptide complexes in the liquid reaction medium.

Without wishing to be bound by theory, the applicant also believes that the position of the phosphorus containing groups in the phosphopeptide can control the size and shape of the metal nanoparticle.

In one embodiment, the iron nanoparticle-phosphopeptide complex comprises more than one type of phosphopeptide—i.e. phosphopeptides of different chemical structure.

In a further aspect, the present invention provides a phosphopeptide as defined in any of the embodiments described herein.

The phosphopeptide may be prepared by any suitable method known in the art. In one embodiment, amino acid sequence of the phosphopeptide is prepared by solid phase synthesis.

Solid-phase phase synthesis is commonly used for the preparation of peptides. Generally the procedure involves immobilising the first amino acid of the peptide on a solid support, usually via a linker. Examples of solid supports include Merrifield resin, ArgoGel® resin, Tentagel® resin, PEG-PS resin, CLEAR® resin, PEGA resin, and the like. A person skilled in the art will be able to select an appropriate solid support without undue experimentation. The next amino acid of the sequence, wherein the N-terminus of the amino acid is protected, is then coupled. If the N-terminus of the solid phase bound amino acid is protected, the protecting group will need to be removed prior to coupling. Examples of common protecting groups include Fmoc (9-fluorenylmethyloxycarbonyl) and Boc (tert-butyloxycarbonyl). Boc groups can be removed using acids, for example, trifluoroacetic acid. Fmoc groups can be removed using base, for example, piperidine. Examples of suitable solvents for the deprotection reaction include, but are not limited to, N,N-dimethylformamide, dimethylsulfoxide, dichloromethane, acetonitrile, and mixtures thereof.

The coupling reaction is typically carried out in the presence of one or more activating agents. Examples of activating agents include DCC, DIC, HBTU, HATU, PyBOP, BOP, and the like. An agent that reduces the racemisation, for example, HOBt or HOAt, can also be included. The coupling reaction is carried out in any suitable solvent. Examples of suitable solvent include, but are not limited to, N,N-dimethylformamide, dimethylsulfoxide, dichloromethane, acetonitrile, water, and mixtures thereof. The solid phase bound peptide is then washed to remove any residual reagents from the coupling reaction, and then subjected to a deprotection step.

The next amino acid of the sequence, wherein the N-terminus of the amino acid is protected, is then coupled. The sequence is repeated as necessary to prepare the desired peptide sequence. The peptide is then cleaved from the solid phase support. The crude peptide is typically purified. Purification is usually carried out by preparative HPLC.

Alternative procedures that employ more convergent strategies may involve coupling peptide fragments comprising several amino acids, rather than individual amino acids.

The phosphopeptides of the present invention comprise two or more phosphorus-containing groups capable of interacting with the surface of the metal nanoparticle. Each phosphorus-containing group is bound to an amino acid of the two or more contiguous peptide motifs. The phosphorus containing groups may be introduced into the peptide by any suitable method known in the art.

In one embodiment, the phosphorus-containing groups are introduced into the peptide by coupling an amino acid comprising the phosphorus-containing group to the peptide. For example, commercially available Fmoc-Ser(HPO₃Bn)-OH may be coupled with the peptide to introduce a serine amino acid bearing a phosphate group. Those of skill in the art will appreciate that the reaction conditions for subsequent deprotection and coupling reactions may need to be modified to account for the presence of the phosphorus-containing group in the peptide.

In another embodiment, the peptide may be reacted with a suitable precursor of the phosphorus-containing group.

In one embodiment, the phosphorus-containing groups are introduced using an azide-alkyne Huisgen cycloaddition ‘click’ reaction. A peptide comprising a α-propargyl glycine amino acid is reacted with 2-azidoethylphosphonic acid in the presence of a copper catalyst to from a 1,2,3-triazole ring substituted with the phosphorus-containing group (an ethylphosphonic acid group). Alternatively, a peptide comprising an azido-amino acid is reacted with a propargyl phosphonic acid.

In another embodiment, the phosphorus-containing groups are introduced using a nitrile-azide cycloaddition reaction. The nitrile-azide cycloaddition reaction provides a tetrazole ring substituted with the phosphorus containing group.

In a further aspect, the present invention provides a composition comprising a plurality of metal nanoparticles and a phosphopeptide of the present invention.

In a further aspect, the present invention provides a composition comprising a plurality of metal nanoparticle-phosphopeptide complexes of the present invention.

In one embodiment, the compositions further comprise a solvent in which the meal nanoparticle-phosphopeptide complexes are suspended. Advantageously, the metal nanoparticles and phosphopeptide of the present invention or metal nanoparticle-phosphopeptide complexes of the present invention form stable suspensions in suitable solvents, for example water. In one embodiment, the suspension is stable for at least one day.

In another embodiment, the compositions are in the form of a powder. The powder may be treated with a solvent to provide a suspension of the metal nanoparticles and phosphopeptide of the present invention or metal nanoparticle-phosphopeptide complexes. Advantageously, the metal nanoparticle-phosphopeptide complexes of the present invention may readily disperse when combined with suitable solvents, for example water, to provide stable suspensions. In one embodiment, the suspension is stable for at least one day. In one embodiment, the suspension is stable for at least 2, 4, 6, 8, 12, 18, or 24 h.

In one embodiment, the compositions comprise a pharmaceutically acceptable carrier, excipient, or diluent. Any suitable carrier, excipient, or diluent known in the pharmaceutical arts may used. In one embodiment the compositions are for use in the treatment of cancer. In another embodiment, the compositions are for use as a contrast agent for contrast enhancement in medical imaging.

In one embodiment, the compositions comprise more than one type of metal nanoparticle-phosphopeptide complex. In another embodiment, the compositions comprise more than one type of metal nanoparticles, for example, iron-iron oxide core-shell nanoparticles and iron oxide nanoparticles. In another embodiment, the compositions comprise more than one type of phosphopeptide.

In a further aspect, the present invention provides a method for preparing a metal nanoparticle-phosphopeptide complex, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs;
      in a liquid reaction medium under conditions that form a metal nanoparticle-phosphopeptide complex.

In a further aspect, the present invention provides a method for preparing metal nanoparticles, the method comprising contacting
a metal compound;
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
a reducing agent
   in a liquid reaction medium to form metal nanoparticles.

In a further aspect, the present invention provides a method for preparing a metal nanoparticle-phosphopeptide complex, the method comprising contacting
a metal compound;
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
a reducing agent
   in a liquid reaction medium to form a metal nanoparticle-phosphopeptide complex.
The methods advantageously provide a one-pot route for producing metal nanoparticles and metal nanoparticle-phosphopeptide complexes. Any suitable metal compound may be used.

The metal is reducible by a reducing agent in a liquid reaction medium (i.e. in solution). In one embodiment, the metal compound is reducible in a liquid reaction medium comprising water, an organic solvent, or a mixture thereof. In one embodiment, the metal compound is reducible in a liquid reaction medium comprising water. In another embodiment, the metal compound is reducible in an aqueous liquid reaction medium.

In one embodiment, the metal compound is at least partially soluble in the liquid reaction medium. In one embodiment, the metal compound is soluble.

The reaction is carried out under conditions conducive to formation of the nanoparticles.

In one embodiment, the metal compound is a metal salt. Examples of metal salts include but are not limited to metal chlorides, nitrates, citrates, oxalates, sulfates, acetates, and the like. Other suitable salts will be apparent to those skilled in the art.

In one embodiment, the metal compound is FeSO₄, Pt(NH₃)₄(NO₃)₂, Pt(NH₃)₄(OH)₂, H₂PtCl₄, PdCl₂, RuCl₃, Ag(CF₃COO), AgNO₃, IrCl₃, RhCl₃, AuCl₃, Cu(OAc)₂, CoCl₃, Ni(OAc)₂.

In one embodiment, the metal compound is an iron compound.

In one embodiment, the iron compound is an iron (II) or (III) compound. In another embodiment the iron compound is an iron (III) compound. In another embodiment, the iron compound is an iron (II) compound.

In one embodiment, the iron compound is an organo-iron compound. Examples of organo-iron compounds include ferrocene and iron pentacarbonyl.

In one embodiment, the iron compound is an iron salt. In one embodiment, the iron salt is selected from the group consisting of iron sulfates, iron acetoacetonates, iron oxalates, iron citrates, iron ammonium sulfates, iron sulfates, iron chlorides, and iron nitrates. In another embodiment, the iron salt is an iron (II) salt.

One embodiment utilises iron (II) sulfate.

The phosphopeptide affects the nucleation and growth of the metal nanoparticles in the liquid reaction medium. The phosphopeptide is as defined in any of the embodiments described herein.

In one embodiment, the molar concentration of phosphopeptide relative to metal is low. In one embodiment, the molar concentration of phosphopeptide relative to metal is less than about 25%. In another embodiment, the molar concentration of phosphopeptide relative to metal is less than about 15%. In one embodiment, the molar concentration of phosphopeptide relative to metal is about 5%.

Without wishing to be bound by theory, the applicant believes that the phosphopeptides slow the rate of growth of the metal nanoparticles, either by adsorbing onto the growing surface of the nanoparticles or by reducing the quantity of metal compound available, resulting in smaller nanoparticles.

The metal compound is reduced by the reducing agent in the presence of the phosphopeptide to provide the metal nanoparticle-phosphopeptide complex.

In one embodiment, the metal nanoparticle is an iron nanoparticle. In one embodiment, the iron nanoparticle is an iron-iron oxide core-shell nanoparticle. In one embodiment, the size of the iron-iron oxide core-shell nanoparticle is less than about 50 nm. In another embodiment, the size of the iron-iron oxide core-shell nanoparticle is less than about 30 nm. In another embodiment, the size of the iron-iron oxide core-shell nanoparticle is about 20 nm. In one embodiment, the size of the iron-iron oxide core-shell nanoparticle is from about 10 nm to about 50 nm. In one embodiment, the size of the iron-iron oxide core-shell nanoparticle is from about 8 nm to about 50 nm. In another embodiment, the size of the iron-iron oxide core-shell nanoparticle is from about 15 nm to about 25 nm. In one embodiment, the size of the iron-iron oxide core-shell nanoparticle is from about 8 nm to about 25 nm.

In one embodiment, the shell of the iron-iron oxide core-shell nanoparticle is about 5 nm.

In another embodiment, the iron nanoparticle is an iron oxide nanoparticle. In one embodiment, the size of the iron oxide nanoparticle is less than about 10 nm. In another embodiment, the iron oxide nanoparticle is about 8 nm.

In one embodiment, the metal nanoparticle is a cobalt nanoparticle.

In one embodiment, the metal nanoparticle is a nickel nanoparticle.

In one embodiment, the metal nanoparticle is a copper nanoparticle.

In one embodiment, the metal nanoparticle is a ruthenium nanoparticle. In one embodiment, the size of the ruthenium nanoparticle is from about 20 nm to 100 nm.

In one embodiment, the metal nanoparticle is a rhodium nanoparticle.

In one embodiment, the metal nanoparticle is a palladium nanoparticle. In one embodiment, the size of the palladium nanoparticle is from about 3 nm to about 7 nm. In another embodiment, the size of the palladium nanoparticle is about 5 nm.

In one embodiment, the metal nanoparticle is a silver nanoparticle.

In one embodiment, the metal nanoparticle is an iridium nanoparticle.

In one embodiment, the metal nanoparticle is a platinum nanoparticle.

In one embodiment, the metal nanoparticle is a gold nanoparticle. In one embodiment, the size of the gold nanoparticle is from about 3 nm to about 5 nm. In another embodiment, the size of the gold nanoparticle is about 4 nm.

In one embodiment, the metal nanoparticles produced by the methods have a relatively narrow size distribution. In one embodiment, the standard deviation of the particle size is less than the mean particle size. In another embodiment, the metal nanoparticles are substantially monodisperse.

The reducing agent is selected having regard to the nature of the metal compound. Examples of suitable reducing agents include but are not limited to citrate, hydrazine, bitartrate, carbon monoxide, ascorbic acid, hydrogen, metal hydrides, and the like.

In another embodiment, the reducing agent is a metal hydride. Examples of metal hydrides include lithium aluminium hydride, sodium hydride, potassium hydride, diisobutylaluminium hydride, and sodium borohydride.

In one embodiment, the metal hydride is a metal borohydride. In one embodiment, the metal borohydride is selected from the group consisting of lithium borohydride, sodium borohydride, sodium cyanoborohydride, potassium borohydride, and lithium triethylborohydride. In one embodiment, the metal borohydride is sodium borohydride.

In one embodiment, the liquid reaction medium comprises a solvent. In one embodiment, the solvent is selected from the group consisting of aqueous solvents, organic solvents, and mixtures thereof. Organic solvents include, but are not limited to, dimethyl formamide; dimethylsulfoxide; alcohols, for example methanol, ethanol, iso-propanol, and tert-butanol; ethers, for example tetrahydrofuran and diethyl ether; acetonitrile; nitromethane; chlorinated solvents, for example dichloromethane, chloroform, and carbon tetrachloride; aromatic solvents, for example benzene; and esters, for example ethyl acetate.

Advantageously, the methods can be carried out using non-toxic, aqueous or water miscible solvent systems.

In one embodiment, the solvent is an aqueous solution. In one embodiment, the aqueous solution is water.

In one embodiment, the liquid reaction medium comprises 5, 10, 15, 20, 25, 30, 35, 40, 45, 50, 55, 60, 65, 70, 75, 80, 85, 90, or 95% v/v water or more. In one embodiment, the liquid reaction medium is water.

The metal compound, reducing agent, and phosphopeptide may be contacted at any suitable temperature. In one embodiment, the metal compound, reducing agent, and phosphopeptide are contacted at ambient temperature. In another embodiment, the metal compound, reducing agent, and phosphopeptide are contacted at elevated temperature. In one embodiment, the elevated temperature is less than 200° C.

In one embodiment, the contacting step is carried out under an atmosphere of inert gas. In one embodiment, the inert gas is nitrogen or argon. Carrying out the reaction under an atmosphere of inert gas prevents the metal nanoparticles from being oxidising while they are growing. The metal nanoparticles may be oxidised on exposure to air.

Relatively small nanoparticles may be completely oxidised on exposure to air, while relatively large nanoparticles may only be partially oxidised. Oxidation of relatively large metal nanoparticles may result in the formation of metal-metal oxide core-shell nanoparticles.

In one embodiment, the methods further comprise mixing the metal compound, phosphopeptide, and reducing agent in the liquid reaction medium. Mixing the metal compound, phosphopeptide, and reducing agent in the liquid reaction medium ensures that the reaction mixture is homogeneous. The reaction mixture may be mixed by any method known in the art.

In one embodiment, the methods further comprise recovering the product metal nanoparticles or metal nanoparticle-phosphopeptide complex. Suitable methods include, but are not limited to, filtration, centrifugation, decanting, and magnetic separation. In one embodiment, the metal nanoparticles or metal nanoparticle-phosphopeptide complex is recovered by magnetic separation.

The recovered metal nanoparticles or metal nanoparticle-phosphopeptide complex may be further purified by, for example, washing the nanoparticles or nanoparticle-phosphopeptide complex in a suitable solvent. Suitable solvents include, but are not limited, water, ethanol, dimethyl sulfoxide, and dimethyl formamide.

The product metal nanoparticles or metal nanoparticle-phosphopeptide complexes may be stored in the form of powder. Conveniently, the powder may readily disperse when treated with a solvent to provide a stable suspension of the metal nanoparticles or metal nanoparticle-phosphopeptide complex in solution. Alternatively, the product metal nanoparticles or metal nanoparticle-phosphopeptide complexes may be stored in the form a suspension in a suitable solvent. Suitable solvents include, but are not limited to, water and ethanol.

In one embodiment, the metal nanoparticle exhibits super-paramagnetic behaviour at room temperature. In another embodiment, the metal nanoparticle exhibits ferrimagnetic behaviour at room temperature. In another embodiment, the metal nanoparticle exhibits ferromagnetic behaviour at room temperature.

In a further aspect, the present invention provides metal nanoparticles prepared by a method of the present invention.

In a further aspect, the present invention provides metal nanoparticle-phosphopeptide complex prepared by a method of the present invention.

In one embodiment, the metal nanoparticle is an iron nanoparticle. In one embodiment, the iron nanoparticles exhibit ferromagnetic, ferromagnetic, or superparamagnetic behaviour at room temperature.

In a further aspect, the present invention provides a method for preparing a metal nanoparticle-phosphopeptide complex, the method comprising contacting
a metal compound; and
a phosphopeptide comprising two or more contiguous peptide motifs and two or more phosphorus-containing groups capable of interacting with the surface of the metal nanoparticle,
   wherein the amino acids at the equivalent position in each peptide motif have similar structural and/or electronic properties, and
   wherein each phosphorus-containing group is bound to an amino acid in the two or more contiguous peptide motifs; and
      in a liquid reaction medium under conditions that precipitate a metal nanoparticle-phosphopeptide complex.

Various methods are available for forming metal nanoparitcles in solution by precipitation. Examples include, but are not limited to, formation of insoluble hydroxides, oxides, or sulfides, precipitation by addition of solvents in which the metal compound is insoluble or only sparingly soluble, and irradiation. A person skilled in the art will be able to select appropriate conditions having regard to the nature of the metal compound.

In one embodiment, the metal is as defined in any of the preceding embodiments.

In one embodiment, the method comprises contacting two or more metal compounds. In one embodiment, at least two of the two or more metal compounds comprise different metals.

In one embodiment, the method comprises co-precipitating two or more metal compounds in the presence of the phosphopeptide to form the metal nanoparticle phosphopeptide complex.

In one embodiment, at least one of the metal compounds comprises iron. Examples of metals suitable for co-precipitation with iron include but are not limited to cobalt and nickel.

In one embodiment, the metal compound is a metal salt. In one embodiment, the metal salt is as defined in any of the preceding embodiments.

In one embodiment, the metal nanoparticle is a metal oxide, metal hydroxide, or metal chalcogenide nanoparticle.

In one embodiment, the method comprises contacting one or more metal compounds, the phosphopeptide, and hydroxide or chalcogen ions. In one embodiment, the chalcogen ions are anions. In one embodiment, the chalcogen is sulfur. In one embodiment, sulfur anions are provided in the form of hydrogen sulfide.

In one embodiment, the conditions comprise a base.

In one embodiment, the metal nanoparticles are iron nanoparticles.

In a further aspect, the present invention provides a method for preparing iron nanoparticles, the method comprising contacting
iron (II);
iron (III);
a phosphopeptide comprising two or more contiguous peptide motifs and two or more phosphorus-containing groups capable of interacting with the surface of the iron nanoparticle,
   wherein the amino acids at the equivalent position in each peptide motif have similar structural and/or electronic properties, and
   wherein each phosphorus-containing group is bound to an amino acid in the two or more contiguous peptide motifs; and
a base
   in a liquid reaction medium to form iron nanoparticles.

In a further aspect, the present invention provides a method for preparing an iron nanoparticle-phosphopeptide complex, the method comprising contacting
iron (II);
iron (III);
a phosphopeptide comprising two or more contiguous peptide motifs and two or more phosphorus-containing groups capable of interacting with the surface of the iron nanoparticle,
   wherein the amino acids at the equivalent position in each peptide motif have similar structural and/or electronic properties, and
   wherein each phosphorus-containing group is bound to an amino acid in the two or more contiguous peptide motifs; and
a base
   in a liquid reaction medium to provide an iron nanoparticle-phosphopeptide complex.

The methods advantageously provide a one-pot route for producing metal nanoparticle-phosphopeptide complexes.

In one embodiment, the metal is as defined in any of the preceding embodiments.

In one embodiment, the metal is iron.

In one embodiment, iron (II) is formed in situ by the reduction of an iron (III) compound with a reducing agent, for example hydrogen or sodium borohydride. In another embodiment, iron (III) is formed in situ by the oxidation of an iron (II) compound with an oxidising agent, for example nitrate or oxygen.

In one embodiment, iron (II) is provided to the liquid reaction mixture in the form of an iron (II) compound and the iron (III) is provided to the liquid reaction mixture in the form of an iron (III) compound.

In one embodiment, the iron (II) compound is an iron (II) salt. In another embodiment, the iron (III) compound is an iron (III) salt. In one embodiment, the iron salts are selected from the group consisting of iron sulfates, iron acetoacetonates, iron oxalates, iron citrates, iron ammonium sulfates, iron sulfates, iron chlorides, and iron nitrates.

One embodiment utilises iron (II) sulfate and iron (III) chloride.

In one embodiment, the ratio of iron (II) to iron (III) is about 1:2.

The phosphopeptide affects the nucleation and growth of the metal nanoparticles in the liquid reaction medium. The phosphopeptide is as defined in any of the embodiments described herein.

Without wishing to be bound by theory, the applicant believes that the phosphopeptides slow the rate of growth of the metal nanoparticles, either by adsorbing onto the growing surface of the nanoparticles or by reducing the quantity of metal compound available, resulting in smaller nanoparticles.

In one embodiment, the method is for preparing iron nanoparticles or an iron nanoparticle-phosphopeptide complex. In one embodiment, the molar ratio of phosphorus-containing groups to iron is less than 1:1. In another embodiment, the molar ratio of phosphorus-containing groups to iron is from about 0.05:1 to about 0.95:1. In another embodiment, the molar ratio of phosphorus-containing groups to iron is from about 0.05:1 to about 0.75:1. In another embodiment, the molar ratio of phosphorus-containing groups to iron is from about 0.05:1 to about 0.5:1. In another embodiment, the molar ratio of phosphorus-containing groups to iron is from about 0.1:1 to about 0.4:1.

In one embodiment, the molar ratio of phosphorus-containing groups to iron (III) is less than 1:1. In another embodiment, the molar ratio of phosphorus-containing groups to iron (III) is from about 0.05:1 to about 0.95:1. In another embodiment, the molar ratio of phosphorus-containing groups to iron (III) is from about 0.05:1 to about 0.75:1. In another embodiment, the molar ratio of phosphorus-containing groups to iron (III) is from about 0.05:1 to about 0.5:1. In another embodiment, the molar ratio of phosphorus-containing groups to iron (III) is from about 0.1:1 to about 0.4:1.

The iron (II) and iron (III) coprecipitate in the presence of the base and the phosphopeptide to provide the iron nanoparticles or iron nanoparticle-phosphopeptide complex.

In one embodiment, the iron nanoparticle is an iron oxide nanoparticle. In one embodiment, the size of the iron oxide nanoparticle is less than about 10 nm. In another embodiment, the iron oxide nanoparticle is less than about 8 nm. In another embodiment, the iron oxide nanoparticle is about 5 nm.

In one embodiment, the metal nanoparticles produced by the methods have a relatively narrow size distribution. In one embodiment, the standard deviation of the particle size is less than the mean particle size. In another embodiment, the metal nanoparticles are substantially monodisperse.

Any suitable base may be used in the methods. In one embodiment, the base is ammonia. In another embodiment, the base is sodium hydroxide. In another embodiment, the base is an organic base. In one embodiment, the organic base is an organic amine. Examples of suitable organic amines include, but are not limited to, triethylamine, diisopropylethylamine

A person skilled in the art will appreciate that the coprecipitation reaction may be sensitive to pH and will be able to select appropriate bases for particular metal compounds.

In one embodiment, the liquid reaction medium comprises water. In one embodiment, the liquid reaction medium comprises 5, 10, 15, 20, 25, 30, 35, 40, 45, 50, 55, 60, 65, 70, 75, 80, 85, 90, or 95% v/v water or more. In one embodiment, the liquid reaction medium is water.

In one embodiment, the liquid reaction medium comprises a solvent. In one embodiment, the solvent is selected from the group consisting of aqueous solvents, organic solvents, and mixtures thereof. Organic solvents include, but are not limited to, dimethyl formamide; dimethylsulfoxide; alcohols, for example methanol, ethanol, iso-propanol, and tert-butanol; ethers, for example tetrahydrofuran and diethyl ether; acetonitrile; nitromethane; chlorinated solvents, for example dichloromethane, chloroform, and carbon tetrachloride; aromatic solvents, for example benzene; and esters, for example ethyl acetate.

Advantageously, the methods can be carried out using non-toxic, aqueous or water miscible solvent systems.

In one embodiment, the solvent is an aqueous solution. In one embodiment, the aqueous solution is water.

In one embodiment, the liquid reaction medium is water.

In one embodiment, the liquid reaction medium further comprises a buffer. A person skilled in the art will be able select an appropriate buffer considering the nature of components present in the liquid reaction medium and the desired pH, without undue experimentation.

In one embodiment, the contacting step is carried out at ambient temperature. In one embodiment, the contacting step is carried out under an atmosphere of inert gas. In one embodiment, the inert gas is nitrogen or argon. Carrying out the reaction under an atmosphere of inert gas prevents the metal nanoparticles from being oxidising while they are growing. The metal nanoparticles may be oxidised upon exposure to air.

In one embodiment, the methods further comprise mixing the metal compound, phosphopeptide, and base in the liquid reaction medium. Mixing the metal compound, phosphopeptide, and base in the liquid reaction medium ensures that the reaction mixture is homogeneous. The reaction mixture may be mixed by any method known in the art.

In one embodiment, the methods further comprise recovering the product metal nanoparticles or metal nanoparticle-phosphopeptide complex. Suitable methods include, but are not limited to, filtration, centrifugation, and decanting. In one embodiment, the metal nanoparticles or metal nanoparticle-phosphopeptide complex is recovered by magnetic separation.

The recovered metal nanoparticles or metal nanoparticle-phosphopeptide complex may be further purified by, for example, washing the nanoparticles or nanoparticle-phosphopeptide complex in a suitable solvent. Suitable solvents include, but are not limited, water, ethanol, and dimethyl sulfoxide, dimethyl formamide.

The product metal nanoparticles or metal nanoparticle-phosphopeptide complexes may be stored in the form of powder. Conveniently, the powder may readily disperse when treated with a solvent to provide a stable suspension of the metal nanoparticles or metal nanoparticle-phosphopeptide complex in solution. Suitable solvents include, but are not limited to, water, ethanol, dimethyl sulfoxide, and dimethyl formamide.

In one embodiment, the metal nanoparticle exhibits super-paramagnetic behaviour at room temperature. In another embodiment, the metal nanoparticle exhibits ferrimagnetic behaviour at room temperature. In another embodiment, the metal nanoparticle exhibits ferromagnetic behaviour at room temperature.

In one embodiment, the metal nanoparticles are iron nanoparticles. In one embodiment, the nanoparticles exhibit ferromagnetic, ferrimagnetic, or superparamagnetic behaviour at room temperature.

In a further aspect the present invention provides an metal nanoparticles prepared by a method of the present invention.

In a further aspect the present invention provides an metal nanoparticle-phosphopeptide complex prepared by a method of the present invention.

Advantageously, the metal nanoparticles in the metal nanoparticle-phosphopeptide complex prepared according to the methods of the present invention may exhibit superparamagnetic, ferromagnetic, and/or ferrimagnetic properties.

The metal nanoparticles and metal nanoparticle-phosphopeptide complexes of the present invention may be useful in medical applications, for example, the treatment of cancer by hyperthermia and as agents for contrast enhancement in medical imaging.

A person skilled in the art will be able to determine suitable doses of the metal nanoparticles or metal nanoparticle-phosphopeptide complex without undue experimentation.

The metal nanoparticles or metal nanoparticle-phosphopeptide complex may be formulated for administration by any method known in the art. Advantageously, the metal nanoparticles are and metal nanoparticle-phosphopeptide complex of the present invention may be capable of forming stable suspension in aqueous solutions.

Formulation of the metal nanoparticles or metal nanoparticle-phosphopeptide complex for use in medical applications, for example as a drug or contrast agent, can include binding an antibody to the nanoparticles. The presence of the phosphopeptides on the surface of the metal nanoparticles in the metal nanoparticle-phosphopeptide complex is a particular advantage for such procedures since the phosphopeptides can offer a range of chemical functionality that can be used for such binding. Methods for binding antibodies are well known in the art. EDC-NHS coupling of amino groups to carboxylic acid groups is one such method.

Formulation of the metal nanoparticles or metal nanoparticle-phosphopeptide complex for use in medical applications, for example as a drug or contrast agent, can also include binding compounds designed to minimise non-specific interactions of the particle with the surfaces of cells of the body, or to minimise inflammatory reactions. Such compounds are well-known in the art and include materials such as poly(ethyleneoxide), otherwise known as PEG. For example, amino-terminated PEG can be bound to chemical functionalities in the phosphopeptides of the nanoparticle-phosphopeptide complex using EDC-NHS coupling.

The present invention also provides various kits for preparing agents for use in treating cancer and in medical imaging as defined above.

The metal compound, phosphopeptide, and metal nanoparticle-phosphopeptide complex are as defined in any of the preceding embodiments.

Compounds for minimising non-specific interactions or inflammatory reactions will be apparent to the skilled worker. The specific compound used may depend on the intended application. Suitable compounds include, for example, PEG molecules.

Examples of targeting groups include antibodies, antibody fragments, singe chain antibodies, peptides, nucleic acids, carbohydrates, lipids, lectins, drugs, and any other compounds that bind to specifically targets in vivo. Other targeting groups will be apparent to those skilled in the art.

The coupling reagent, if necessary, depends on the nature of the compound and/or targeting group to be coupled and the specific reaction involved. For peptide couplings, numerous activating agents are commercially available.

The methods of the present invention for preparing metal nanoparticle-phosphopeptide complexes may conveniently provide metal nanoparticle-phosphopeptide complexes in a form suitable for administration without purification. For example, the preparation of iron nanoparticle-phosphopeptide complexes by reduction of iron (II) sulphate with sodium borohydride in water in the presence of a phosphopeptide rapidly provides an aqueous suspension of iron nanoparticle-phosphopeptide complexes and non-toxic by products (sodium borate). The iron nanoparticle-phosphopeptide complex can readily be coupled to, for example, various antibodies using standard techniques.

In a further aspect, the present invention provides a metal nanoparticle-phosphopeptide complex of the present invention for use as a catalyst.

In a further aspect, the present invention provides a catalyst comprising a metal nanoparticle-phosphopeptide complex of the present invention.

Advantageously, the metal nanoparticles of the present invention have significantly greater surface area than, for example, bulk metal. The increased surface area may provide enhanced activity in the catalysis of various desirable chemical reactions. The metal nanoparticles used depend on the reaction to be catalysed. Examples of possible catalyst applications include but are not limited to air cathodes in fuel cells, oxidation catalysts (e.g. for the conversion of CO to CO₂ in vehicle exhausts), and partial oxidation catalysts in various industrial reactions (e.g. for creating syngas).

The catalyst metal nanoparticles may be coated onto a support for use. Examples of suitable supports include but are not limited to ceramics and carbon (including both monolithic and particulate forms thereof).

The metal nanoparticle-phosphopeptide complexes have numerous other applications, as would be appreciated by a person skilled in the art. For example, silver metal nanoparticles may be useful as antimicrobial agents and metal chalcogenide nanoparticles may be useful as quantum dots (semiconducting nanoparticles with band gaps that are particle size and shape dependent and therefore tunable).

A person skilled in the art will appreciate that the optimum size of the metal nanoparticles in the metal nanoparticle in the present invention may vary depending on the intended application.

The following non-limiting examples are provided to illustrate the present invention and in no way limit the scope thereof.

### EXAMPLES

### General Information

All reagents were purchased as reagent grade and used without further purification. Solvents were used as supplied or dried according to standard protocols (Perrin, D. D. et al., Purification of Laboratory Chemicals, Pergamon Press Ltd., Oxford, 2^{nd} Ed., 1980). The progress of reactions was monitored by analytical thin layer chromatography (TLC) using 0.2 mm thick pre-coated silica gel plates (Merck Kieselgel 60 F₂₅₄ or Riedel-de Haen Kieselgel S F₂₅₄). Compounds were visualized by ultra-violet fluorescence or by staining with potassium permanganate solution, followed by heating the plate, as appropriate. Separation of mixtures was performed by flash chromatography using Merck Kieselgel 60 (230-400 mesh) with the indicated solvents Infrared spectra were obtained on an FTIR spectrometer as neat samples and absorption maxima are expressed in wavenumbers (cm⁻¹). ¹H NMR spectra were recorded on a Bruker AC 300 (300 MHz) spectrometer at ambient temperature. Chemical shifts are expressed in parts per million downfield from tetramethylsilane as an internal standard, and are reported as chemical shift (δ in ppm), relative integral, multiplicity, coupling constant (J in Hz) and assignment. ¹³C NMR spectra were recorded on a Bruker AC 300 (75 MHz) spectrometer at ambient temperature with complete proton decoupling. Electrospray ionization (ESI) mass spectra were recorded using a Thermo Finnigan Surveyor MSQPlus spectrometer, a Bruker micrOTOF-Q II spectrometer, or a hp Series 1100 MSD spectrometer. Transmission electron microscopy (TEM) images, electron diffraction patterns and energy dispersive spectroscopy (EDS) data were acquired digitally with a JEOL 2010 operated at an accelerating voltage of 200 KeV and equipped with an Oxford Inca EDS detector. The samples for TEM studies were prepared by resuspending the dry particles in ethanol using sonication, depositing a few drops of ethanol suspension on a copper or carbon-coated copper TEM grid, and allowing the ethanol to evaporate under ambient conditions. Magnetisation measurements were carried out on a superconducting quantum interference device (SQUID) magnetometer or a Quantum Design physical property measurement system (PPMS) using the Model P525 vibrating sample magnetometer (VSM) measurement system at 0 and 300 K. For the SQUID device, dry particles were weighted into a gelatin capsule which was then sealed and inserted in the SQUID sample holder for measurement. Dynamic light scattering measurements were carried out using a Malvern Zetasizer Nano ZS.

### Reagents

O-(Benzotriazol-1-yl)-N,N,N′,N′-tetramethyluronium hexafluorophosphate (HBTU) was purchased from Advanced ChemTech. N,N-Dimethylformamide (DMF) (synthesis grade), di-sodium hydrogen phosphate and acetonitrile (HPLC grade) were purchased from Scharlau. Piperidine, guanidine hydrochloride, 3,6-dioxa-1,8-octanedithiol, triisopropylsilane (TIS), 4-(dimethylamino)pyridine (DMAP), tris(2-carboxyethyl)phosphine hydrochloride (TCEP) and 4-methylmorpholine (NMM), stearic acid, lauric acid, Triton™ X-100 reduced and sodium borohydride (2M solution in triethylene glycol dimethyl ether) were purchased from Aldrich. N,N′-diisopropylcarbodiimide (DIC) was purchased from GL Biochem. Trifluoroacetic acid (TFA) was purchased from Halocarbon, CuSO₄.5H₂O from Ajax Finechem, copoly(styrene-1%-divinylbenzene) resin (Bio beads S-X1) 200-400 mesh from Bio-Rad, and TentaGel HL-NH₂ resin from Peptides International. All of the amino acids used were L-amino acids. Fmoc-Ala-OH, Fmoc-Asp(OtBu)-OH, Fmoc-Lys(Boc)-OH, Fmoc-Ser(OtBu)-OH, Fmoc-Glu(OtBu)-OH, and Fmoc-Arg(Pbf)-OH were purchased from either CEM corp. or GL Biochem. Fmoc-L-Ala-OCH₂PhOCH₂CH₂CO₂H and Fmoc-L-Lys(Boc)-OCH₂PhOCH₂CH₂CO₂H were purchased from PolyPeptide Group. Fmoc-L-propargylglycine (Pra) was synthesised according to published procedures (Lee, D. J. et al., Org. Lett., 2009, 11, 5270-5273; Hung, K.-y. et al., J. Org. Chem., 2010, 75, 8728-8731; Jensen, K. Jet al., J. Chem. Soc. Perkin Trans. 1, 1993, 2119-2129). Fmoc-Ala-Wang-Polystyrene resin was obtained from Advanced ChemTech. Fmoc-Ser(HPO₃Bn)-OH, Fmoc-Thr(HPO₃Bn)-OH and Fmoc-Tyr(HPO₃Bn)-OH were obtained from LC Sciences.

### Phosphopeptide 105

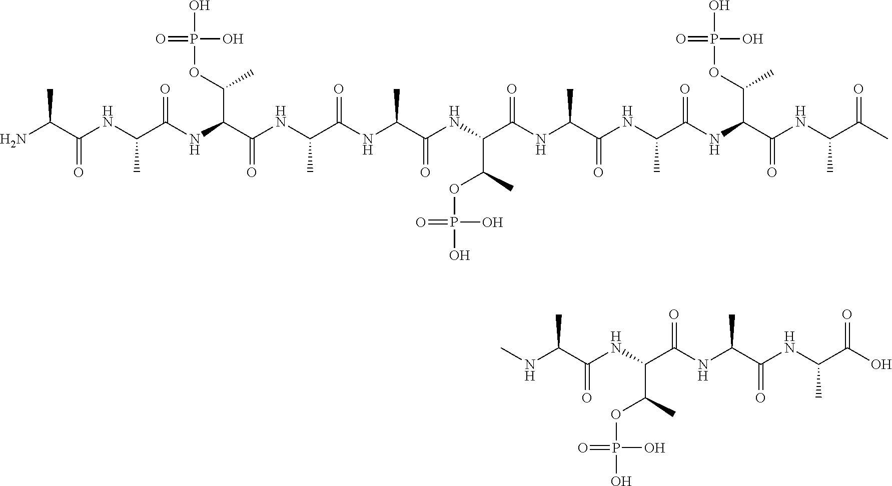

Solid phase peptide synthesis was performed using a Liberty Microwave Peptide Synthesizer (CEM Corporation, Mathews, N.C.), using the Fmoc/tBu strategy. Fmoc-Ala-Wang-Polystyrene resin was used as the starting material. Peptide synthesis was carried out at 0.1 mmol scale. Fmoc-Thr(HPO₃Bn)-OH was used for the coupling of phosphorylated threonine.

The Fmoc group was deprotected with 20% v/v piperidine in DMF for 30 seconds followed by a second deprotection for three minutes using a microwave power of 60 W for both deprotections. The maximum temperature for both deprotections was set at 75° C. Once a phosphorylated threonine was introduced in the peptide chain, the Fmoc deprotection was performed in the absence of microwave irradiation for 5 min and repeated for 15 min without microwave. The coupling steps were performed with 5 equivalents of Fmoc protected amino acid in DMF (0.2 M), 4.5 equivalents of HBTU in DMF (0.45 M) and 10 equivalents of NMM in DMF (2 M). Standard amino acid couplings were performed for five minutes at 25 W at a maximum temperature of 75° C. Fmoc-Thr(HPO₃Bn)-OH couplings were performed for 15 min at 25 W with a maximum temperature of 72° C. The amino acid immediately following the phosphorylated residue was also coupled for 15 min at 25 W with a maximum temperature of 72° C. Boc-Ala-OH was used as the last residue and coupled for five minutes at 25 W at a maximum temperature of 75° C.

Following completion of the sequence, the peptide was released from the resin with concomitant removal of protecting groups by treatment with TFA/TIPS/H₂O (95/2.5/2.5, v/v/v) at room temperature for three to five hours as required. The crude peptide was precipitated with cold diethyl ether, isolated by centrifugation, washed with cold diethyl ether, dissolved in 1:1 (v/v) acetonitrile:water containing 0.1% TFA and lyophilized.

The crude peptide product was analyzed for purity by analytical RP-HPLC (Dionex P680) at 210 and 254 nm using a Gemini C18 (4.60×250 mm, 110A, 50 column (Phenomenex) at 1 mL/min. The solvent system used was A (0.1% TFA in H₂O) and B (0.1% TFA in MeCN). Final purification was performed using Water 600 RP-HPLC using a Gemini C18 (10.00×250 mm, 110A, 50 column (Phenomenex). The solvent system used was A (0.1% TFA in H₂O) and B (0.1% TFA in MeCN). Final purity was determined by analytical RP-HPLC (Dionex P680) using the same conditions as for the crude product. Peptide mass was confirmed by LC-MS (Dionex Ultimate 3000 equipped with a Thermo Finnigan Surveyor MSQPlus spectrometer) using ESI in the positive mode: AATpAATpAATpAATpAA, wherein Tp=phosphorylated threonine, (2.9 mg, 2.0%): C₄₂H₇₆N₁₄O₃₁P₄; MW=1453.20 g.mol⁻¹; m/z (ESI) 1453.4 [M+H]⁺; 727.2 [M+2H]²⁺.

### Phosphopeptide 106

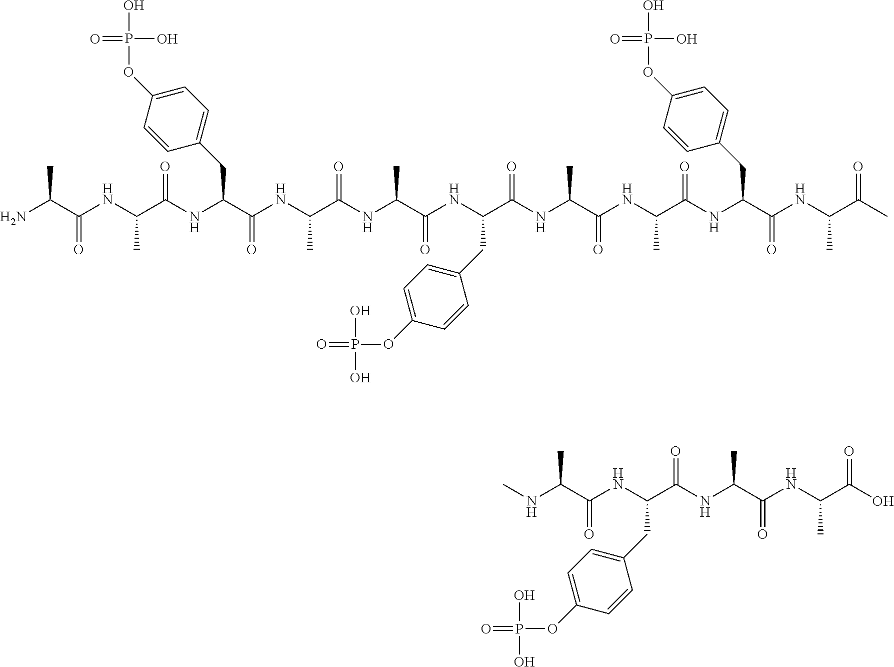 [SMILES: CC(=O)[C@H](C)NC(=O)[C@H](Cc1ccc(OP(=O)(O)O)cc1)NC(=O)[C@H](C)NC(=O)[C@H](C)NC(=O)[C@H](Cc1ccc(OP(=O)(O)O)cc1)NC(=O)[C@H](C)NC(=O)[C@H](C)NC(=O)[C@H](Cc1ccc(OP(=O)(O)O)cc1)NC(=O)[C@H](C)NC(=O)[C@H](C)N ; CN[C@@H](C)C(=O)N[C@@H](Cc1ccc(OP(=O)(O)O)cc1)C(=O)N[C@@H](C)C(=O)N[C@@H](C)C(=O)O]

Solid phase peptide synthesis was performed using a Tribute Peptide Synthesizer (Protein Technologies, Inc), using an Fmoc/tBu strategy. Fmoc-Ala-Wang-Polystyrene resin was used as the starting material. Peptide synthesis was carried out at 0.1 mmol scale. Fmoc-Tyr(HPO₃Bn)-OH was used for the coupling of phosphorylated tyrosine.

The Fmoc group was deprotected with 20% v/v piperidine in DMF for 5 min followed by a second deprotection for 15 min at room temperature. The coupling steps were performed with 5 equivalents of Fmoc protected amino acid in DMF (0.25 M), 4.5 equivalents of HBTU in DMF (0.24 M) and 10 equivalents of NMM in DMF (2 M). Standard amino acid couplings were performed for 40 min at room temperature. Fmoc-Tyr(HPO₃Bn)-OH couplings were performed for 1 h at room temperature. The amino acid immediately following each phosphorylated amino acid was also coupled for 1 h at room temperature. Fmoc-Ala-OH was used as the last residue and coupled for 40 min at room temperature.

The peptide was deprotected and released from the resin, and the crude peptide isolated, analyzed, and purified as described above for the synthesis of phosphopeptide 105: AAYpAAYpAAYpAAYpAA, wherein Yp=phosphorylated tyrosine, (2.7 mg, 1.6%): C₄₂H₇₆N₁₄O₃₁P₄; MW=1701.48 g.mol⁻¹; m/z (ESI) 1702.3 [M+H]⁺; 851.2 [M+2H]²⁺.

### Phosphopeptide 107

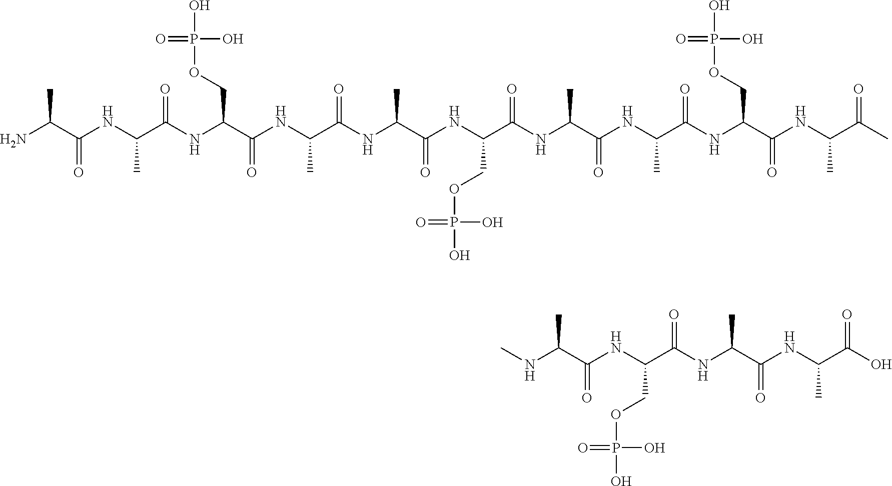

Phosphopeptide 107 was prepared using a procedure analogous to that described above for the preparation of phosphopeptide 106, using Fmoc-Ser(HPO₃Bn)-OH instead of Fmoc-Tyr(HPO₃Bn)-OH: AASpAASpAASpAASpAA, wherein Sp=phosphorylated serine, (3.8 mg, 2.7%): C₄₂H₇₆N₁₄O₃₁P₄; MW=1397.2 g.mol⁻¹; m/z (ESI) 1397.36 [M+H]⁺; 699.19 [M+2H]²⁺.

Phosphopeptide 107 was also prepared using a procedure analogous to that described above for the preparation of phosphopeptide 106, using Fmoc-Ser(HPO₃Bn)-OH instead of Fmoc-Tyr(HPO₃Bn)-OH and using Boc-Ala-OH as the last amino acid instead of Fmoc-Ala-OH: (6.0 mg, 4.3%): C₄₂H₇₆N₁₄O₃₁P₄; MW=1397.2 g.mol⁻¹; m/z (ESI) 1397.34 [M+H]⁺; 699.20 [M+2H]²⁺.

Phosphopeptide 107 was also prepared using Fmoc-Ala-Wang-Tentagel resin as the starting material instead of Fmoc-Ala-Wang-Polystyrene resin. Fmoc-Ala-Wang-Tentagel resin was prepared by adding Fmoc-Ala-Wang-linker-OH (Polypeptide Group) (0.2 mM) in DCM (3 mL) and DIC (42 mL) to 0.1 mM of TentaGel S NH₂ resin (RAPP Polymere) previously swollen in DCM. The suspension was shaken gently for 1 h, the resin washed with DCM and dried under nitrogen.

Peptide synthesis was carried out at 0.1 mmol scale. Fmoc-Ser(HPO₃Bn)-OH was used for the coupling of phosphorylated serine.

The Fmoc group was deprotected with 20% v/v piperidine in DMF for 5 min followed by a second deprotection for 15 min at room temperature. The coupling steps were performed with 5 equivalents of Fmoc protected amino acid in DMF (0.25 M), 4.5 equivalents of HBTU in DMF (0.24 M) and 10 equivalents of NMM in DMF (2 M). Standard amino acid couplings were performed for 40 min at room temperature. Fmoc-Ser(HPO₃Bn)-OH couplings were performed for 1 h at room temperature. The amino acid immediately following each phosphorylated amino acid was also coupled for 1 h at room temperature. Boc-Ala-OH was used as the last residue and coupled for 40 min at room temperature.

The peptide was deprotected and released from the resin, and the crude peptide isolated, analyzed, and purified as described above for the synthesis of phosphopeptide 105: (9.9 mg, 7.1%): C₄₂H₇₆N₁₄O₃₁P₄; MW=1397.2 g.mol⁻¹; m/z (ESI) 1397.3 [M+H]⁺; 699.2 [M+2H]²⁺.

### Phosphopeptide 108

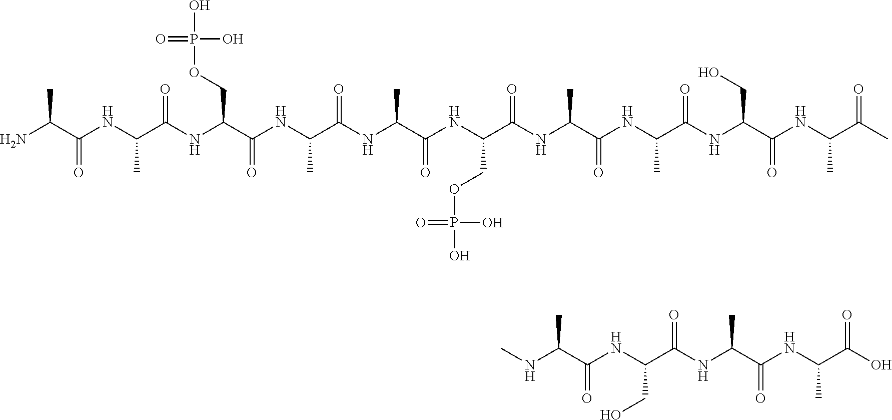

Solid phase peptide synthesis was performed using a Tribute Peptide Synthesizer (Protein Technologies, Inc), using an Fmoc/tBu strategy. Fmoc-Ala-Wang-Tentagel resin was used as the starting material. Peptide synthesis was carried out at 0.1 mmol scale. Fmoc-Ser(HPO₃Bn)-OH was used for the coupling of phosphorylated serine.

The Fmoc group was deprotected with 20% v/v piperidine in DMF for 5 min followed by a second deprotection for 15 min at room temperature. The coupling steps were performed with 5 equivalents of Fmoc protected amino acid in DMF (0.25 M), 4.5 equivalents of HBTU in DMF (0.24 M) and 10 equivalents of NMM in DMF (2 M). Standard amino acid couplings were performed for 40 min at room temperature. Fmoc-Ser(HPO₃Bn)-OH couplings were performed for 1 h at room temperature. The amino acid immediately following each phosphorylated amino acid was also coupled for 1 h at room temperature. Boc-Ala-OH was used as the last residue and coupled for 40 min at room temperature.

The peptide was deprotected and released from the resin, and the crude peptide isolated, analyzed, and purified as described above for the synthesis of phosphopeptide 105: AASpAASpAASAASAA, wherein Sp=phosphorylated serine, (12.0 mg, 9.7%): C₄₂H₇₄N₁₄O₂₅P₂; MW=1237.06 g.mol⁻¹; m/z (ESI) 1237.44 [M+H]⁺; 619.22 [M+2H]²⁺.

### Phosphopeptide 109

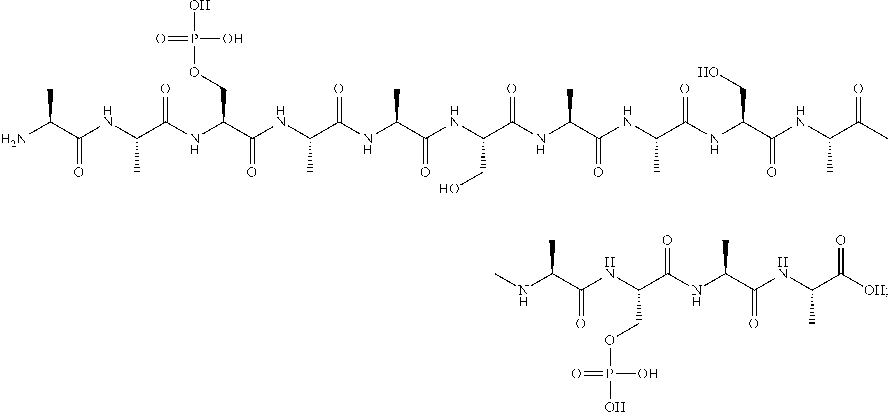

Phosphopeptide 109 was prepared using a procedure analogous to that described above for the preparation of phosphopeptide 108: AASpAASAASAASpAA, wherein Sp=phosphorylated serine, (3.0 mg, 2.4%): C₄₂H₇₄N₁₄O₂₅P₂; MW=1237.06 g.mol⁻¹; m/z (ESI) 1237.41 [M+H]⁺; 619.22 [M+2H]²⁺.

### Phosphopeptide 110

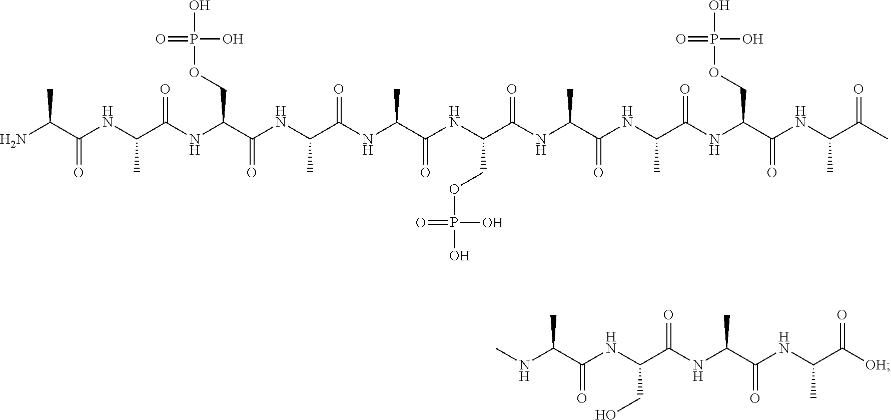

Phosphopeptide 110 was prepared using a procedure analogous to that described above for the preparation of phosphopeptide 108: AASpAASpAASpAASAA, wherein Sp=phosphorylated serine, (13.6 mg, 10.3%): C₄₂H₇₅N₁₄O₂₈P₃; MW=1317.04 g.mol⁻¹; m/z (ESI) 1317.41 [M+H]⁺; 659.21 [M+2H]²⁺.

### 3-O-(Phospho)-L-serine 112

Commercially available Fmoc-Ser(HPO₃Bn)-OH (200 mg, 0.4 mmol) was dissolved in a mixture of 20% diethylamine in DMF (2 mL) and stirred for two hours at room temperature. The solvent was then removed under reduced pressure. The mixture was then dissolved in methanol (2 mL), 10% Pd/C (10 mg) was added and the reaction was left to stir overnight with H₂ gas bubbling into the mixture. The suspension was then filtered through on Celite® and the solvent evaporated under reduced pressure. After suspension in water (3 mL), the suspension was washed with diethyl ether (2×3 mL) and ethyl acetate (2×3 mL). The aqueous phase was collected and the water evaporated under reduced pressure. The resulting residue was recrystallised from a mixture of water, cold diethyl ether and methanol to afford 112 (44 mg, 60%) as a white powder. IR vmax (neat) 3002 (br), 1629, 1516, 1088, 987, 760; ¹11 NMR (300 MHz, D₂O) δ 4.18-4.06 (2H, m, H(3), 3.94-3.91 (1H, m, Hβ); m/z (ESI) 184.00 [M−H]⁻, 369.01 [M₂−H]⁻. The ¹H NMR, IR and MS data obtained were in agreement with that reported in the literature (Arnold, L. D. et al. J. Am. Chem. Soc. 1988, 110, 2237-2241).

### Peptide 113

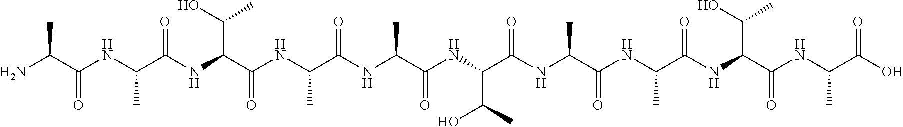 [SMILES: C[C@H](N)C(=O)N[C@@H](C)C(=O)N[C@H](C(=O)N[C@@H](C)C(=O)N[C@@H](C)C(=O)N[C@H](C(=O)N[C@@H](C)C(=O)N[C@@H](C)C(=O)N[C@H](C(=O)N[C@@H](C)C(=O)O)[C@@H](C)O)[C@@H](C)O)[C@@H](C)O]

Solid phase peptide synthesis was performed using a Liberty Microwave Peptide Synthesizer (CEM Corporation, Mathews, N.C.), using the Fmoc/tBu strategy. Fmoc-Ala-Wang-Polystyrene resin was used as the starting material. Peptide synthesis was carried out at 0.1 mmol scale.

The Fmoc group was deprotected with 20% v/v piperidine in DMF for 30 seconds followed by a second deprotection for three minutes using a microwave power of 60 W for both deprotections. The maximum temperature for both deprotections was set at 75° C. The coupling steps were performed with 5 equivalents of Fmoc protected amino acid in DMF (0.2 M), 4.5 equivalents of HBTU in DMF (0.45 M) and 10 equivalents of DIPEA in N-methylpyrrolidone (2 M) Amino acid couplings were performed for 5 min at 25 W at a maximum temperature of 75° C.

The peptide was deprotected and released from the resin, and the crude peptide isolated.

Final purification was performed using RP-HPLC (Water 600LCD) using a Jupiter C18 (10.00×250 mm, 300 Å, 5μ) column (Phenomenex). The solvent system used was A (0.1% TFA in H₂O) and B (0.1% TFA in MeCN). Final purity was determined by analytical RP-HPLC (Dionex P680) at 210 and 254 nm using an Aqua C18 (4.60×250 mm, 125 Å, 5μ) column (Phenomenex) at 1 mL/min using a linear gradient. The solvent system used was A (0.1% TFA in H₂O) and B (0.1% TFA in MeCN). Peptide mass was confirmed by LC-MS (Dionex Ultimate 3000 equipped with a Thermo Finnigan Surveyor MSQPlus spectrometer) using ESI in the positive mode: AATAATAATA, (21.0 mg, 26%); C₃₃H₅₈N₁₀O₁₄; MW=818.9 g.mol⁻¹; m/z (ESI) 819.4 [M+H]⁺; 1638.3 [M+2H]²⁺.

### Peptide 77

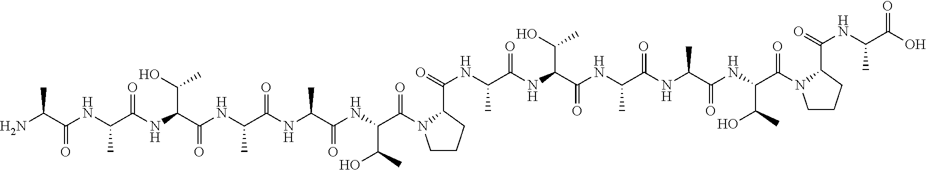 [SMILES: C[C@H](N)C(=O)N[C@@H](C)C(=O)N[C@H](C(=O)N[C@@H](C)C(=O)N[C@@H](C)C(=O)N[C@H](C(=O)N1CCC[C@H]1C(=O)N[C@@H](C)C(=O)N[C@H](C(=O)N[C@@H](C)C(=O)N[C@@H](C)C(=O)N[C@H](C(=O)N1CCC[C@H]1C(=O)N[C@@H](C)C(=O)O)[C@@H](C)O)[C@@H](C)O)[C@@H](C)O)[C@@H](C)O]

Peptide 77 was prepared using a procedure analogous to that described above for the preparation of peptide 113.

Following completion of the sequence the peptide was released from the resin with concomitant removal of protecting groups by treatment with TFA/TIPS/H₂O (95/2.5/2.5, v/v/v) either at room temperature for two to three hours as required or under microwave irradiation for 18 min at 10 W with a maximum temperature of 35° C. The crude peptide was precipitated with cold diethyl ether, isolated by centrifugation, washed with cold diethyl ether, dissolved in 1:1 (v/v) acetonitrile:water containing 0.1% TFA and lyophilised.

The crude peptide was analysed for purity by analytical RP-HPLC (Dionex P680) at 210 and 254 nm using an Aqua C18 (4.60×250 mm, 125 Å, 5μ) column (Phenomenex) at 1 mL/min using a linear gradient of 1% to 95% B over 30 min. The solvent system used was A (0.1% TFA in H₂O) and B (0.1% TFA in MeCN). Final purification was performed using RP-HPLC (either Water 600LCD or Gilson 281) using a Jupiter C18 (10.00×250 mm, 300 Å, 50 column (Phenomenex) at 5 ml/min using a linear gradient. The solvent system used was A (0.1% TFA in H₂O) and B (0.1% TFA in MeCN). Final purity was determined by analytical RP-HPLC (Dionex P680) at 210 and 254 nm using an Aqua C18 (4.60×250 mm, 125 Å, 50 column (Phenomenex) at 1 mL/min using a linear gradient. Again, the solvent system used was A (0.1% TFA in H₂O) and B (0.1% TFA in MeCN). Peptide mass was confirmed by LC-MS (Dionex Ultimate 3000 equipped with a Thermo Finnigan Surveyor MSQPlus spectrometer) using ESI in the positive mode: AATAATPATAATPA, (37 mg, 31%):

C₅₀H₈₄N₁₄O₁₉; MW=1185.3 g.mol⁻¹; m/z (ESI) 593.4 [M+2H]²⁺.

### Synthesis of 2-azidoethylphosphonic acid 116

### (i) Diethyl 2-azidoethylphosphonate 115

Diethyl 2-bromoethylphosphonate (500 mg, 2 mmol) was dissolved in DCM (10 mL) with tetrabutylammonium bisulfate (2.04 g, 6 mmol). A solution of sodium azide (1.30 g, 20 mmol) in distilled water was added (10 mL) and the resulting mixture was stirred vigorously in a sealed flask. The reaction progress was monitored by TLC. After 6 days, the product was extracted with diethyl ether (2×10 mL), dried over Na₂SO₄, filtered, and the solvent evaporated. The crude residue was purified via flash chromatography (1:1→3:1 EtOAc-hexanes) to afford the title compound (370 mg, 89%) as a white solid. R_{f}=0.225 (2:1 EtOAc-hexanes); IR vmax (neat) 2099, 1240, 1019, 957; ¹11 NMR (400 MHz, CDCl₃) δ 1.34 (6H, t, J=6.9 Hz, CH₃), 2.07 (2H, dt, J=18.6 Hz, J=7.8 Hz, CH₂), 3.55 (2H, dt, J=12.6 Hz, J=7.5 Hz, CH₂), 4.13 (4H, m, CH₂); ¹³C NMR (400 MHz, D₂O) δ 16.1, 24.72, 26.58, 45.13, 61.60; m/z (ESI) 208.08 [M+H]⁺, 230.07 [M+Na]⁺. The ¹H and ¹³C NMR and MS data obtained were in agreement with that reported in the literature (Brunet, E. et al. Tetrahedron Lett. 2009, 50, 5361-5363).

### (ii) 2-Azidoethylphosphonic acid 116

Diethyl 2-azidoethylphosphonate (370 mg, 1.8 mmol) was dissolved in DMF (2.5 mL) and the solution was cooled to −5° C. Trimethylbromosilane (12.5 mL, 7.1 mmol) was then added and the solution left to stir overnight at room temperature. The solvent was evaporated and co-evaporated with dry toluene (3×5 mL). The residue was redissolved in water (10 mL), stirred overnight at room temperature then concentrated and concentrated with water (3×10 mL). The residue was then dissolved in water and freeze-dried to afford the title compound (200 mg, 74%) as an oil. No further purification was carried out and the crude product was used directly for the click reaction onto the peptide. IR vmax (neat) 2778 (br), 2098, 1115, 927; ¹H NMR (400 MHz, D₂O) δ 1.85 (2H, dt, J=18.1 Hz, J=7.1 Hz, CH₂), 3.30 (2H, dt, J=15.7 Hz, J=7.1 Hz, CH₂); m/z (ESI) 150.14 [M−H]⁻, 301.29 [M₂−H]⁻. The ¹H NMR and MS data obtained were in agreement with that reported in the literature (Alexandrova, L. A. et al. Nucleic Acids Res. 1998, 26, 778-786).

### Synthesis of Phosphopeptide 111

### (i) Peptide 117

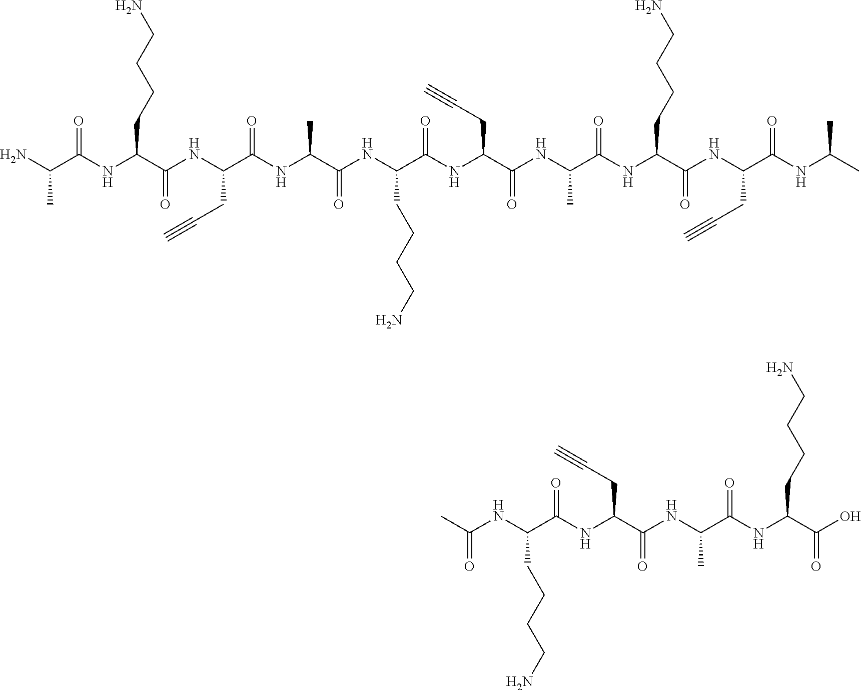

Peptide 117 was prepared using a procedure analogous to that described above for the preparation of phosphopeptide 105, using Fmoc-Lys-Wang-Polystyrene resin as the starting material instead of Fmoc-Ala-Wang-Polystyrene resin and using Fmoc-Gly(Propagyl)-OH instead of Fmoc-Thr (HPO₃Bn)-OH: AKPraAKPraAKPraAKPraAK, wherein Pra=propargyl glycine.

Peptide 117 was also prepared by the following method.

Solid phase peptide synthesis based on Fmoc protection strategy was performed on a 0.1 mmol scale using aminomethylated polystyrene resin (Mitchell, A. R. et al., J. Org. Chem., 1978, 43(14), 2845-2852) (loading 1.0 mmol/g). The aminomethylated resin was swollen in DCM (5 mL) for 15 min and then the solvent was drained. Fmoc-L-Lys(Boc)-OCH₂PhOCH₂CH₂CO₂H (2 eq) was dissolved in 1 mL of DCM, DIC (2 eq) was added and the reaction mixture was added to resin followed by agitating for 1 h. The mixture was drained and the resin was washed with DMF (3×) and DCM (3×).

The peptide chain was assembled by manual SPPS.

N^{α}-Protected amino acids Fmoc-Ala-OH and Fmoc-Lys(Boc)-OH (5 eq) were dissolved in 2 mL of 0.23 M HBTU/DMF (4.6 eq), 0.5 ml of 2M NMM/DMF (10 eq) were added and the mixture was transferred to the reaction vessel. The mixture was shaken for 45 min, filtered and washed with DMF (3×) and DCM (3×). The N^{α}-protecting group was removed by 20% piperidine solution in DMF (3 mL, 2×5 min), filtered and washed with DMF (3×) and DCM (3×). The N^{α}-protecting group was removed by 20% piperidine solution in DMF (3 mL, 2×5 min), filtered and washed with DMF (3×) and DCM (3×).

N^{α}-Protected Fmoc-L-propargylglycine (50 mg, 0.15 mmol, 1.5 eq), HATU (55 mg, 0.145 mmol, 1.45 eq) and HOAt (20 mg, 0.145 mmol, 1.45 eq) were dissolved in 2 mL of DMF, 2,4,6-collidine (80 μL, 0.6 mmol, 6 eq) and DMAP (cat., 10 μL of a stock solution of 1.22 mg DMAP in 122 μL DMF) were added and the mixture was transferred to the reaction vessel. The mixture was shaken for 1 h, filtered and washed with DMF (3×) and DCM (3×). The N^{α}-protecting group was removed by 20% piperidine solution in DMF (3 mL, 2×5 min), filtered and washed with DMF (3×) and DCM (3×).

Following completion of the sequence, the peptide was cleaved from the resin with concomitant removal of the protecting groups by treating the resin with 100 μL TIPS, 250 μL H₂O, 250 μL 3,6-dioxa-1,8-octanedithiol and 9.4 mL TFA and agitating the mixture for 2 h at room temperature. The TFA solution was filtered and the peptide was precipitated by addition of hexane/diethyl ether (1:1). After centrifugation and washing with hexane/diethyl ether (1:1) the peptide was lyophilised from 0.1% trifluoroacetic acid-water to yield 167.5 mg of crude AKPraAKPraAKPraAKPraAK, wherein Pra=propargylglycine. m/z (ESI-MS): [M+2H⁺] calculated mass=698.2, observed mass=698.0; [M+3H⁺] calculated mass=465.8, observed mass=465.8; [M+4H⁺] calculated mass=349.6, observed mass=349.8; [M+5H⁺] calculated mass=279.9, observed mass=280.0.

### (ii) Phosphopeptide 111

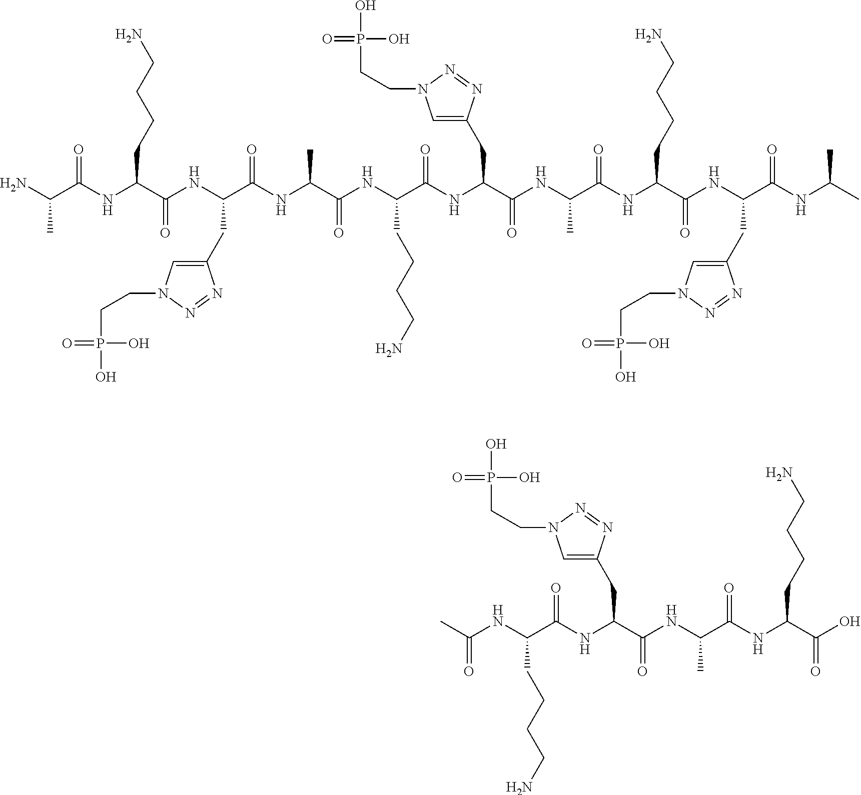

Crude propargylated peptide 117 (23.2 mg, 0.0166 mmol, 3 eq) was dissolved in a degassed aqueous solution of 6 M GnHCl/0.2 M Na₂HPO₄ (5.533 mL) containing TCEP hydrochloride (885.6 μL of 0.5 M aqueous solution, pH=7, 80 eq) and CuSO₄.5H₂O (885.6 μL of 0.5 M aqueous solution, 80 eq). After 30 min of incubation at 55° C., 2-azidoethylphosphonic acid 116 (15.04 mg, 0.0996 mmol, 18 eq) was added (752 μL of a stock solution of 20 mg 2-azidoethylphosphonic acid 116 in 1 mL H₂O) and the reaction carried out under argon with microwave irradiation in a sealed glass reaction vessel on a CEM Discover 908010 microwave reactor with IR-monitored temperature control (20 W, 60° C., 1 h). The reaction mixture was acidified to pH=1 with conc. HCl, purified by solid phase extraction using an Alltech C18-LP 900 mg bed cartridge and lyophilised.

The peptide was purified by semi-preparative RP-HPLC on a Dionex Ultimate 3000 system using a Phenomenex Gemini C₁₈, 5 μm, 10 mm×250 mm column and a linear gradient of 0.1% trifluoroacetic acid-water (A) and 0.1% trifluoroacetic acid-acetonitrile (B) at a flow rate of 5 mL/min, 0% to 51% B over 33 min, with detection at 210 nm. Lyophilisation yielded purified phosphopeptide 111 (9.44 mg, 29%) as a white solid in ca. 99% purity according to analytical HPLC on a Dionex P680 system using a Waters XTerra MS C₁₈, 5 μm, 4.6 mm×150 mm column. Rₜ 9.59 min (0-40% B over 16 min, 1 mL/min); m/z (ESI-MS): [M+2H⁺] calculated mass=1000.5, observed mass=1000.0; [M+3H⁺] calculated mass=667.3, observed mass=667.0; [M+4H⁺] calculated mass=500.7, observed mass=500.6; [M+5H⁺] calculated mass=400.8, observed mass=400.6.

### Synthesis of Phosphopeptide 201

### (i) Peptide 200

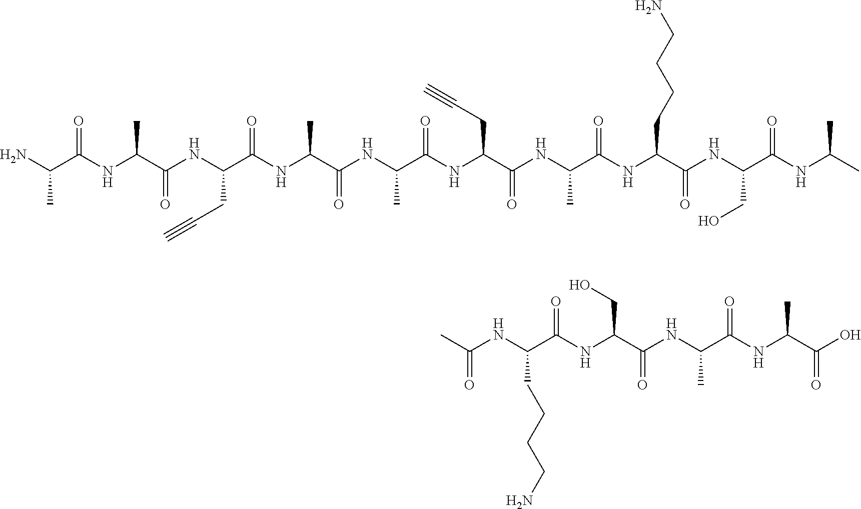

Solid phase peptide synthesis based on Fmoc protection strategy was performed on a 0.1 mmol scale using aminomethylated polystyrene resin (Mitchell, A. R. et al., J. Org. Chem., 1978, 43(14), 2845-2852) (loading 1.0 mmol/g). The aminomethylated resin was swollen in DCM (5 mL) for 15 min and then the solvent was drained. Fmoc-L-Ala-OCH₂PhOCH₂CH₂CO₂H (2 eq) was dissolved in 1 mL of DCM, DIC (2 eq) was added and the reaction mixture was added to resin followed by agitating for 1 h. The mixture was drained and the resin was washed with DMF (3×) and DCM (3×).

Residues AKSAKSA of the peptide chain were assembled by automated SPPS using a Tribute™ peptide synthesizer and the Fmoc/tBu strategy.

N^{α}-Protected amino acids Fmoc-Ala-OH and Fmoc-Lys(Boc)-OH (5 eq) were dissolved in 2 mL of 0.23 M HBTU/DMF (4.6 eq), 0.5 ml of 2M NMM/DMF (10 eq) were added and the mixture was transferred to the reaction vessel. The mixture was shaken for 45 min, filtered and washed with DMF (3×) and DCM (3×). The N^{α}-protecting group was removed by 20% piperidine solution in DMF (3 mL, 2×5 min), filtered and washed with DMF (3×) and DCM (3×).

N^{α}-Protected Fmoc-L-propargylglycine (50 mg, 0.15 mmol, 1.5 eq), HATU (55 mg, 0.145 mmol, 1.45 eq) and HOAt (20 mg, 0.145 mmol, 1.45 eq) were dissolved in 2 mL of DMF, 2,4,6-collidine (80 μL, 0.6 mmol, 6 eq) and DMAP (cat., 10 μL of a stock solution of 1.22 mg DMAP in 122 μL DMF) were added and the mixture was transferred to the reaction vessel. The mixture was shaken for 1 h, filtered and washed with DMF (3×) and DCM (3×). The N^{α}-protecting group was removed by 20% piperidine solution in DMF (3 mL, 2×5 min), filtered and washed with DMF (3×) and DCM (3×).

Residues AAPraAAPra of the peptide chain were assembled by manual SPPS using the Fmoc/tBu strategy.

Couplings of N^{α}-Fmoc-protected amino acids (5 eq) were carried out in 45 min at room temperature in the presence of HBTU (4.6 eq) and NMM (10 eq) in DMF. The N^{α}-protecting group was removed by 20% piperidine solution in DMF (3 mL, 2×5 min).

Following completion of the sequence, the peptide was cleaved from the resin with concomitant removal of the protecting groups by treating the resin with 100 μL TIPS, 250 μL H₂O, 250 μL 3,6-dioxa-1,8-octanedithiol and 9.4 mL TFA and agitating the mixture for 2 h at room temperature. The TFA solution was filtered and the peptide was precipitated by addition of hexane/diethyl ether (1:1). After centrifugation and washing with hexane/diethyl ether (1:1) the peptide was lyophilised from 0.1% trifluoroacetic acid-water to yield 122.0 mg of crude AAPraAAPraAKSAKSAA. m/z (ESI-MS): [M+2H⁺] calculated mass=604.6, observed mass=604.8; [M+3H⁺] calculated mass=403.4, observed mass=403.4.

### (ii) Phosphopeptide 201

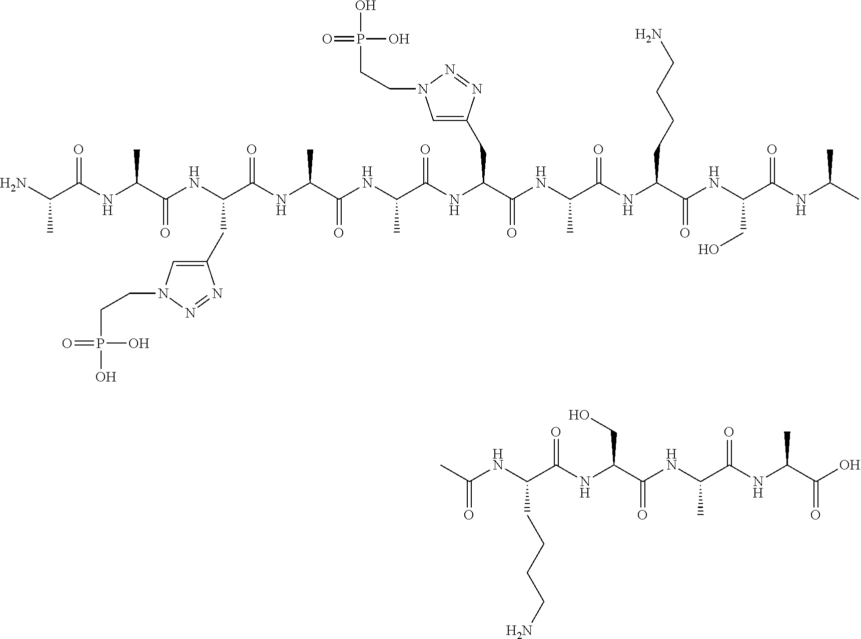

Crude propargylated peptide 200 (20.0 mg, 0.0166 mmol, 3 eq) was dissolved in a degassed aqueous solution of 6 M GnHCl/0.2 M Na₂HPO₄ (5.533 mL) containing TCEP hydrochloride (442.6 μL of 0.5 M aqueous solution, pH=7, 40 eq) and CuSO₄.5H₂O (442.6 μL of 0.5 M aqueous solution, 40 eq). After 30 min of incubation at 55° C., 2-azidoethylphosphonic acid (7.52 mg, 0.0498 mmol, 9 eq) was added (376 μL of a stock solution of 20 mg 2-azidoethylphosphonic acid in 1 mL H₂O) and the reaction carried out under argon with microwave irradiation in a sealed glass reaction vessel on a CEM Discover 908010 microwave reactor with IR-monitored temperature control (20 W, 60° C., 1 h). The reaction mixture was acidified to pH=1 with conc. HCl, purified by solid phase extraction and lyophilised.

The phosphopeptide was purified as described above for phosphopeptide 111. Lyophilisation yielded the purified phosphopeptide 201 (7.90 mg, 32%) as a white solid in ca. 95% purity according to analytical HPLC on a Dionex P680 system using a Waters XTerra MS C₁₈, 5 μm, 4.6 mm×150 mm column. Rₜ 10.50 min (0-40% B over 16 min, 1 mL/min); m/z (ESI-MS): [M+H⁺] calculated mass=1510.5, observed mass=1509.6; [M+2H⁺] calculated mass=755.7, observed mass=755.4; [M+3H⁺] calculated mass=504.2, observed mass=504.0.

### Synthesis of phosphopeptide 203

### (i) Peptide 202

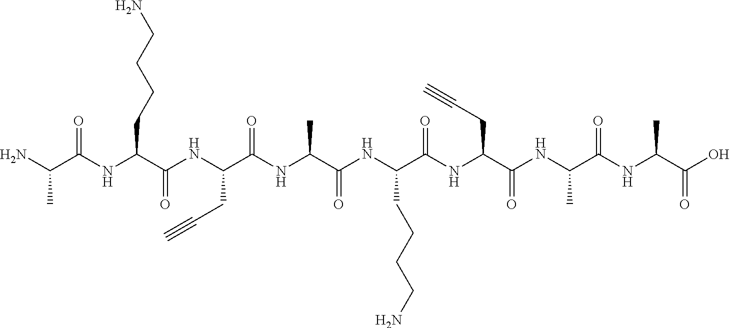 [SMILES: C#CC[C@H](NC(=O)[C@H](CCCCN)NC(=O)[C@H](C)N)C(=O)N[C@@H](C)C(=O)N[C@@H](CCCCN)C(=O)N[C@@H](CC#C)C(=O)N[C@@H](C)C(=O)N[C@@H](C)C(=O)O]

Peptide 202 was prepared using a procedure analogous to that described above for the preparation of peptide 200, using only manual SPPS to assemble the peptide chain. Lyophilisation yielded 87.0 mg of crude AKPraAKPraAA. m/z (ESI-MS): [M+H⁺] calculated mass=749.8, observed mass=749.4; [M+2H⁺] calculated mass=375.4, observed mass=375.2.

### (ii) Phosphopeptide 203

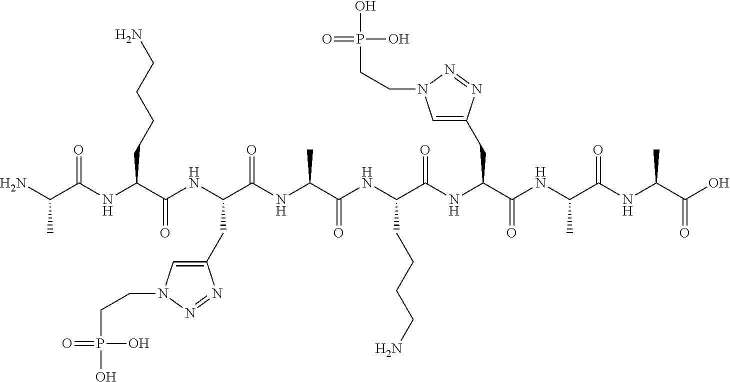 [SMILES: C[C@H](N)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](C)C(=O)O]

Crude propargylated peptide 202 (12.4 mg, 0.0166 mmol, 3 eq) was dissolved in a degassed aqueous solution of 6 M GnHCl/0.2 M Na₂HPO₄ (5.533 mL) containing TCEP hydrochloride (885.2 μL of 0.5 M aqueous solution, pH=7, 80 eq) and CuSO₄.5H₂O (885.2 μL of 0.5 M aqueous solution, 80 eq). After 30 min of incubation at 55° C., 2-azidoethylphosphonic acid (15.04 mg, 0.0996 mmol, 18 eq) was added (752 μL of a stock solution of 20 mg 2-azidoethylphosphonic acid in 1 mL H₂O) and the reaction carried out under argon with microwave irradiation in a sealed glass reaction vessel on a CEM Discover 908010 microwave reactor with IR-monitored temperature control (20 W, 60° C., 2 h). The reaction mixture was acidified to pH=1 with conc. HCl, purified by solid phase extraction and lyophilised.

The phosphopeptide was purified as described above for phosphopeptide 111, using a linear gradient of 0% to 50% B over 40 min instead of 0% to 51% B over 33 minutes. Lyophilisation yielded the purified phosphopeptide 203 (3.54 mg, 20%) as a white solid in ca. 94% purity according to analytical HPLC on a Dionex P680 system using a Waters XTerra MS C₁₈, 5 μm, 4.6 mm×150 mm column. Rₜ 10.38 min (0-40% B over 16 min, 1 mL/min); m/z (ESI-MS): [M+2H⁺] calculated mass=526.5, observed mass=526.6.

### Synthesis of phosphopeptide 205

### (i) Peptide 204

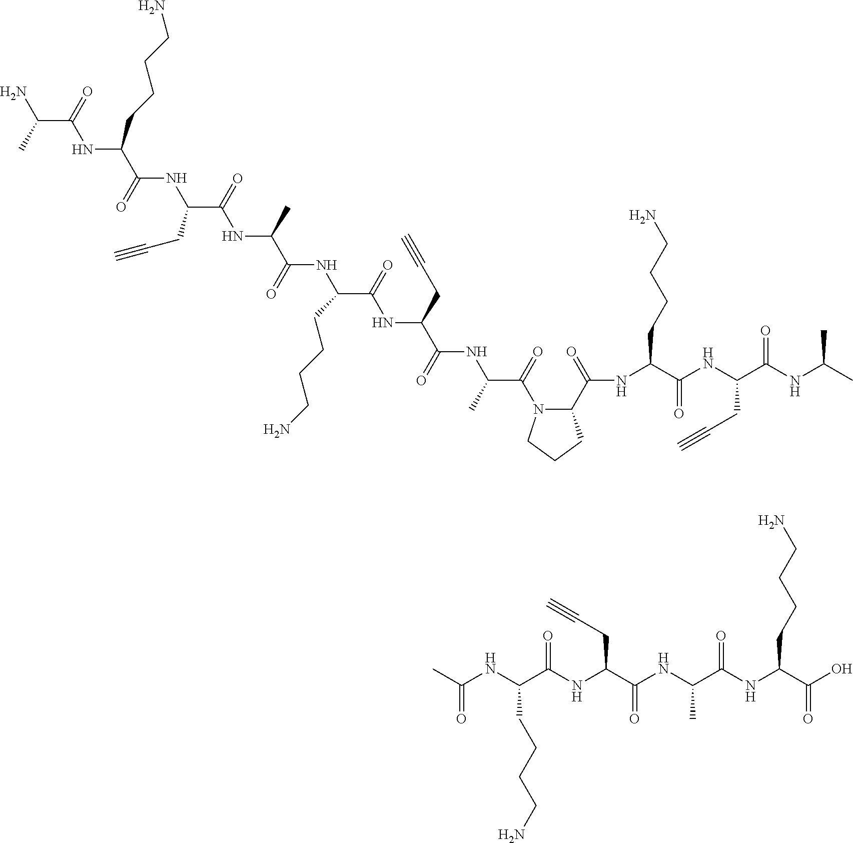

Peptide 204 was prepared using a procedure analogous to that described above for the preparation of peptide 200, using Fmoc-L-Lys(Boc)-OCH₂PhOCH₂CH₂CO₂H, instead of Fmoc-L-Ala-OCH₂PhOCH₂CH₂CO₂H, and using only manual SPPS to assemble the peptide chain. Lyophilisation yielded 187.2 mg of crude AKPraAKPraAPKPraAKPraAK. m/z (EST-MS): [M+H⁺] calculated mass=1492.6, observed mass=1491.9; [M+2H⁺] calculated mass=746.8, observed mass=746.4; [M+3H⁺] calculated mass=498.2, observed mass=498.0; [M+4H⁺] calculated mass=373.9, observed mass=373.7.

### (ii) Phosphopeptide 205

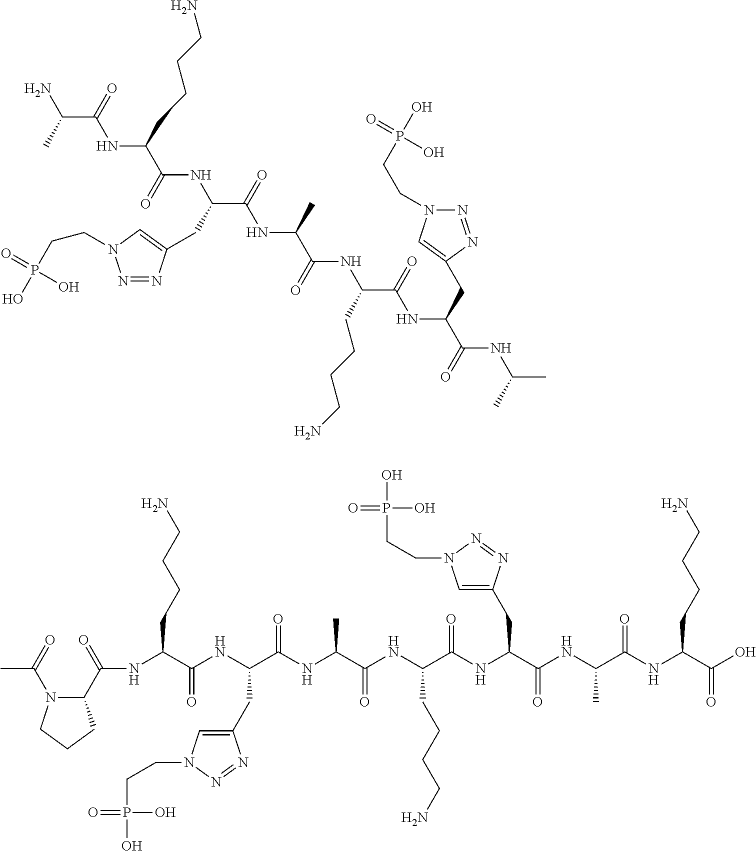

Crude propargylated peptide 204 (24.8 mg, 0.0166 mmol, 3 eq) was dissolved in a degassed aqueous solution of 6 M GnHCl/0.2 M Na₂HPO₄ (5.533 mL) containing TCEP hydrochloride (1327.8 μL of 0.5 M aqueous solution, pH=7, 120 eq) and CuSO₄.5H₂O (1327.8 μL of 0.5 M aqueous solution, 120 eq). After 30 min of incubation at 55° C., 2-azidoethylphosphonic acid (22.56 mg, 0.1494 mmol, 27 eq) was added (1.128 mL of a stock solution of 20 mg 2-azidoethylphosphonic acid in 1 mL H₂O) and the reaction carried out under argon with microwave irradiation in a sealed glass reaction vessel on a CEM Discover 908010 microwave reactor with IR-monitored temperature control (20 W, 60° C., 2 h). The reaction mixture was acidified to pH=1 with conc. HCl, purified by solid phase extraction and lyophilised.

The phosphopeptide was purified as described above for phosphopeptide 111, using a linear gradient of 0% to 50% B over 40 min instead of 0% to 51% B over 33 minutes. Lyophilisation yielded the purified phosphopeptide 205 (10.40 mg, 30%) as a white solid in ca. 98% purity according to analytical HPLC on a Dionex P680 system using a Waters XTerra MS C₁₈, 5 μM, 4.6 mm×150 mm column. Rₜ 11.065 min (0-40% B over 16 min, 1 mL/min); m/z (ESI-MS): [M+3H⁺] calculated mass=699.7, observed mass=699.8; [M+4H⁺] calculated mass=525.0, observed mass=525.1.

### Synthesis of phosphopeptide 207

### (i) Peptide 206

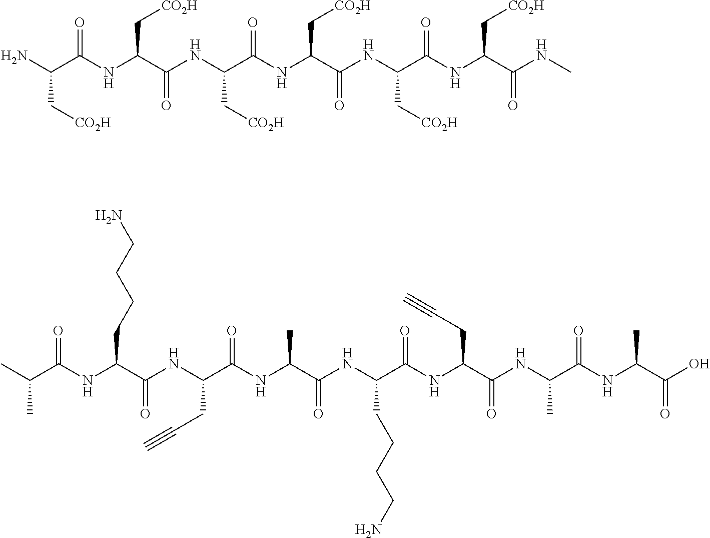

Peptide 206 was prepared using a procedure analogous to that described above for the preparation of peptide 200, using manual SPPS to assemble residues AKPraAKPraA of the peptide chain and automated SPPS to assemble residues DDDDDD. Lyophilisation yielded 152.6 mg of crude 206. m/z (ESI-MS): [M+H⁺] calculated mass=1440.3, observed mass=1440.3; [M+2H⁺] calculated mass=720.7, observed mass=720.6.

A portion of the crude peptide (30 mg) was purified as described above for phosphopeptide 111, using a linear gradient of 0% to 50% B over 20 min instead of 0% to 51% B over 33 minutes. Lyophilisation yielded the purified peptide DDDDDDAKPraAKPraAA (10.10 mg, 36%) as a white solid in ca. 99% purity according to analytical HPLC on a Dionex P680 system using a Waters XTerra MS C₁₈, 5 μm, 4.6 mm×150 mm column. Rₜ 11.65 min (0-40% B over 16 min, 1 mL/min)

### (ii) Phosphopeptide 207

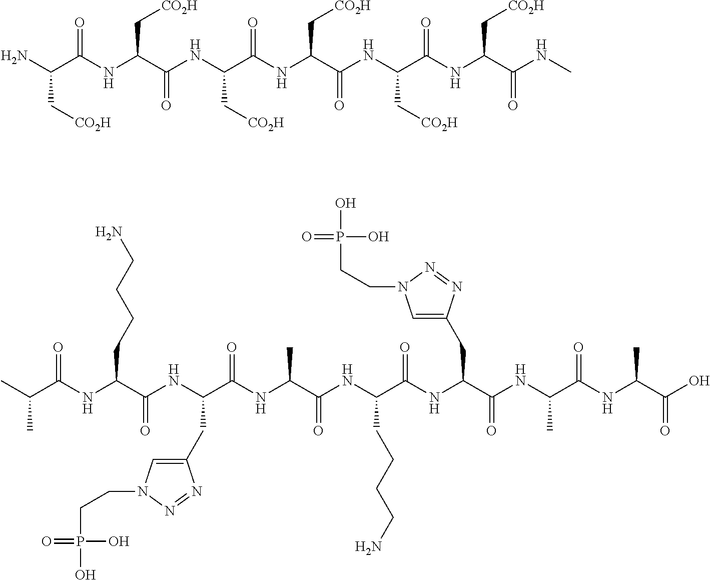

Crude propargylated peptide 206 (10.0 mg, 0.00695 mmol, 3 eq) was dissolved in a degassed aqueous solution of 6 M GnHCl/0.2 M Na₂HPO₄ (2.317 mL) containing TCEP hydrochloride (370 μL of 0.5 M aqueous solution, pH=7, 80 eq) and CuSO₄.5H₂O (370 μL of 0.5 M aqueous solution, 80 eq). After 30 min of incubation at 55° C., 2-azidoethylphosphonic acid (6.30 mg, 0.0417 mmol, 18 eq) was added (315 μL of a stock solution of 20 mg 2-azidoethylphosphonic acid in 1 mL H₂O) and the reaction carried out under argon with microwave irradiation in a sealed glass reaction vessel on a CEM Discover 908010 microwave reactor with IR-monitored temperature control (20 W, 60° C., 2 h). The reaction mixture was acidified to pH=1 with conc. HCl, purified by solid phase extraction and lyophilised.

The phosphopeptide was purified as described above for phosphopeptide 111, using a linear gradient of 0% to 50% B over 20 min instead of 0% to 51% B over 33 minutes. Lyophilisation yielded the purified phosphopeptide 207 (1.56 mg, 13%) as a white solid in ca. 95% purity according to analytical HPLC on a Dionex P680 system using a Waters XTerra MS C₁₈, 5 μm, 4.6 mm×150 mm column. Rₜ 10.79 min (0-40% B over 16 min, 1 mL/min); m/z (ESI-MS): [M+2H⁺] calculated mass=871.8, observed mass=872.0.

### Synthesis of phosphopeptide 209

### (i) Peptide 208

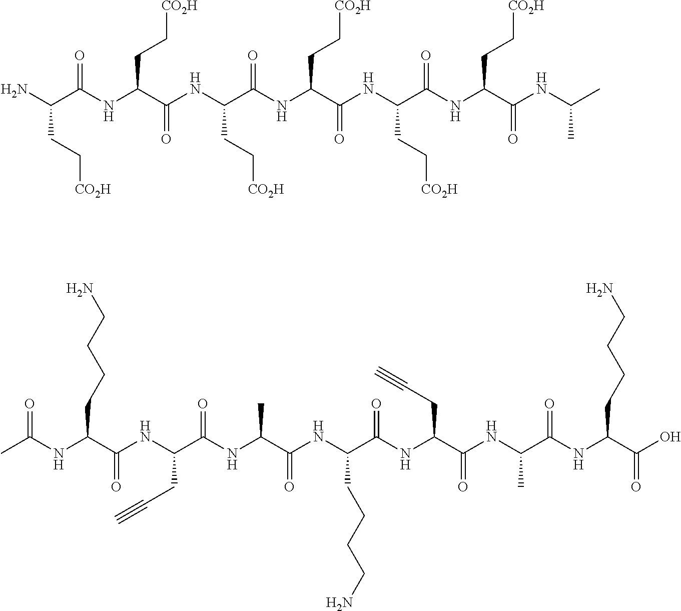

Peptide 208 was prepared using a procedure analogous to that described for peptide 200, using Fmoc-L-Lys(Boc)-OCH₂PhOCH₂CH₂CO₂H instead of Fmoc-L-Ala-OCH₂PhOCH₂CH₂CO₂H, a 0.05 mmol scale instead of 0.01 mmol, and using only manual SPPS to assemble the peptide chain. Lyophilisation yielded 80.9 mg of crude EEEEEEAKpraAKpraAK. m/z (ESI-MS): [M+H⁺] calculated mass=1581.6, observed mass=1581.7; [M+2H⁺] calculated mass=791.3, observed mass=790.9; [M+3H⁺] calculated mass=527.9, observed mass=527.6.

### (ii) Phosphopeptide 209

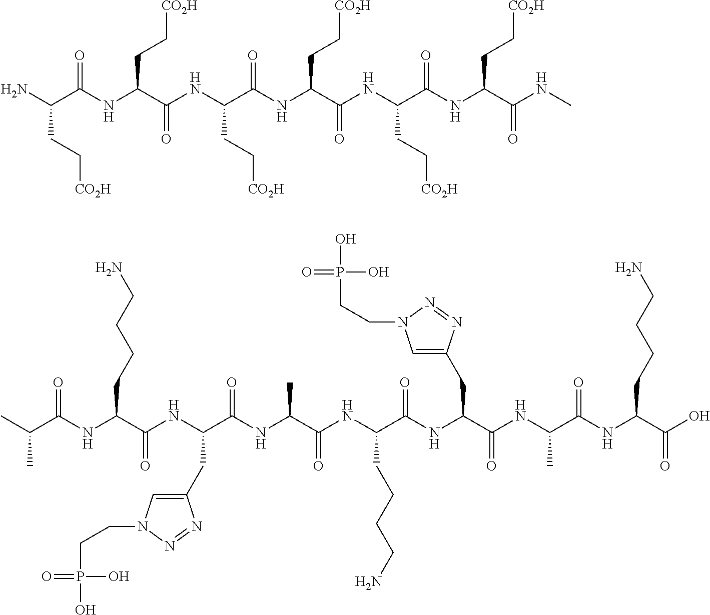

Crude propargylated peptide 208 (26.2 mg, 0.0166 mmol, 3 eq) was dissolved in a degassed aqueous solution of 6 M GnHCl/0.2 M Na₂HPO₄ (5.533 mL) containing TCEP hydrochloride (885.2 μL of 0.5 M aqueous solution, pH=7, 80 eq) and CuSO₄.5H₂O (885.2 μL of 0.5 M aqueous solution, 80 eq). After 30 min of incubation at 55° C., 2-azidoethylphosphonic acid (15.05 mg, 0.0996 mmol, 18 eq) was added (1.505 mL of a stock solution of 10 mg 2-azidoethylphosphonic acid in 1 mL H₂O) and the reaction carried out under argon with microwave irradiation (20 W, 60° C., 2 h). The reaction mixture was acidified to pH=1 with conc. HCl, purified by solid phase extraction and lyophilised.

The phosphopeptide was purified as described for phosphopeptide 111 using a linear gradient of 0% to 50% B over 20 min instead of 0% to 51% B over 33 minutes. Lyophilisation yielded the purified phosphopeptide 209 (7.50 mg, 24%) as a white solid in ca. 95% purity according to analytical HPLC on a Dionex P680 system using a Waters XTerra MS C₁₈, 5 μm, 4.6 mm×150 mm column. Rₜ 11.2 min (0-40% B over 16 min, 1 mL/min); m/z (ESI-MS): [M+2H⁺] calculated mass=942.4, observed mass=942.5; [M+3H⁺] calculated mass=628.6, observed mass=628.9; [M+4H⁺] calculated mass=471.7, observed mass=471.9.

### Synthesis of phosphopeptide 211

### (i) Peptide 210

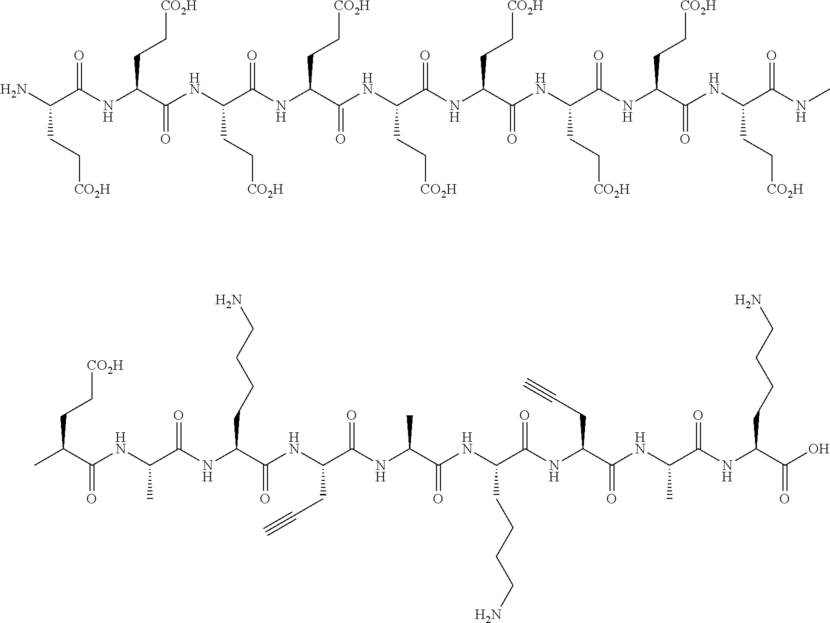

Peptide 210 was prepared using a procedure analogous to that described for peptide 208. Lyophilisation yielded 146.0 mg of crude EEEEEEEEEEAKpraAKpraAK. m/z (ESI-MS): [M+2H⁺] calculated mass=1049.5, observed mass=1049.5; [M+3H⁺] calculated mass=700.0, observed mass=700.0.

### (ii) Phosphopeptide 211

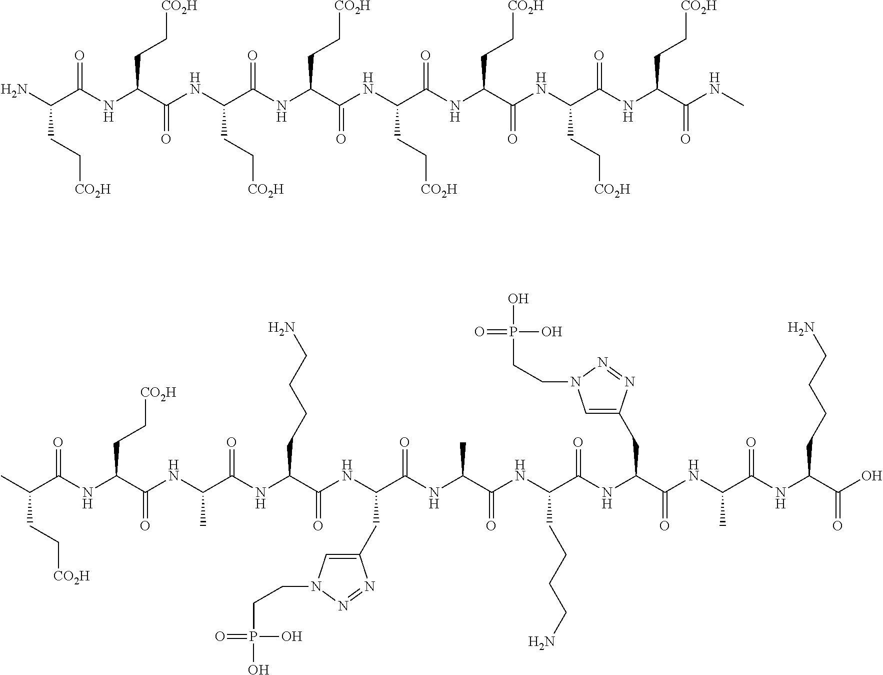

Crude propargylated peptide 210 (34.8 mg, 0.0166 mmol, 3 eq) was dissolved in a degassed aqueous solution of 6 M GnHCl/0.2 M Na₂HPO₄ (5.533 mL) containing TCEP hydrochloride (885.2 μL of 0.5 M aqueous solution, pH=7, 80 eq) and CuSO₄.5H₂O (885.2 μL of 0.5 M aqueous solution, 80 eq). After 30 min of incubation at 55° C., 2-azidoethylphosphonic acid (15.05 mg, 0.0996 mmol, 18 eq) was added (1.505 mL of a stock solution of 10 mg 2-azidoethylphosphonic acid in 1 mL H₂O) and the reaction carried out under argon with microwave irradiation (20 W, 60° C., 2 h). The reaction mixture was acidified to pH=1 with conc. HCl, purified by solid phase extraction and lyophilised.

The phosphopeptide was purified as described for phosphopeptide 111 by semi-preparative RP-HPLC using a linear gradient of 0% to 50% B over 20 min instead of 0% to 51% B over 33 minutes. Lyophilisation yielded the purified phosphopeptide (2.80 mg, 7%) as a white solid in ca. 88% purity according to analytical HPLC on a Dionex P680 system using a Waters XTerra MS C₁₈, 5 μm, 4.6 mm×150 mm column. Rₜ 16.6 min (0-40% B over 16 min, 1 mL/min); m/z (ESI-MS): [M+3H⁺] calculated mass=800.8, observed mass=801.0; [M+4H⁺] calculated mass=600.8, observed mass=601.2.

### Synthesis of phosphopetides 300-304

### (i) Peptides 300-302 and Phosphopeptide 303

Peptides 300-303 were prepared by the following general procedure.

Solid phase peptide synthesis based on Fmoc/tBu strategy was performed on a 0.1 mmol scale using aminomethylated polystyrene resin or TentaGel HL-NH₂ resin derivatised with Fmoc-L-Ala-OCH₂Ph-OCH₂CH₂CO₂H or Fmoc-L-Lys(Boc)-OCH₂PhOCH₂CH₂CO₂H.

The peptide chains were assembled using either manual Fmoc SPPS or a Tribute™ peptide synthesiser as described for peptide 200, standard amino acids or the building blocks Fmoc-L-propargylglycine and Fmoc-Ser(HPO₃Bn)-OH(Sₚ).

Peptides 300-301 were capped with lauric acid (300) and stearic acid (301) (0.5 mmol fatty acid, 0.46 mmol HATU, 0.46 HOAt, 1 mmol 2,4,6,-collidine) for 1 h. All peptides were cleaved from the resin with concomitant removal of the protecting groups as described for peptide 200 above.

Peptides 300-302 were carried through to the next step without further purification. Final purification and characterisation of peptide 303 was performed using RP-HPLC and LC-MS.

Semi-preparative RP-HPLC was performed as described for peptide 111. Analytical RP-HPLC was performed as described for peptide 111, using the Phenomenex Gemini C₁₈, 3 μm, 4.6 mm×150 mm column at a flow rate of 1 mL/min.

### Peptide 300

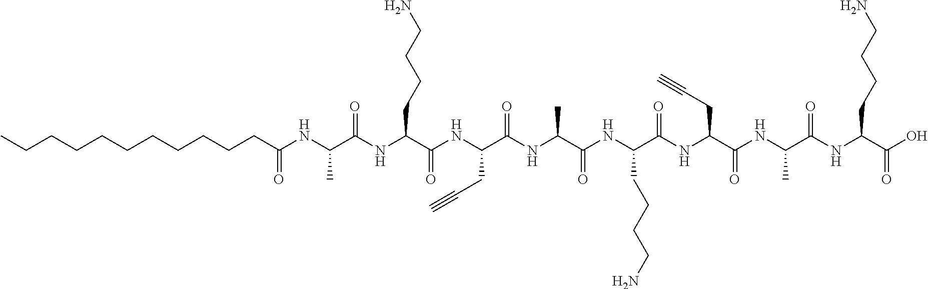 [SMILES: C#CC[C@H](NC(=O)[C@H](CCCCN)NC(=O)[C@H](C)NC(=O)[C@H](CC#C)NC(=O)[C@H](CCCCN)NC(=O)[C@H](C)NC(=O)CCCCCCCCCCC)C(=O)N[C@@H](C)C(=O)N[C@@H](CCCCN)C(=O)O]

Peptide 300 was prepared using a procedure analogous to that described for peptide 200, using Fmoc-L-Lys(Boc)-OCH₂PhOCH₂CH₂CO₂H instead of Fmoc-L-Ala-OCH₂PhOCH₂CH₂CO₂H, and using only manual SPPS to assemble the peptide chain. Laurie acid was attached as described above. Lyophilisation yielded 115.4 mg of crude lauric acid-AKpraAKpraAK. m/z (ESI-MS): [M+H⁺] calculated mass=990.2, observed mass=989.6; [M+2H⁺] calculated mass=495.6, observed mass=495.0; [M+3H⁺] calculated mass=330.7, observed mass=330.6.

### Peptide 301

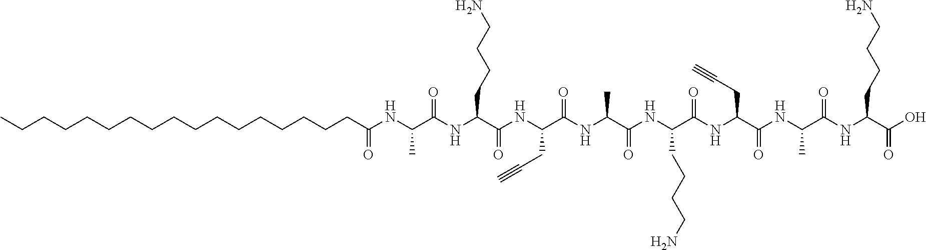 [SMILES: C#CC[C@H](NC(=O)[C@H](CCCCN)NC(=O)[C@H](C)NC(=O)[C@H](CC#C)NC(=O)[C@H](CCCCN)NC(=O)[C@H](C)NC(=O)CCCCCCCCCCCCCCCCC)C(=O)N[C@@H](C)C(=O)N[C@@H](CCCCN)C(=O)O]

Peptide 301 was prepared using a procedure analogous to that described for peptide 200, using Fmoc-L-Lys(Boc)-OCH₂PhOCH₂CH₂CO₂H instead of Fmoc-L-Ala-OCH₂PhOCH₂CH₂CO₂H, and using only manual SPPS to assemble the peptide chain. Stearic acid was attached as described above. Lyophilisation yielded 134.2 mg of crude stearic acid-AKpraAKpraAK. m/z (ESI-MS): [M+2H⁺] calculated mass=536.7, observed mass=537.4.

### Peptide 302

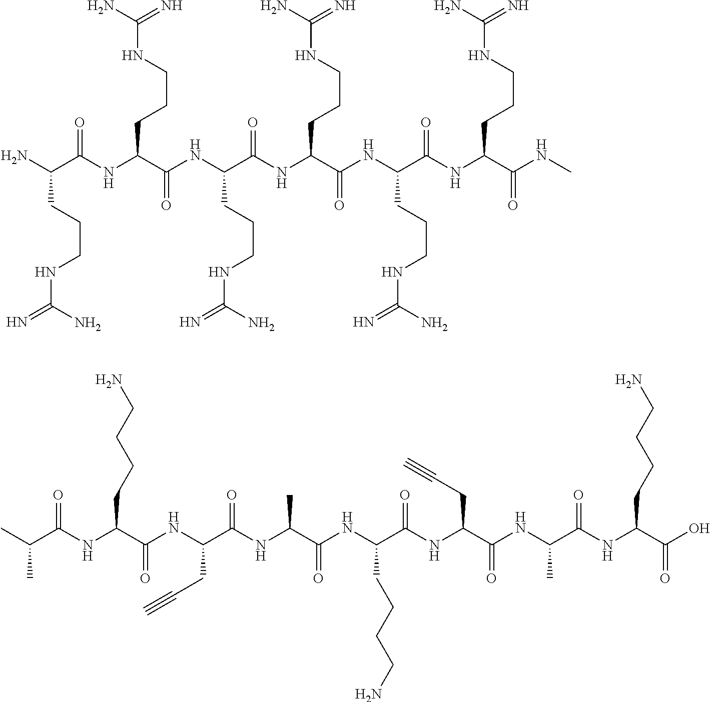

Peptide 302 was prepared using a procedure analogous to that described above for the preparation of peptide 200, using Fmoc-L-Lys(Boc)-OCH₂PhOCH₂CH₂CO₂H, instead of Fmoc-L-Ala-OCH₂PhOCH₂CH₂CO₂H and using manual SPPS to assemble residues AKPraAKPraA of the peptide chain and automated SPPS to assemble residues RRRRRR. Lyophilisation yielded 81.4 mg of crude RRRRRRAKpraAKpraAA. m/z (ESI-MS):

[M+3H⁺] calculated mass=582.0, observed mass=581.4; [M+4H⁺] calculated mass=436.8, observed mass=436.7.

### Phosphopeptide 303

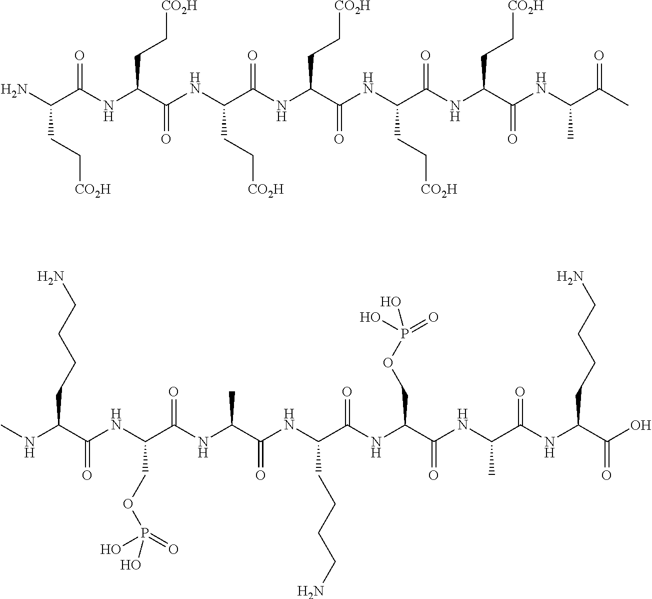

Peptide 303 was prepared using a procedure analogous to that described above for the preparation of peptide 200, using Fmoc-L-Lys(Boc)-OCH₂PhOCH₂CH₂CO₂H, instead of Fmoc-L-Ala-OCH₂PhOCH₂CH₂CO₂H, TentaGel HL-NH₂ resin instead of aminomethylated PS resin and using only automated SPPS (coupling time: 1 h) to assemble the peptide chain. Lyophilisation yielded 145.7 mg of crude EEEEEEAKSₚAKSₚAK.

A portion of the crude peptide (21.9 mg) was purified using a linear gradient of 0% to 40% B over 20 min instead of 0% to 51% B over 33 minutes. Lyophilisation yielded the purified peptide EEEEEEAKSₚAKSₚAK (3.7 mg, 14%) as a white solid in ca. 98% purity according to analytical HPLC. Rₜ 13.8 min (0-40% B over 15 min, 1 mL/min).

### (ii) Phosphopeptides 304-306

Peptides 304-306 were prepared from crude peptides 300-302, respectively, by the following general procedure.

Microwave-enhanced click reactions were performed in a sealed glass reaction vessel on a CEM Discover 908010 microwave reactor with IR-monitored temperature control.

Crude propargylated peptide (0.0166 mmol, 3 eq) was dissolved in a degassed aqueous solution of 6 M GnHCl/0.2 M Na₂HPO₄ (5.533 mL) containing TCEP hydrochloride (885.6 μL of 0.5 M aqueous solution, pH=7, 80 eq) and CuSO₄.5H₂O (885.6 μL of 0.5 M aqueous solution, 80 eq). After 30 min of incubation at 55° C., 2-azidoethylphosphonic acid (0.0996 mmol, 18 eq) was added and the reaction carried out under argon with microwave irradiation (20 W, 60° C., 2 h). The reaction mixture was acidified to pH=1 with conc. HCl, purified by solid phase extraction and lyophilised. Purification by by solid phase extraction using Alltech C18-LP 900 mg bed cartridges, then semi-preparative RP-HPLC, as described above for peptide 111, and lyophilisation yielded the purified phosphopeptide.

### Peptide 304

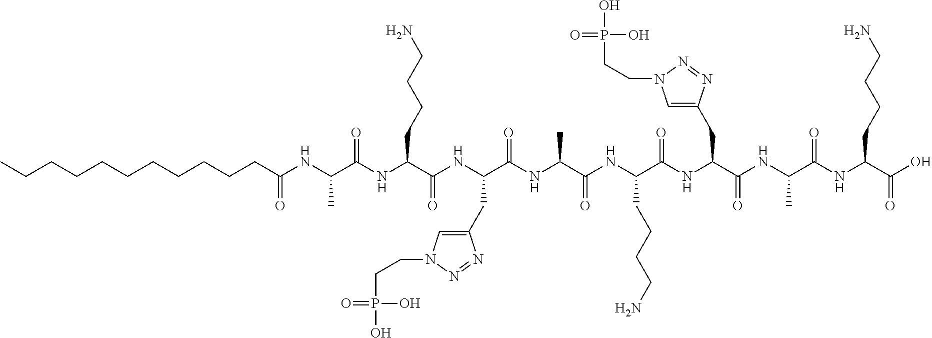 [SMILES: CCCCCCCCCCCC(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)O]

Purification by semi-preparative RP-HPLC using a linear gradient of 0% to 50% B over 20 min instead of 0% to 51% B over 33 minutes and lyophilisation yielded the purified peptide (0.63 mg, 3%) as a white solid in ca. 99% purity according to analytical HPLC. Rₜ 15.8 min (1-60% B over 20 min, 1 mL/min); m/z (ESI-MS): [M+2H⁺] calculated mass=646.7, observed mass=646.2; [M+3H⁺] calculated mass=431.5, observed mass=431.5.

### Peptide 305

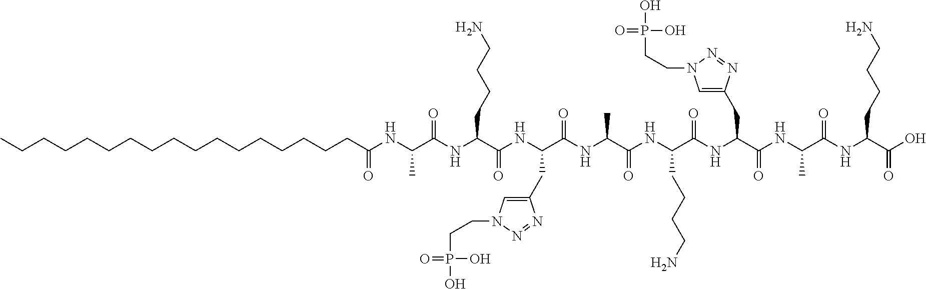 [SMILES: CCCCCCCCCCCCCCCCCC(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)N[C@@H](Cc1cn(CCP(=O)(O)O)nn1)C(=O)N[C@@H](C)C(=O)N[C@@H](CCCCN)C(=O)O]

Purification by semi-preparative RP-HPLC using a linear gradient of 0% to 70% B over 25 min instead of 0% to 51% B over 33 minutes and lyophilisation yielded the purified peptide (2.59 mg, 11%) as a white solid in ca. 99% purity according to analytical HPLC. Rₜ 21.0 min (1-75% B over 25 min, 1 mL/min); m/z (ESI-MS): [M+2H⁺] calculated mass=687.8, observed mass=688.0; [M+3H⁺] calculated mass=458.9, observed mass=459.2.

### Peptide 306

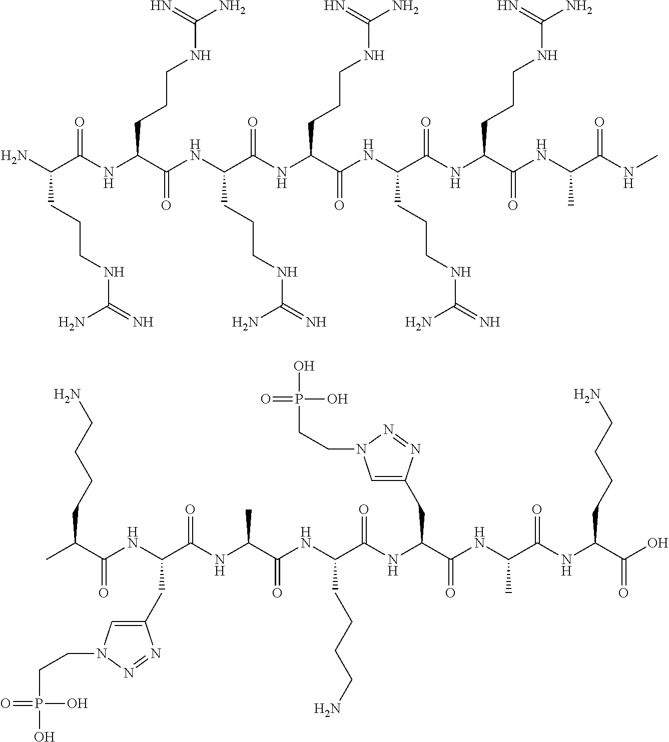

Purification by semi-preparative RP-HPLC using a linear gradient of 0% to 50% B over 20 min instead of 0% to 51% B over 33 minutes and lyophilisation yielded the purified peptide (3.1 mg, 9%) as a white solid in ca. 97% purity according to analytical HPLC using a Phenomenex Gemini C₁₈, 3 μM, 4.6 mm×150 mm column instead of a Waters XTerra MS C₁₈, 5 μm, 4.6 mm×150 mm column. Rₜ 10.7 min (0-50% B over 20 min, 1 mL/min); m/z (ESI-MS): [M+2H⁺] calculated mass=1023.6, observed mass=1023.0; [M+3H⁺] calculated mass=682.8, observed mass=682.4.

### Preparation of Metal Nanoparticles

### Preparation of iron oxide nanoparticles by coprecipitation

Iron nanoparticles were synthesised by coprecipitation in the presence of phosphopeptide 107. The results are provided below in Table 1.

In a typical experiment, analogue 107 was weighed into a 0.2 mL vial, a solution of 0.7 mol.L⁻¹NH₃.H₂O (31.5 mL, 22 mmol) was added and the mixture stirred until dissolution was complete. A drop of aqueous 1 mol.L⁻¹FeCl₃ (2.5 mL, 2.5 mmol) and a drop of 1 mol.L⁻¹ FeSO₄ in 1M HCl (1.25 mL, 1.25 mmol) were deposited on the walls of the vial. The vial was then flushed with N₂, fixed to a vortex shaker and stirred at 1800 rpm to mix the iron salt solutions with the ammonia solution. A black precipitate quickly appeared. After 30 min, the nanoparticles were separated from the supernatant liquor by centrifugation, washed twice with distilled water (under nitrogen atmosphere), freeze-dried and stored under nitrogen atmosphere.

TABLE 1
Synthesis of iron oxide nanoparticles.

| | | | | | | Stirring | |
|---|---|---|---|---|---|---|---|
| | FeCl₃ | FeSO₄ | NH₃ | 107 | Ratio of 107 to | speed | |
| Entry | (μmol) | (μmol) | (μmol) | (μmol) | metal | (rpm) | Particle size |
| a. | 2.5 | 1.25 | 44 | — | — | 1800 | 9.9 ± 2.8 nm |
| b. | 2.5 | 1.25 | 44 | — | — | 1800 | 11.9 ± 3.3 nm |
| c. | 2.5 | 1.25 | 44 | — | — | 2200 | 9.6 ± 2.6 nm |
| d. | 2.5 | 1.25 | 31 | — | — | 1800 | 8.3 ± 3.3 nm |
| e. | 2.5 | 1.25 | 44 | 0.188 | 0.05:1 | 1800 | 5.1 ± 3.0 nm |
| f. | 2.5 | 1.25 | 44 | 0.375 | 0.1:1 | 1800 | 4.6 ± 1.3 nm |
| g. | 2.5 | 1.25 | 44 | 0.75 | 0.2:1 | 1800 | 4.6 ± 0.8 nm |
| h. | 2.5 | 1.25 | 44 | 1.88 | 0.5:1 | 1800 | no precipitate |
| i. | 2.5 | 1.25 | 44 | 3.75 | 1:1 | 1800 | no precipitate |
| j. | 2.5 | 1.25 | 44 | 7.5 | 2:1 | 1800 | no precipitate |

The particles prepared in the presence of phosphopeptide 107 were significantly more stable in suspension and therefore more difficult to separate by centrifugation, than the particles prepared in the absence of additives, which were easily separated by centrifugation.

The particles prepared in the presence of phosphopeptide 107 showed signs of oxidation after storage overnight, indicated by a change in the colour of the black powder initially obtained to a red-brown powder. In contrast, the particles prepared in the absence of phosphopeptides remained black for several weeks.

Accurate determination of the size of the particles over a large population was not possible, due to the highly aggregated nature of the particles after evaporation of a droplet of a suspension of the particles on the TEM grid. The particle sizes reported above correspond to estimations of the average particle sizes, based on the sizes of particles on the edges of the aggregates as determined using TEM.

Control experiments (Table 1, Entries a-d) were carried out to confirm the reproducibility of the synthesis and its sensitivity to small changes in the experimental protocol. The experimental procedures for Entries a and b were identical and resulted in very similar iron oxide nanoparticles with particle sizes of 10 nm (FIGS. 17 and 18) and 12 nm, respectively. The particles displayed a tendency to aggregate and generally showed spheroid morphology. The product nanoparticles were obtained in the form of a black powder. Electron diffraction patterns showed five diffuse rings that can be indexed to Fe₃O₄ (220), Fe₃O₄ (311), Fe₃O₄ (400), Fe₃O₄ (511) and Fe₃O₄ (440). EDS measurements confirmed the presence of iron and oxygen.

Modification of the experimental procedure by varying parameters such as the stirring speed (Table 1, Entry c) or the concentration of ammonia in solution (Table 1, Entry d) did not significantly affect the size, composition, or morphology of the nanoparticles.

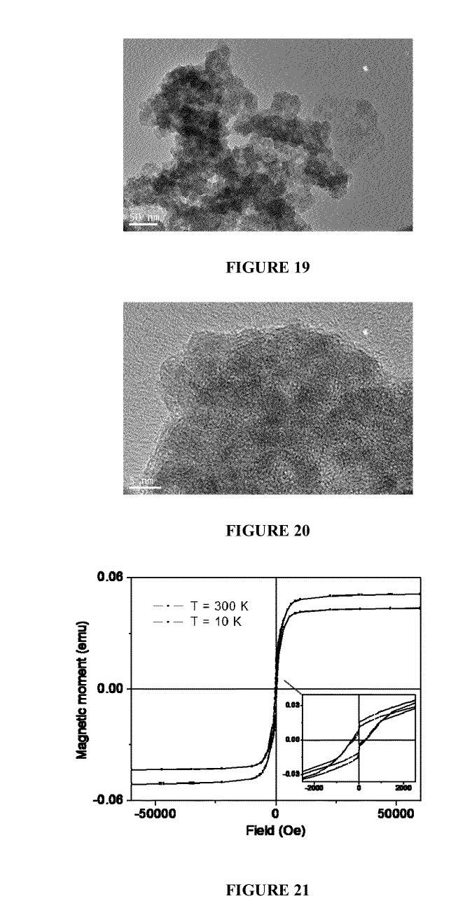

The introduction of low ratios of phosphopeptide 107 (Table 1, Entries e-g) as an additive resulted in a significant reduction in the size of the product nanoparticles (FIGS. 19 and 20). The average diameter of the iron oxide nanoparticles was reduced from about 10 nm to about 5 nm. In contrast to the nanoparticles obtained in the control experiments, the nanoparticles obtained using phosphopeptide 107 were obtained in the form of a powder that was initially black, but changed to red-brown overnight. Electron diffraction patterns for these samples showed a dramatic decrease in the crystallinity of the sample and therefore the diffuse rings could not be precisely attributed. EDS measurements confirmed the presence of iron. The particles prepared in the presence of different ratios of phosphopeptide 107 to iron (Table 1, Entries e-g) had relatively similar shape and similar size (within the variance calculated).

The introduction of high ratios of phosphopeptide 107 (Table 1, Entries h-j) as an additive resulted in no precipitation. Instead, the colour of the reaction mixture slowly changed from yellow to orange over about 5 minutes.

### Preparation of Metal Nanoparticles by Reduction

### Iron Nanoparticles

### Using Phosphopeptides 107-111

Iron nanoparticles were prepared by reduction in the presence of 107-111.

FeSO₄.7H₂O (1.98 mg, 7 mmol) was dissolved in 2 mL of previously degassed deionised water. Trisodium citrate (0.18 mg, 0.7 mmol) was added for Entry b (Table 2), phosphopeptide 107-111 and peptide 77 (0.35 mmol) were added for Entries c-h respectively, and 3-O-(phospho)-serine 112 (0.26 mg, 1.4 mmol) was added for Entry i. No additive was used for Entry a. The mixtures were stirred until dissolution was complete. NaBH₄ (0.5 mg, 13 mmol) in 1 mL of deionised water was then added and the mixture stirred vigorously under an atmosphere of nitrogen. A black precipitate quickly appeared and the mixture was further stirred for 10 min. The particles were magnetically decanted, washed three times with ethanol, dried, and stored under nitrogen atmosphere.

The nanoparticles size (and shell thickness, where appropriate) are provided below in Table 2.

TABLE 2
Iron-iron oxide core-shell nanoparticles size and shell thickness.

| | | | Shell thickness |
|---|---|---|---|
| Entry | Additive | Particle size (nm) | (nm) |
| a. | — | 58 ± 13 | 3.2 ± 1 |
| b. | trisodium citrate | 67 ± 22 | 3.6 ± 0.9 |
| c. | 107 | 20 ± 5 | 3.9 ± 0.8 |
| e | 108 | 19 ± 4 | 3.3 ± 0.5 |
| e. | 109 | 19 ± 4 | 5.0 ± 1.6 |
| f | 110 | 18 ± 5 | 3.5 ± 0.9 |
| g. | 111 | 8.8 ± 2 | — |
| h. | 77 | 49 ± 7 | 4.0 ± 1.0 |
| i. | 112 | 29 ± 8 | 4.4 ± 1.4 |

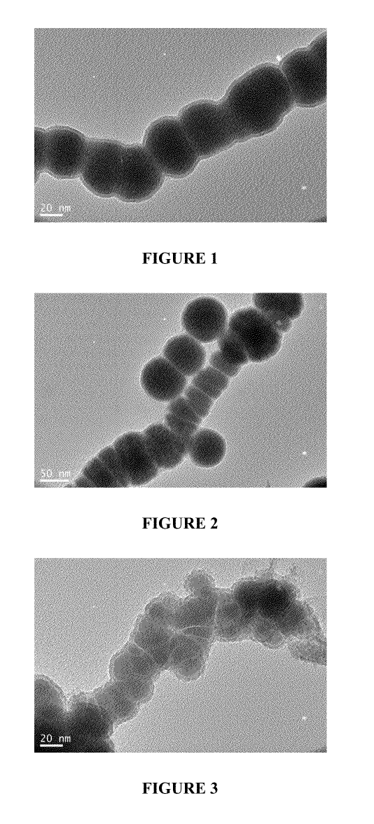

Reduction of iron (II) sulfate in water using NaBH₄ in the absence of any additives (Table 2, Entry a) produced iron-iron oxide core-shell nanoparticles with an overall size of 58±13 nm and a shell thickness of 3.2±1 nm (FIG. 1). The particles were attached to each other in the form of chain-like structures that extended beyond a few hundred nanometres. Due to their large size, the structures were not stable when suspended in solution. The structures had a strong tendency to aggregate and precipitated out of the solution after a few seconds.

Repeating the reduction in the presence of trisodium citrate (10:1 metal to citrate) produced particles with a size and morphology very similar to those produced in Entry a (FIG. 2). The electron diffraction pattern showed two diffuse rings that can be indexed to Fe (110) and Fe (211). EDS measurements confirmed the presence of iron and oxygen in the samples. The iron nanostructures prepared in the presence of sodium citrate displayed weaker tendency to agglomerate and remained stable in suspension for a longer period of time, than those prepared in the absence of any additives.

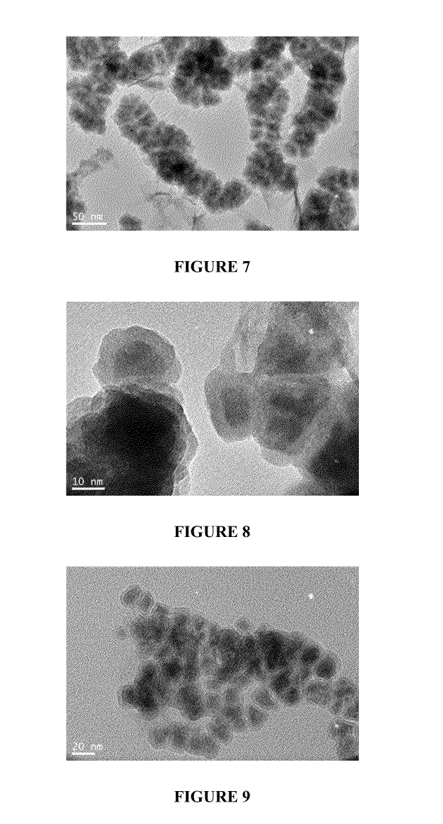

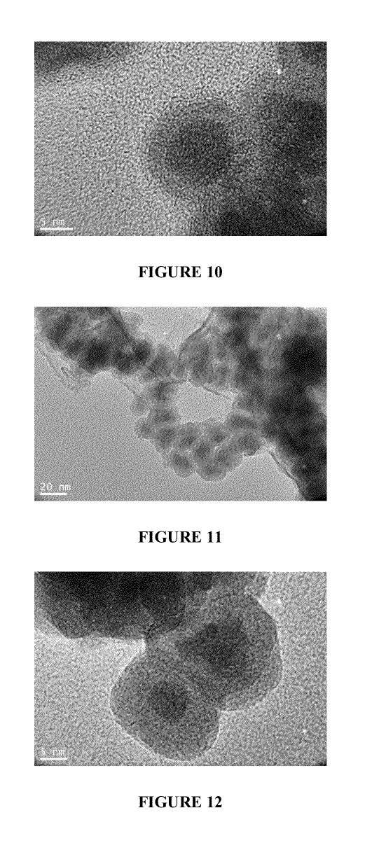

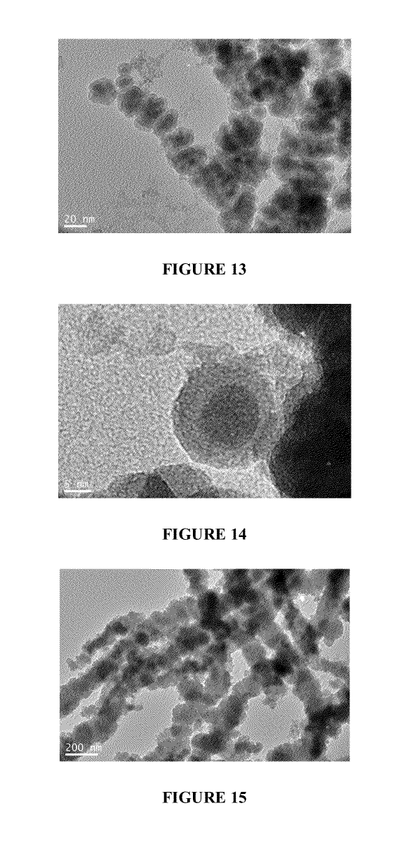

The use of phosphopeptides 107-110 (Table 2, Entries c-f) as additives in the synthesis of iron nanoparticles dramatically reduced the particles size from about 60 nm to about 20 nm. The particles prepared in the presence of these analogues were all similar in shape and size (within the variance calculated). These iron-iron oxide core-shell nanoparticles were also joined together as chain-like structures, but with nanoparticles of smaller diameter than the structures formed in Entries a and b (FIGS. 7-14). However, in contrast to the structures formed in Entries a and b, the structures formed in the presence of phosphopeptides 107-110 formed stable suspensions when dispersed in ethanol. Electron diffraction patterns for these samples showed four diffuse diffraction rings that can be indexed to Fe₃O₄ (311), Fe (110), Fe (200) and Fe (211). EDS measurements confirmed the presence of iron and oxygen.

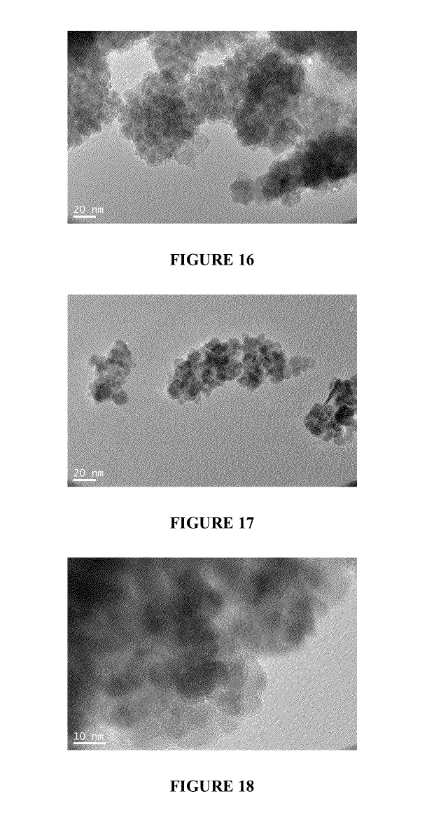

The use of phosphopeptide 111 as additive reduced the size of the resulting nanoparticles more significantly, resulting in highly aggregated nanoparticles with an average diameter of 8.8±2 nm (FIGS. 15 and 16). The particles were not core-shell nanoparticles. In contrast to the particles obtained in the control experiments, which were obtained in the form of black powders that remained black for several days, the particles prepared in presence of phosphopeptide 111 were obtained in the form of a powder that was initially black, but changed to grey overnight. The electron diffraction pattern showed two diffuse rings that can be indexed to either Fe₃O₄ (311) and Fe₃O₄ (440) or to γ-Fe₂O₃ (311) and γ-Fe₂O₃ (440). EDS confirmed the presence of iron and oxygen.

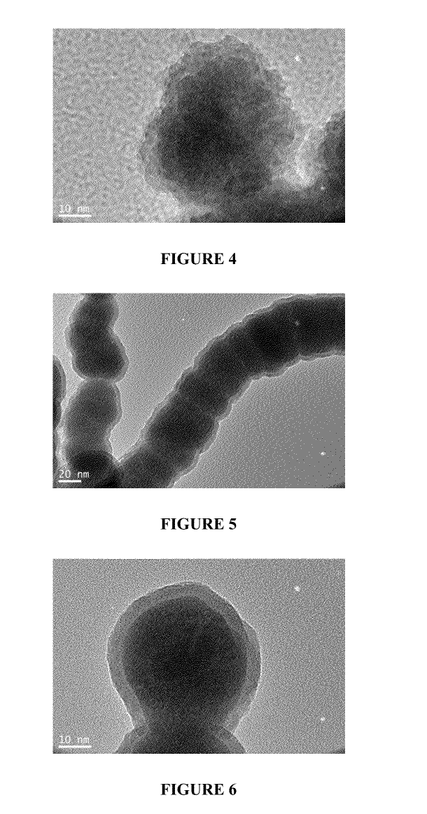

Control experiments were also carried out with peptide 77 (Table 2, Entry h) or 3-O-(phospho)-serine 112 (Table 2, Entry i). Carrying out the experimental procedure using peptide 77 as the additive gave rise to large iron-iron oxide core-shell nanoparticles with a diameter of 49±7 nm (FIGS. 5 and 6), while the use of 3-O-(phospho)-serine 112 led to the formation of irregularly shaped iron-iron oxide core-shell nanoparticles with an average diameter of 29±8 nm (FIGS. 3 and 4). These nanoparticles also formed chain-like structures. The electron diffraction patterns for these nanoparticles showed three diffuse rings that can be indexed to Fe₃O₄ (311), Fe (110) and Fe (211). EDS confirmed the presence of iron and oxygen.

For Entries a-g, h, and i (Table 2), the thickness of the iron oxide shells was measured to be about 4 nm.

Diffuse diffraction rings shown by the electron diffraction indicated that all of the samples had low crystallinity. This was confirmed by HR-TEM of the samples, where no lattice fringes were observed for any of the samples, highlighting the absence of long range ordering of the iron atoms within each nanoparticle.

The magnetic properties of the nanoparticles obtained in the presence of trisodium citrate and in the presence of phosphopeptide 107 (Table 2, Entries b and c) were evaluated. M(H) loop measurements were obtained between ±60 kOe at both T=10 K and T=300 K. The masses of sample used were very small therefore the magnetic moment observed could not be related to the magnetization in emu.g⁻¹.

The M(H) plot for the iron-iron oxide core-shell nanoparticles grown in presence of trisodium citrate (FIG. 21) shows a clear ferromagnetic behaviour of the particles with the observation of a magnetic hysteresis at both 10 K and 300 K. At low fields, sudden steps in the magnetic moment were observed with the magnetisation suddenly dropping when decreasing field approaches 0 Oe and the magnetisation suddenly increasing when increasing field approaches 0 Oe. The size of the steps decreased when the sample was measured at 300 K.

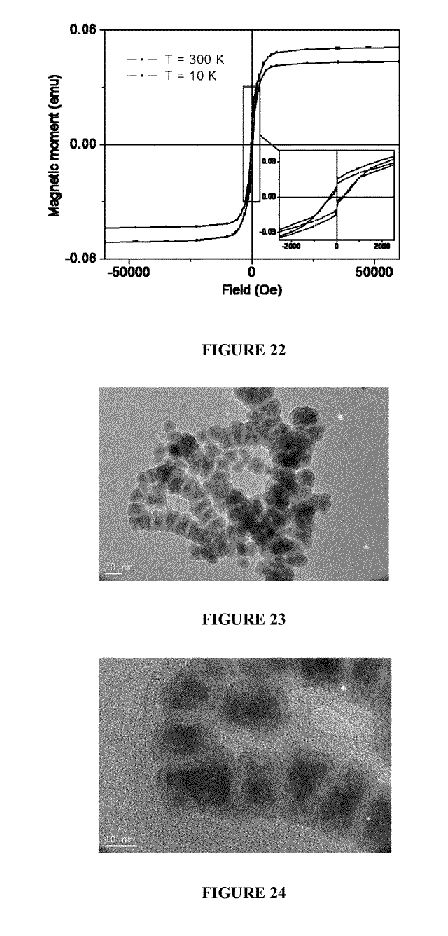

The M(H) plot for the iron nanoparticles grown in the presence of analogue 107 also shows a clear ferromagnetic behaviour of the particles with observation of a magnetic hysteresis at both 10 K and 300 K (FIG. 22). Steps in the magnetic moment around low field (similar to the ones observed in FIG. 21) can be clearly observed at T=10 K. Again, the size of these steps decreases with increasing temperature, until they disappear at T=300 K. These steps affected the value of the sample coercivity and remanent magnetization as a function of temperature, inducing a dramatic increase in remanent magnetization at low temperature. A small shift of the magnetic hysteresis loop in the direction of the applied field was also observed at low temperature, but disappeared above 25 K. The amplitude of this shift was similar whether the system was cooled at zero fields or with an applied field of 6 T, the latter being known to sometimes increase the exchange bias phenomenon. The saturation moment decreases with increasing temperature.

### Using Phosphopeptides 207, 209, 211, and 303-306

Iron nanoparticles were also prepared by reduction in the presence of phosphopeptides 207, 209, 211, and 303-306.

FeSO₄.7H₂O (1.95 mg, 7 mmol) was dissolved in 1-1.5 mL of previously degassed distilled water and the phosphopeptide (0.35 mmol) was dissolved in 1 mL of previously degassed distilled water. The solutions were mixed and stirred under nitrogen for 15 min. Then the NaBH₄ solution (40 mmol, 20 μL of a 2M solution in triethylene glycol dimethyl ether) was added all at once and the reaction solution stirred vigorously under nitrogen atmosphere (stirring speed 1000 rpm). A black precipitate immediately appeared and the mixture was stirred for 10 min. The reaction mixture was sonicated in order to separate all particles from the stirrer bar, centrifuged and the supernatant solution was decanted. The black precipitate was suspended in degassed ethanol, sonicated for 5 min, centrifuged and the supernatant decanted (2×) and then the particles were dried in vacuo.

The particles formed in the presence of phosphopeptides 207, 209, and 211 were iron-iron oxide core-shell nanoparticles. The particles size and shell thickness are provided below in Table 3.

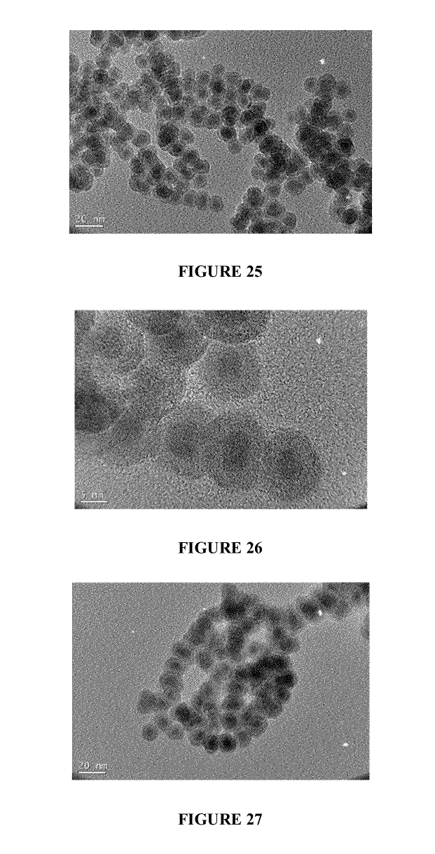

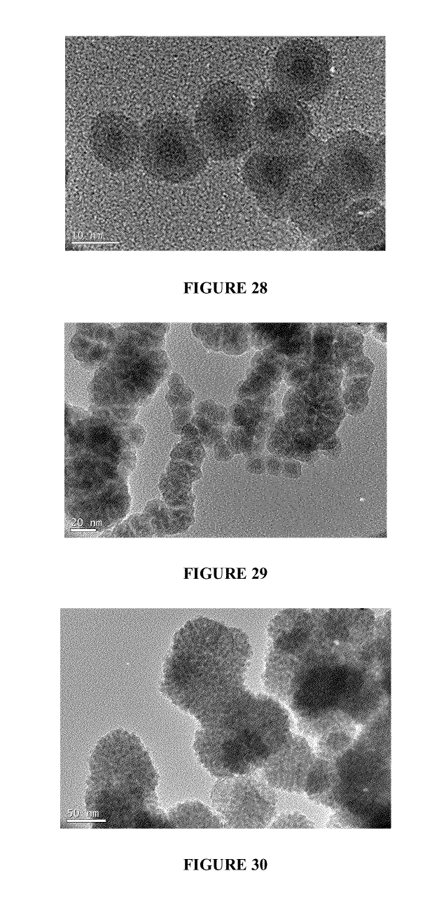

The particles were spheroidal in shape and aggregated in chain-like structures (FIGS. 23-28). The monodispersity was good.

The nanoparticles formed stable suspensions, for example a suspension of the iron nanoparticles formed using 207 was stable overnight.

Electron diffraction patterns for the particles showed four diffuse diffraction rings that can be indexed to Fe₃O₄ (311), Fe (110), Fe (200) and Fe (211). EDS measurements confirmed the presence of iron and oxygen.

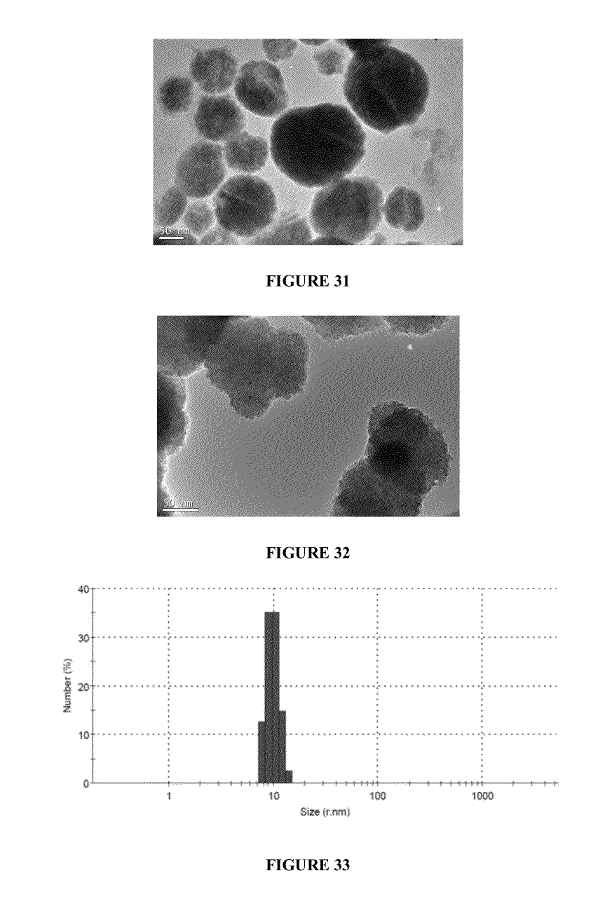

The particles formed in the presence of 303-306 are shown in FIGS. 29-32. The particle sizes and shell thicknesses of the particles are provided below in Table 3. The particles formed in the presence of phosphopeptide 304 were highly aggregated and the electron diffraction pattern is very faint.

A control experiment without phosphopeptide was also carried out. Iron-iron oxide core-shell nanoparticles were formed. The particle size and shell thickness of the particles is provided in Table 3. The particles displayed a high degree of polydispersity. The electron diffraction pattern showed three diffuse rings which can be indexed to Fe (110), Fe (200) and Fe (211). EDS measurements confirmed that the sample had high iron and oxygen content. The particles have a strong tendency to aggregate and precipitate out of the suspension in ethanol after only a couple of minutes.

TABLE 3
Iron-iron oxide core-shell nanoparticles size and shell thickness.

| Phosphopeptide | Particle size (nm) | Shell thickness (nm) |
|---|---|---|
| 207 | 15.7 ± 2.8 | 2.9 ± 0.6 |
| 209 | 12.0 ± 1.3 | 3.1 ± 0.3 |
| 211 | 13.8 ± 1.6 | 3.5 ± 0.5 |
| 303 | 21.7 ± 12.1 | 3.1 ± 0.3 |
| 304 | 87.0 ± 14.0 | — |
| 305 | 145.4 ± 29.5 | 3.2 ± 0.3 |
| 306 | 113.7 ± 12.5 | — |
| — | 76.9 ± 20 | 2.9 ± 0.68 |

Dynamic light scattering experiments were carried out on nanoparticles prepared in the presence of 209. 1 mL of the crude reaction mix was diluted with 3 mL of NaHCO₃/Na₂CO₃ buffer (pH=10) and 0.1% (v/v) Triton X-100 (reduced form) was added to break up the aggregates. Then the sample was sonicated at 45° C. for 20 min. The theoretical material refractive index of magnetite (2.42) and dispersant viscosity, refractive index and dielectric constant of pure water were used to characterise the sample. The measurement was carried out at 65° C. and revealed an average particle size of 10.0 nm (FIG. 33).

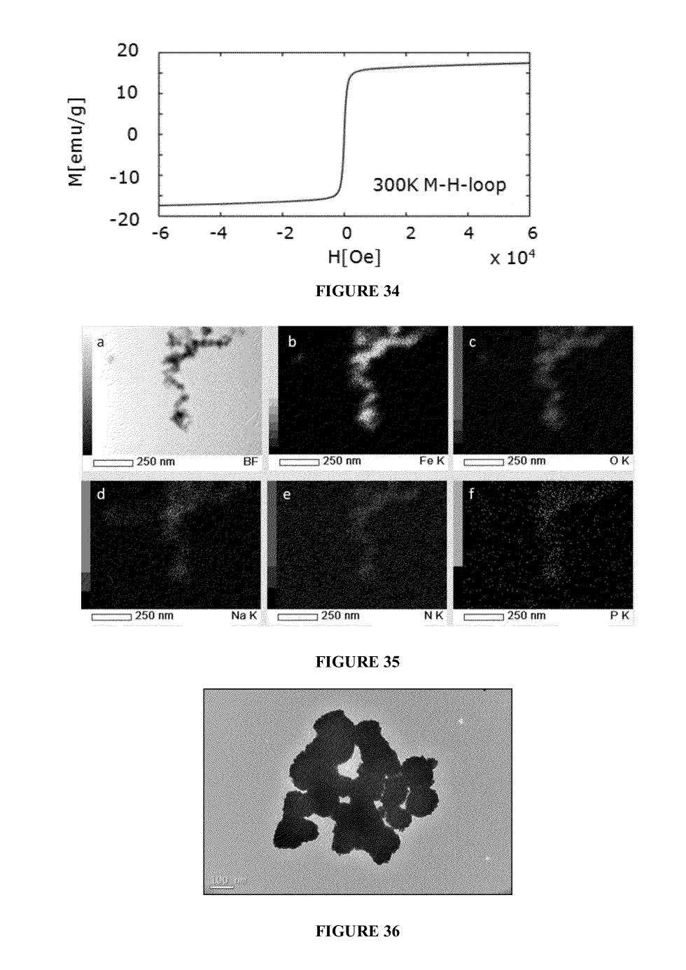

The magnetic properties of nanoparticles prepared in the presence of 209 were evaluated by measuring the magnetic hysteresis loop at 300 K from −6 to 6 Tesla. The magnetisation curve intercepts at the origin, indicating an absence of remnant magnetisation (M_{R}) and coercivity (H_{C}) (FIG. 34). The nanoparticles exhibit superparamagnetic behaviour.

Quantitative chemical analyses of nanoparticles prepared in the presence of 209 were collected using an X-ray energy dispersive spectroscopy attachment in the STEM mode (scanning TEM) at an electron beam accelerating voltage of 200 kV. FIG. 35 shows the bright field image of the scanned area (a) and the elemental maps recorded for Fe (b), 0 (c), Na (d), N (e) and P (f). The measurement confirms that the high iron content seen in standard

EDS analysis is located in the area of the depicted nanoparticles in the STEM image. The abundant presence of oxygen atoms stems from the iron oxide shell of the core/shell particles as well as phosphopeptide 209, which is still on the surface of the particles. This is further confirmed by a nitrogen content of circa 10% and a slight enrichment of phosphorous in the analysed area. The sodium atoms were introduced during the synthesis of the nanoparticles using NaBH₄ as reducing agent and are possibly bound to oxide anions of the iron oxide shell or carboxylate anions of phosphopeptide 209.

### Platinum Nanoparticles

Platinum nanoparticles were prepared by reduction in the presence of phosphopeptide 209.

Pt(NH₃)₄(NO₃)₂ (1.36 mg, 3.5 mmol) was dissolved in 1 mL of previously degassed distilled water and the phosphopeptide (0.33 mg. 0.175 mmol) was dissolved in 1 mL of previously degassed distilled water. The solutions were mixed and stirred under nitrogen for 15 min (colourless solution). Then the NaBH₄ solution (35 mmol, 17.5 μL of a 2M solution in triethylene glycol dimethyl ether) was added all at once and the reaction solution stirred vigorously under nitrogen atmosphere (stirring speed 1000 rpm) for 2 h. The reaction mixture turned dark during that time. The mixture was sonicated, centrifuged and the supernatant solution was decanted. The black precipitate was suspended in degassed ethanol (500 μL), sonicated for 5 min, centrifuged and the supernatant decanted (2×) and then the particles were dried in vacuo.

A control experiment without 209 was also carried out.

Pt(NH₃)₄(NO₃)₂ (3.5 mmol) dissolved in 2 mL of degassed distilled water, then 17.5 μl 2M NaBH₄ solution in triglyme (35 mmol) added. The reaction mixture was stirred for 15 min under nitrogen, then sonicated, centrifuged and the supernatant solution decanted. The precipitate was suspended in degassed ethanol (500 μL), sonicated for 5 min, centrifuged and the supernatant decanted (2×) and then the particles were dried in vacuo.

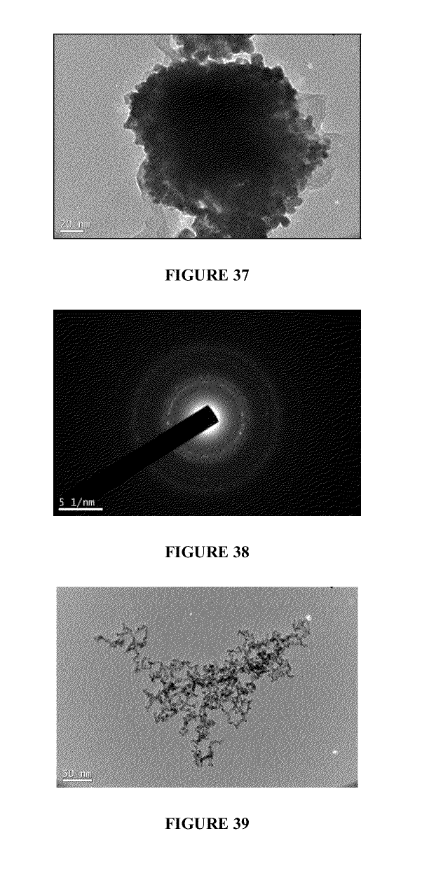

Reduction in the presence of 209 provided large, very polydisperse aggregates with a diameter of 163±63 nm (FIGS. 36 and 37). The electron diffraction pattern of the nanoparticles is shown in FIG. 38. EDS confirmed that the nanoparticles have high Pt content.

### Palladium Nanoparticles

Palladium nanoparticles were prepared by reduction in the presence of phosphopeptide 209.

PdCl₂ (0.62 mg, 3.5 mmol) was dissolved in 1 mL of previously degassed distilled water and the phosphopeptide (0.33 mg. 0.175 mmol) was dissolved in 1 mL of previously degassed distilled water. The solutions were mixed and stirred under nitrogen for 15 min (brown solution). Then the NaBH₄ solution (35 mmol, 17.5 μL of a 2M solution in triethylene glycol dimethyl ether) was added all at once and the reaction solution stirred vigorously under nitrogen atmosphere (stirring speed 1000 rpm) for 15 min. The reaction mixture turned black immediately upon addition of the reducing agent. The mixture was sonicated, centrifuged and the supernatant solution was decanted. The black precipitate was suspended in degassed ethanol (500 μL), sonicated for 5 min, centrifuged and the supernatant decanted (2×) and then the particles were dried in vacuo.

A control experiment without 209 was also carried out using a procedure analogous to that described above for the platinum nanoparticles, using PdCl₂ instead of Pt(NH₃)₄(NO₃)₂.

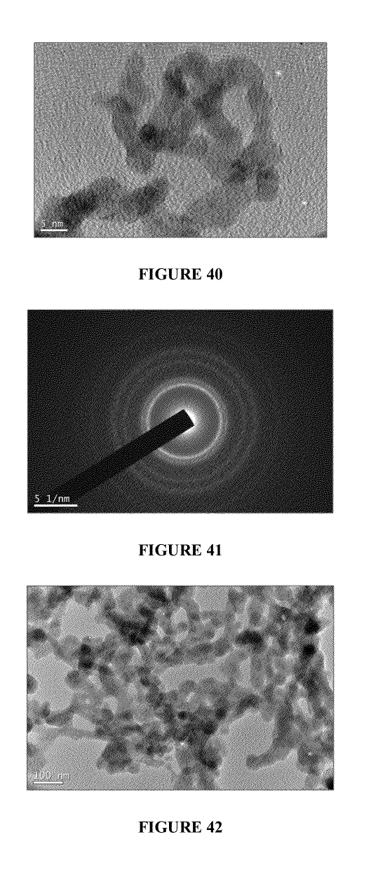

Reduction in the presence of 209 provided nanowires having an average diameter of 5.0±1.1 nm (FIGS. 39 and 40). Many crystal fringes visible by HRTEM. The electron diffraction pattern of the nanoparticles is shown in FIG. 41. EDS confirmed that the nanoparticles have high Pd content.

### Ruthenium Nanoparticles

Ruthenium nanoparticles were prepared by reduction in the presence of phosphopeptide 209.

RuCl₃.xH₂O (0.73 mg, 3.5 mmol) was dissolved in 1 mL of previously degassed distilled water and the phosphopeptide (0.33 mg. 0.175 mmol) was dissolved in 1 mL of previously degassed distilled water. The solutions were mixed and stirred under nitrogen for 15 min (dark brown solution). Then the NaBH₄ solution (35 mmol, 17.5 μL of a 2M solution in triethylene glycol dimethyl ether) was added all at once and the reaction solution turned yellow, blue and then black within a couple of seconds. After stirring vigorously under nitrogen atmosphere (stirring speed 1000 rpm) for 15 min, the reaction mixture was sonicated, centrifuged and the supernatant solution was decanted. The black precipitate was suspended in degassed ethanol (500 μL), sonicated for 5 min, centrifuged and the supernatant decanted (2×) and then the particles were dried in vacuo.

A control experiment without 209 was also carried out using a procedure analogous to that described above for the platinum nanoparticles, using RuCl₃.xH₂O instead of Pt(NH₃)₄(NO₃)₂.

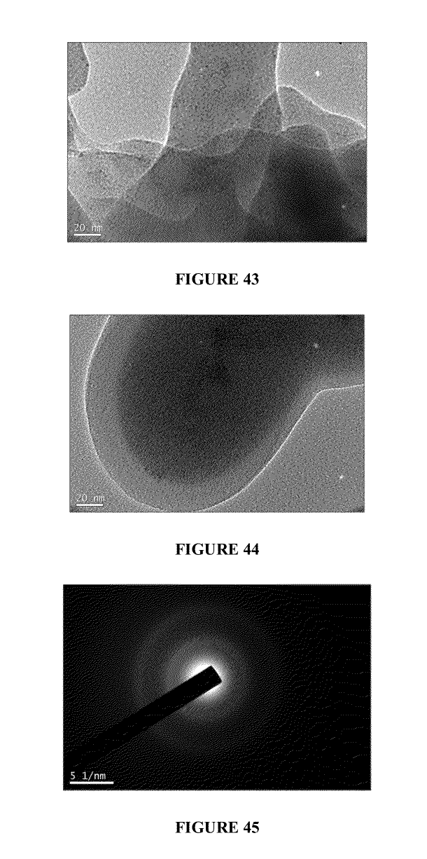

Reduction in the presence of 209 provided large and polydisperse aggregated wires/sheets with an average diameter of 63±37 nm, which extend over several micrometer nanowires (FIGS. 42, 43, and 44). No crystal fringes are visible. The nanoparticles may be ruthenium-ruthenium oxide core-shell nanoparticles. The electron diffraction pattern shows very faint rings (FIG. 45). EDS confirmed that the nanoparticles have high Ru content.

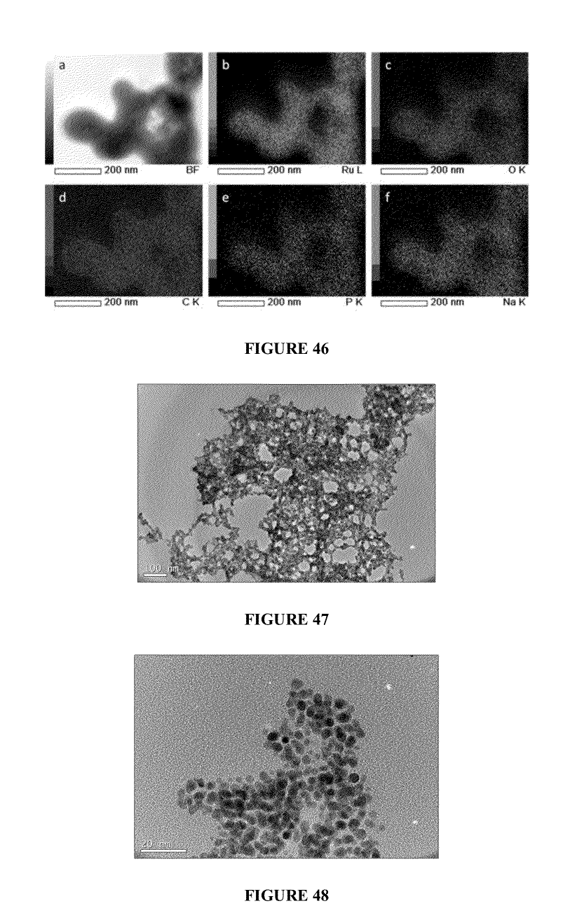

Quantitative chemical analyses of the nanoparticles were collected using an X-ray energy dispersive spectroscopy (EDS) attachment in the STEM mode (scanning TEM) at an electron beam accelerating voltage of 200 kV. FIG. 46 shows the bright field image of the scanned area (a) and the elemental maps recorded for Ru (b), 0 (c), C (d), P (e) and Na (f). The measurement confirms that the high ruthenium content seen in standard EDS analysis is located in the area of the depicted nanoparticles in the STEM image. The abundant presence of oxygen atoms may stem from the ruthenium oxide shell of ruthenium-ruthenium oxide core/shell particles as well as the phosphopeptide, which is still on the surface of the particles. This is further confirmed by a high carbon and phosphorus content. The sodium atoms were introduced during the synthesis of the nanoparticles using NaBH₄ as reducing agent and are possibly bound to oxide anions of the ruthenium oxide shell or carboxylate anions of the phosphopeptide.

### Silver Nanoparticles

Silver nanoparticles were prepared by reduction in the presence of phosphopeptide 209.

Silver trifluoroacetate (0.77 mg, 3.5 μmol) was dissolved in 1 mL of previously degassed distilled water and the phosphopeptide (0.33 mg. 0.175 μmol) was dissolved in 1 mL of previously degassed distilled water. The solutions were mixed and stirred under nitrogen for 15 min (colourless solution). Then the NaBH₄ solution (35 mmol, 17.5 μL of a 2M solution in triethylene glycol dimethyl ether) was added all at once and the reaction solution stirred vigorously under nitrogen atmosphere (stirring speed 1000 rpm) for 15 min. The brown reaction mixture was centrifuged and the supernatant solution decanted. The brown precipitate was suspended in degassed ethanol (500 mL), sonicated for 5 min, centrifuged and the supernatant decanted (2×) and then the brown-red particles were dried in vacuo.

A control experiment without 209 was also carried out using a procedure analogous to that described above for the platinum nanoparticles, using silver trifluoroacetate instead of Pt(NH₃)₄(NO₃)₂.

The suspension obtained in the control experiment was not stable in EtOH after purification. In contrast, the nanoparticles prepared in the presence of 209 were stable suspension for at least 8 days.

### Rhodium Nanoparticles

Rhodium nanoparticles were prepared by reduction in the presence of phosphopeptide 209.

RhCl₃.xH₂O (0.73 mg, 3.5 mmol) was dissolved in 1 mL of previously degassed distilled water (orange solution) and the phosphopeptide (0.33 mg. 0.175 mmol) was dissolved in 1 mL of previously degassed distilled water. The solutions were mixed and stirred under nitrogen for 15 min (yellow solution). Then the NaBH₄ solution (35 mmol, 17.5 μL of a 2M solution in triethylene glycol dimethyl ether) was added all at once and the reaction solution turned dark (black-brown) immediately. The mixture was stirred vigorously under nitrogen atmosphere (stirring speed 1000 rpm) for 15 min. The reaction mixture was centrifuged and the supernatant solution decanted. The black precipitate was suspended in degassed ethanol (500 mL), sonicated for 5 min, centrifuged and the supernatant decanted (2×) and then the particles were dried in vacuo.

A control experiment without 209 was also carried out using a procedure analogous to that described above for the platinum nanoparticles, using RhCl₃.xH₂O instead of Pt(NH₃)₄(NO₃)₂.

The crude rhodium reaction suspension in the control experiment was not stable. Nanoparticles precipitated from the reaction mixture after about 1 hour. In contrast, nanoparticles prepared using 209 were stable in suspension for at least 7 days.

### Gold Nanoparticles

Gold nanoparticles were prepared by reduction in the presence of phosphopeptide 209.

AuCl₃ (1.06 mg, 3.5 mmol) was dissolved in 1 mL of previously degassed distilled water and the phosphopeptide (0.33 mg. 0.175 mmol) was dissolved in 1 mL of previously degassed distilled water. The solutions were mixed and stirred under nitrogen for 15 min (light yellow solution). Then the NaBH₄ solution (35 mmol, 17.5 μL of a 2M solution in triethylene glycol dimethyl ether) was added all at once and the reaction solution turned purple-black immediately. The mixture was stirred vigorously under nitrogen atmosphere (stirring speed 1000 rpm) for 15 min. The reaction mixture was centrifuged and the supernatant solution decanted. The dark purple precipitate was suspended in degassed ethanol (500 μL), sonicated for 5 min, centrifuged and the supernatant decanted (2×) and then the particles were dried in vacuo.

A control experiment without 209 was also carried out using a procedure analogous to that described above for the platinum nanoparticles, using AuCl₃ instead of Pt(NH₃)₄(NO₃)₂.

In the control experiment, the particles aggregated and precipitated during the reaction. In contrast, the nanoparticles prepared using 209 were stable in suspension for at least 7 days.

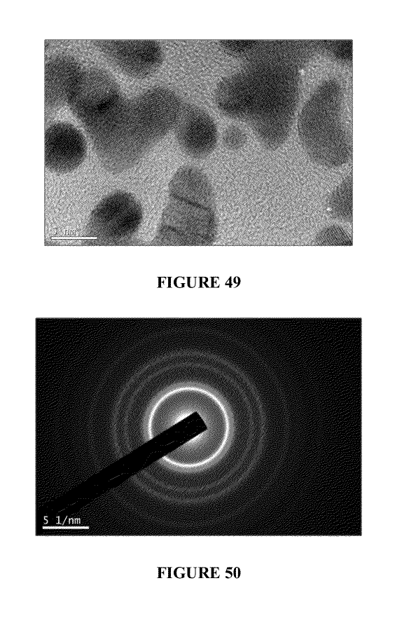

Reduction in the presence of 209 provided very small, relatively monodisperse nanoparticles with an average diameter of 4.4±0.7 nm (FIGS. 47, 48, and 49). The nanoparticles are very crystalline—many crystal fringes are visible. The electron diffraction pattern of the nanoparticles is shown in FIG. 50. EDS confirmed that the nanoparticles have high Au content. EDS also confirmed the presence of phosphorus, which is present in 209.

### Cobalt Nanoparticles

Cobalt nanoparticles were prepared by reduction in the presence of phosphopeptide 209.

CoCl₃. 6 H₂O (0.83 mg, 3.5 mmol) was dissolved in 1 mL of previously degassed distilled water and the phosphopeptide (0.33 mg. 0.175 mmol) was dissolved in 1 mL of previously degassed distilled water. The solutions were mixed and stirred under nitrogen for 15 min (colourless solution). Then the NaBH₄ solution (35 mmol, 17.5 μL of a 2M solution in triethylene glycol dimethyl ether) was added all at once and the reaction solution turned dark brown immediately. The mixture was stirred vigorously under nitrogen atmosphere (stirring speed 1000 rpm) for 15 min. The reaction mixture was sonicated, centrifuged and the supernatant solution decanted. The black precipitate was suspended in degassed ethanol (500 μL), sonicated for 5 min, centrifuged and the supernatant decanted (2×) and then the particles were dried in vacuo.

A control experiment without 209 was also carried out using a procedure analogous to that described above for the platinum nanoparticles, using CoCl₃. 6 H₂O instead of Pt(NH₃)₄(NO₃)₂.

The suspension obtained in the control experiment precipitated after 2 hours.

The suspension obtained by reduction in the presence of 209 began to precipitate after 4 hours, but the rate of preciptation was very slow. Precipitation was complete after 6 days.

### Nickel Nanoparticles

Nickel nanoparticles were prepared by reduction in the presence of phosphopeptide 209.

Ni(OAc)₂. 4 H₂O (0.87 mg, 3.5 μmol) was dissolved in 1 mL of previously degassed distilled water and the phosphopeptide (0.33 mg. 0.175 μmol) was dissolved in 1 mL of previously degassed distilled water. The solutions were mixed and stirred under nitrogen for 15 min (colourless solution). Then the NaBH₄ solution (35 μmol, 17.5 μL of a 2M solution in triethylene glycol dimethyl ether) was added all at once and the reaction solution turned dark brown immediately. It was stirred vigorously under nitrogen atmosphere (stirring speed 1000 rpm) for 15 min. The reaction solution was centrifuged at 14500 rpm for 10 min, but no precipitation occurred.

A control experiment without 209 was also carried out using a procedure analogous to that described above for the platinum nanoparticles, using Ni(OAc)₂. 4 H₂O instead of Pt(NH₃)₄(NO₃)₂.

Black particles aggregated and precipitated during the control experiment.

In contrast, reduction in the presence of 209 provided a brown solution with only precipitated and changed colour after 6 days.

Summary of Metal Nanoparticles Prepared Using Phosphopeptide 209

| Metal | Precursor | Particle size (nm) | Suspension stable? |
|---|---|---|---|
| Fe | FeSO₄•7 H₂O | 15.1 ± 1.6† | Yes |
| Pt | Pt(NH₃)₄(NO₃)₂ | 163 ± 63†† | Not determined |
| Pd | PdCl₂ | 5.0 ± 1.1 | Not determined |
| Ru | RuCl₃•x H₂O | 63 ± 37 | Not determined |
| Ag | Ag(CF₃COO) | Not determined | Yes |
| Ir | IrCl₃•x H₂O | — | (Solution/no visible |
| | | | precipitation) |
| Rh | RhCl₃•x H₂O | Not determined | Yes |
| Au | AuCl₃ | 4.4 ± 0.7 | Yes |
| Cu | Cu(OAc)₂ | — | (Solution/no visible |
| | | | precipitiation) |
| Co | CoCl₃•6 H₂O | Not determined | Moderate |
| Ni | Ni(OAc)₂•4 H₂O | Not determined* | (Solution/no visible |
| | | | precipitation)* |

†Core 12.0 ± 1.3, shell 3.1 ± 0.3.
††Aggregates of smaller particles.
*Precipitated and changed colour after 6 days.

### INDUSTRIAL APPLICATION

The metal nanoparticles and metal nanoparticle-phosphopeptide complexes of the present invention have numerous applications, as would be appreciated by a person skilled in the art. For example, the particles may be used in cancer treatment by hyperthermia, contrast enhancement in medical imaging, new drug delivery methods, and as catalysts.

Although the invention has been described by way of example and with reference to particular embodiments, it is to be understood that modifications and/or improvements may be made without departing from the scope of the invention.

1. A metal nanoparticle-phosphopeptide complex comprising:
a metal nanoparticle; and
a phosphopeptide comprising two or more contiguous peptide motifs and two or more phosphorus-containing groups capable of interacting with the surface of the metal nanoparticle,
wherein the amino acids at the equivalent position in each peptide motif have similar structural and/or electronic properties, and
wherein each phosphorus-containing group is bound to an amino acid in the two or more contiguous peptide motifs.

2. The complex of claim 1, wherein the nanoparticle comprises one or more metals selected from the metals in groups 3 to 12 of the periodic table.

3. The complex of claim 2, wherein the one or more metals are selected from the metals in periods 4 to 6 of groups 8 to 11 of the periodic table.

4. The complex of claim 1, wherein the metal nanoparticle is an iron, ruthenium, palladium, or gold nanoparticle.

5. The complex of claim 1, wherein the phosphopeptide is adsorbed to the surface of the metal nanoparticle.

6. The complex of claim 1, wherein the two or more phosphorus-containing groups are bound to amino acids at the equivalent position in each peptide motif.

7. The complex of claim 1, wherein each peptide motif is from 3 to 6 amino acids in length.

8. The complex of claim 7, wherein each peptide motif is 3 amino acids in length.

9. The complex of claim 1, wherein the phosphopeptide is from 6 to 50 amino acids in length.

10. The complex of claim 1, wherein the phosphopeptide further comprises one or more groups that mitigates aggregation of the metal nanoparticle-phosphopeptide complex with metal nanoparticles or other metal nanoparticle-phosphopeptide complexes.

11. The complex of claim 10, wherein the group that mitigates aggregation is a charged peptide.

12. The complex of claim 1, wherein each amino acid of the two or more peptide motifs is independently a natural amino acid; or an unnatural amino acid residue of the formula (II): 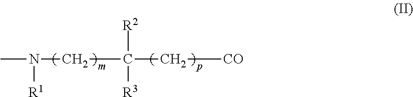
wherein:
R¹ and R³ are each hydrogen;
R² is C₁₋₆alkylheteroaryl; and
m is 0 and p is 0.

13. The complex of claim 12, wherein each phosphorus-containing group is bound to the oxygen atom of a serine, threonine, or tyrosine residue hydroxyl group; or the heteroaryl group of an amino acid residue of the formula (II).

14. The complex of claim 13, wherein each phosphorus-containing group bound to a natural amino acid is —P(O)(OH)₂; and each phosphorus-containing group bound to an unnatural amino acid is C₁₋₆alkylphosphonate.

15. A method for preparing a metal nanoparticle-phosphopeptide complex, the method comprising contacting
a metal compound; and
a phosphopeptide comprising two or more contiguous peptide motifs and two or more phosphorus-containing groups capable of interacting with the surface of the metal nanoparticle,
wherein the amino acids at the equivalent position in each peptide motif have similar structural and/or electronic properties, and
wherein each phosphorus-containing group is bound to an amino acid in the two or more contiguous peptide motifs;
in a liquid reaction medium under conditions that form a metal nanoparticle-phosphopeptide complex.

16. The method of claim 15, wherein the method comprises contacting the metal compound and phosphopeptide with a reducing agent in the liquid reaction medium.

17. The method of claim 16, wherein the metal compound is a metal salt comprising a metal cation.

18. The method of claim 16, wherein the metal compound is a compound of a metal selected from the metals in periods 4 to 6 of groups 8 to 11 of the periodic table.

19. The method of claim 16, wherein the reducing agent is sodium borohydride.

20. The method of claim 16, wherein the liquid reaction medium comprises water.

21. The method of claim 15, wherein the method comprises contacting the metal compound and phosphopeptide under conditions that precipitate the metal nanoparticle-phosphopeptide complex.

22. The method of claim 21, wherein the method comprises contacting the metal compound and phosphopeptide with hydroxide or chalcogen anions.

23. The method of claim 21, wherein the method comprises contacting two or more metal compounds.

24. The method of claim 21, wherein the metal compound is a compound of a metal selected from the metals in groups 3 to 12 of the periodic table.

25. The method of claim 21, wherein the metal compound is an iron (II) or iron (III) salt.
